(19) United States
(12) Patent Application Publication
(10) Pub. No.: US 2026/0115255 A1
(43) Pub. Date: Apr. 30, 2026

(54) INTERLEUKIN 15 VARIANTS

(71) Applicants: Cytune Pharma, Nantes (FR); Sotio Biotech a.s., Prague 9 (CZ)

(72) Inventors: Guy Luc Michel De Martynoff, Mont-St-Guibert (BE); Irena Adkins, Prezletice (CZ); Ulrich Moebius, Gauting-Unterbrunn (DE); David Béchard, Saint-Etienne de Montluc (FR); Eva Nedvedová, Karlovy Vary (CZ); Zuzana Antošová, Rícany (CZ); Šárka Pechoucková, Praha (CZ); Lenka Kyrych Sadilkova, Horomerice (CZ); Roger Renzo Beerli, Adlikon bei Regensdorf (CH); Lukas Bammert, Basel (CH); Lorenz Waldmeier, Nidau (CH); Iva Valentová, Ceské Budejovice (CZ); Simona Hošková, Prague 9-Vinor (CZ)

(21) Appl. No.: 18/573,387

(22) PCT Filed: Jun. 23, 2022
(86) PCT No.: PCT/EP2022/067253

§ 371 Date: Dec. 21, 2023

(30) Foreign Application Priority Data
Jun. 23, 2021 (EP) 21181261.5

(51) Int. Cl. A61K 38/20; A61K 39/00; A61K 39/395; A61P 35/00; C07K 14/54; C07K 16/28

(52) U.S. Cl. CPC A61K 38/2086; A61K 39/39541; A61P 35/00; C07K 14/5443; C07K 16/2818; A61K 2039/505; C07K 2317/732; C07K 2317/92; C07K 2319/00

(57) ABSTRACT

The present invention provides interleukin 15 (IL-15) variants comprising amino acid substitutions for improved homogeneity as well as conjugates and fusion proteins comprising such IL-15 variants. Further, nucleic acids, vectors and host cells for expression of such IL-15 variants as well as pharmaceutical composition comprising such IL-15 variants are provided.

### BACKGROUND OF THE INVENTION

Interleukin 15 (IL-15) is a naturally occurring cytokine that induces the generation of cytotoxic lymphocytes and memory phenotype CD8⁺ T cells, and stimulates proliferation and maintenance of natural killer (NK) cells but—in contrast to interleukin 2—does not mediate activation-induced cell death, does not consistently activate Tregs and causes less capillary leak syndrome (Waldmann et al. 2020). Extensive preclinical and clinical studies demonstrating the effectiveness and limitation of IL-15 and of an increasing number of IL-15 analogs/superagonists especially in the treatment of cancer have been conducted, reviewed by Robinson and Schluns (Robinson and Schluns 2017).

IL-15, like interleukin 2 (IL-2), acts through a heterotrimeric receptor having α, β and γ subunits, whereas they share the common gamma-chain receptor (γ_{c} or γ)—also shared with IL-4, IL-7, IL-9 and IL-21—and the IL-2/IL-15Rβ (also known as IL-2Rβ, CD122). As a third subunit, the heterotrimeric receptors contain a specific subunit for IL-2 or IL-15, i.e., the IL-2Rα (CD25) or the IL-15Rα (CD215). Downstream, IL-2 and IL-15 heterotrimeric receptors share JAK1 (Janus kinase 1), JAK 3, and STAT3/5 (signal transducer and activator of transcription 3 and 5) molecules for intracellular signaling leading to similar functions, but both cytokines also have distinct roles as reviewed in Waldmann (2015, see e.g. table 1) and Conlon (2019).

Accordingly, the activation of different heterotrimeric receptors by binding of IL-2, IL-15 or derivatives thereof potentially leads to a specific modulation of the immune system and potential side effects. Recently, novel compounds comprising IL-15 or IL-15 variants were designed aiming at specifically targeting the activation of NK cells and CD8⁺ T cells. These are compounds targeting the mid-affinity IL-2/IL-15Rβγ, i.e., the receptor consisting of the IL-2/IL-15Rβ and the γ_{c} subunits, which is expressed on NK cells, CD8⁺ T cells, NKT cells and γδ T cells. This is critical for safe and potent immune stimulation mediated by IL-15 trans-presentation, whereas the designed compounds SO-C101 (RLI-15), ALT-803 and hetIL-15 already contain (part of) the IL-15Rα subunit and therefore simulate trans-presentation of the α subunit by antigen presenting cells. SO-C101 binds to the mid-affinity IL-15Rβγ only, as it comprises the covalently attached sushi+ domain of IL-15Rα. In turn, SO-C101 does bind neither to IL-15Rα nor to IL-2Rα. Similarly, ALT-803 and hetIL-15 carry an IL-15Rα sushi domain or the soluble IL-15Rα, respectively, and therefore bind to the mid-affinity IL-15Rβγ receptor. Accordingly, IL-15 and IL-15 analogs/superagonists are promising clinical stage development candidate for the treatment of cancer and infectious diseases.

However, IL-15 and IL-15 superagonists are known to accumulate heterogeneities during expression, purification, storage and delivery, which potentially can adversely affect its pharmaceutical efficacy. Examples of such heterogeneities are different levels of glycosylation, deamidation of asparagines or glutamines or oxidation of histidines, methionines, cysteines, tryptophans or tyrosines, where the amide nitrogen group is exchanged with oxygen thereby changing polar amides into negatively charged carboxylic acids. Such changes induce heterogeneity of the drug product and bare the risk of increased immunogenicity, i.e., generation of anti-drug antibodies that limit the pharmaceutical effect of the drug. Accordingly, there is a continued need to provide variants of IL-15 and IL-15 superagonists with reduced heterogeneity, which however essentially retain their activity and are expressed at a similar level.

### SUMMARY OF THE INVENTION

The inventors have surprisingly identified an IL-15 variant with specific combinations of amino acid substitutions that considerably reduce the deamidation of Asparagine 77 (N77) and the glycosylation of IL-15. The IL-15 variant surprisingly exhibits a similar activity, similar expression levels and, in a fusion protein with interleukin 15 receptor alpha, a longer half-life in vivo compared to mature human IL-15.

Whereas reduced or changed glycosylation patterns may have an impact on activity of the protein in vitro and in vivo, and glycosylation of proteins is generally described to increase half-life in vivo and prevent physical instabilities of proteins (Sola and Griebenow 2009), the inventors surprisingly found out that the combined substitution of G78 and N79 leads to a number of unexpected advantages. Specifically, mutating these two sites results in reduced deamidation, reduced glycosylation and increased homogeneity of the IL-15 variant. At the same time, substituting these amino acid residues has no effect on potency of the IL-15 activity, has no effect on other stability parameters than deamidation and, even has an increased in vivo half-life in a fusion protein with interleukin 15 receptor α, compared to mature IL-15. The latter is particularly advantageous. An increase in in vivo half-life for IL-15 or IL-15/IL-15Rα superagonists is generally seen as beneficial as their half-life is very short and researchers employed various principles to increase the in vivo half-life, as for example forming complexes with the soluble IL-15Rα (WO 2007/001677), coupling an IL-15/IL-15Rα sushi conjugate to an Fc fragment (WO 2008/143794A1), or PEGylate IL-15 (WO 2015/153753a2). Additionally, the inventors found out that such combined substitution led to an increased stability of the IL-15 during the manufacturing process, whereas wildtype IL-15 is degraded under such conditions.

Accordingly, the present invention inter alia provides IL-15 variants and conjugates comprising such IL-15 variants. These variants and conjugates can be used in the treatment of new tumor indications and patient groups.

### Definitions, Abbreviations and Acronyms

“Interleukin-15”, “IL-15” or “IL15” refers to the human cytokine as described by NCBI Reference Sequence NP_000576.1 or UniProt ID P40933 (SEQ ID NO: 1). Its precursor protein has 162 amino acids, having a long 48-aa peptide leader and resulting in a 114-aa mature protein (SEQ ID NO: 2), whereas mature refers to an IL-15 protein where the signal peptide of 48 amino acids of SEQ ID NO: 1 are missing. Its mRNA, complete coding sequence is described by NCBI GenBank Reference U14407.1.

“IL-15 variant” or “variant of IL-15” refers to a protein having a percentage of identity of at least 92%, preferably of at least 96%, more preferably of at least 98%, and most preferably of at least 99% with the amino acid sequence of the mature human IL-15 (114 aa) (SEQ ID NO: 2). Preferably, an IL-15 variant has at least 10% of the activity of IL-15, more preferably at least 25%, even more preferably at least 50%, and most preferably at least 80%. More preferably, the IL-15 variant has at least 0.1% of the activity of human IL-15, preferably 1%, more preferably at least 10%, more preferably at least 25%, even more preferably at least 50%, and most preferably at least 80%. Interleukins are extremely potent molecules working at very low concentrations, even such low activities as 0.1% of human IL-15 may still be sufficiently potent, especially if dosed higher or if an extended half-life compensates for the loss of activity.

The activity of IL-15 can be determined by induction of proliferation of kit225 cells as described by Hori et al. (1987). Preferably, methods such as colorimetry or fluorescence are used to determine proliferation activation due to IL-2 or IL-15 stimulation, as for example described by Soman et al. using CTLL-2 cells (Soman et al. 2009). As an alternative to cell lines such as the kit225 cells, human peripheral blood mononuclear cells (PBMCs) or buffy coats can be used. A preferred bioassay to determine the activity of IL-15 is the IL-2/IL-15 Bioassay Kit using STAT5-RE CTLL-2 cells (Promega Catalog number CS2018B03/B07/B05).

IL-15 muteins can be generated by standard genetic engineering methods and are well known in the art, e.g., from WO 2005/085282, US 2006/0057680, WO 2008/143794, WO 2009/135031, WO 2014/207173, WO 2016/142314, WO 2016/060996, WO 2017/046200, WO 2018/071918, WO 2018/071919, US 2018/0118805. IL-15 variants may further be generated by chemical modification as known in the art, e.g., by PEGylation or other posttranslational modifications (see WO 2017/112528A2, WO 2009/135031A1).

“IL-15Rα” refers to the human IL-15 receptor α or CD215 as described by NCBI Reference Sequence AAI21142.1 or UniProt ID Q13261 (SEQ ID NO: 4). Its precursor protein has 267 amino acids, having a 30-aa peptide leader and resulting in a 231-aa mature protein. Its mRNA is described by NCBI GenBank Reference HQ401283.1. The IL-15Rα sushi domain (or IL-15Rαₛᵤₛₕᵢ, SEQ ID NO: 5) is the domain of IL-15Rα which is essential for binding to IL-15 (Wei et al. 2001). The sushi+ fragment (SEQ ID NO: 6) comprising the sushi domain and part of hinge region, defined as the fourteen amino acids which are located after the sushi domain of this IL-15Rα, in a C-terminal position relative to said sushi domain, i.e., said IL-15Rα hinge region begins at the first amino acid after said (C4) cysteine residue, and ends at the fourteenth amino acid (counting in the standard “from N-terminal to C-terminal” orientation). The sushi+ fragment reconstitutes full binding activity to IL-15 (WO 2007/046006).

“IL-15Rα derivative” refers to a polypeptide comprising an amino acid sequence having a percentage of identity of at least 92%, preferably of at least 96%, more preferably of at least 98%, and even more preferably of at least 99%, and most preferably 100% identical with the amino acid sequence of the sushi domain of human IL-15Rα (SEQ ID NO: 5) and, preferably of the sushi+ domain of human IL-15Rα (SEQ ID NO: 6). Preferably, the IL-15Rα derivative is a N- and C-terminally truncated polypeptide, whereas the signal peptide (amino acids 1-30 of SEQ ID NO: 4) is deleted and the transmembrane domain and the intracytoplasmic part of IL-15Rα is deleted (amino acids 210 to 267 of SEQ ID NO: 4). Accordingly, preferred IL-15Rα derivatives comprise at least the sushi domain (aa 33-93 but do not extend beyond the extracellular part of the mature IL-15Rα being amino acids 31-209 of SEQ ID NO: 4. Specific preferred IL-15Rα derivatives are the sushi domain of IL-15Rα (SEQ ID NO: 5), the sushi+ domain of IL-15Rα (SEQ ID NO: 6) and a soluble form of IL-15Rα (from amino acids 31 to either of amino acids 172, 197, 198, 199, 200, 201, 202, 203, 204 or 205 of SEQ ID NO: 4, see WO 2014/066527, (Giron-Michel et al. 2005)). Within the limits provided by this definition, the IL-15Rα derivative may include natural occurring or introduced mutations. Natural variants and alternative sequences are e.g. described in the UniProtKB entry Q13261 (https://www.uniprot.org/uniprot/Q13261). Further, the artisan can easily identify less conserved amino acids between mammalian IL-15Rα homologs or even primate IL-15Rα homologs to generate derivatives which are still functional. Respective sequences of mammalian IL-15Rα homologs are described in WO 2007/046006, pages 18 and 19. Additionally or alternatively, the artisan can easily make conservative amino acid substitutions.

Preferably, an IL-15Rα derivative has at least 10% of the binding activity of the human sushi domain to human IL-15, e.g., as determined in Wei et al. (2001), more preferably at least 25%, even more preferably at least 50%, and most preferably at least 80%.

“IL-2Rγ” refers to the common cytokine receptor γ or γ_{c} or CD132, shared by IL-4, IL-7, IL-9, IL-15 and IL-21.

“RLI-15” or “RLI” refers to any IL-15/IL-15Rα conjugate being a receptor-linker-interleukin fusion protein of the human IL-15Rα sushi+ fragment with the human IL-15. Suitable linkers are described in WO 2007/046006 and WO 2012/175222.

“RLI2” or “SO-C101” are specific versions of RLI-15 and refer to an IL-15/IL-15Rα conjugate being a receptor-linker-interleukin fusion protein of the human IL-15Rα sushi+ fragment with the human IL-15 (SEQ ID NO: 8) using the linker with the SEQ ID NO: 7.

A conjugate, as used herein, relates to either a non-covalent or a covalent complex of an interleukin 15 (IL-15) or a derivative thereof and the sushi domain of an interleukin 15-receptor alpha (IL-15Rα) or a derivative thereof. The non-covalent complex may be formed either by co-expression of the two polypeptides or by separate expression, (partial) purification and subsequent combination to form such complex due to the affinity of such polypeptides. Preferably, the conjugate is a fusion polypeptide or protein, where at least two polypeptides are genetically fused and recombinantly expressed to result in a single polypeptide chain to form the intact complex.

According to this invention, a fusion polypeptide or protein includes conjugates with at least one fusion polypeptide non-covalently or preferably covalently linked to another polypeptide chain, e.g. an immunocytokine comprising an antibody (with two heavy chains and two light chains covalently linked through disulfide bonds) fused with an IL-15 or variant thereof, or an IL-15/sushi domain fusion protein, or an Fc domain of an antibody having two CH2/CH3 comprising polypeptide chains each fused to a sushi domain each complexed with an IL-15 variant, or one CH2/CH3 comprising polypeptide being fused to a sushi domain, the other being fused to an IL-15.

An immunocytokine, as used herein, relates to polypeptide comprising an antibody or a functional variant thereof, genetically fused to a conjugate according to the invention.

“ALT-803” refers to an IL-15/IL-15Rα conjugate of Altor BioScience Corp., which is a conjugate containing 2 molecules of an optimized amino acid-substituted (N72D) human IL-15 “superagonist”, 2 molecules of the human IL-15a receptor “sushi” domain fused to a dimeric human IgG1 Fc that confers stability and prolongs the half-life of the IL-15_{N72D}:IL-15Rαₛᵤₛₕᵢ-Fc conjugate (see for example US 2017/0088597).

“P-22339” refers to an IL-15/IL-15Rα conjugate of Hengrui Medicine, which is a fusion protein containing 2 fusions of an IL-15 with the sushi domain of IL-15Rα through an engineered disulfide bond fused to the N-termini of an Fc fragment.

“XmAb24306” refers to an IL-15/IL-15Rα conjugate of Xencor, which is a fusion protein where a sushi domain of IL-15Rα and an IL-15 are fused to the N-termini of an Fc fragment.

“CUG105” refers to an IL-15/IL-15Rα conjugate of Cugene, which is a fusion protein where a sushi domain of IL-15Rα and an IL-15 are fused to the N-termini of an Fc fragment.

“Heterodimeric IL-15:IL-Rα”, “hetIL-15” or “NIZ985” refer to an IL-15/IL-15Rα conjugate of Novartis which resembles the IL-15, which circulates as a stable molecular conjugate with the soluble IL-15Rα, which is a recombinantly co-expressed, non-covalent conjugate of human IL-15 and the soluble human IL-15Rα (sIL-15Rα), i.e. 170 amino acids of IL-15Rα without the signal peptide and the transmembrane and cytoplasmic domain (Thaysen-Andersen et al. 2016, see e.g. table 1).

“IL-2/IL-15Rβγ agonists” refers to molecules or conjugates which primarily target the mid-affinity IL-2/IL-15Rβγ receptor without binding to the IL-2Rα and/or IL-15Rα receptor, thereby lacking a stimulation of T_{regs}. Examples are IL-15 bound to at least the sushi domain of the IL-15Rα having the advantage of not being dependent on trans-presentation or cell-cell interaction, and of a longer in vivo half-life due to the increased size of the molecule, which have been shown to be significantly more potent that native IL-15 in vitro and in vivo (Robinson and Schluns 2017). Besides IL-15/IL-15Rα based conjugates, this can be achieved by mutated or chemically modified IL-2, which have a markedly reduced or timely delayed binding to the IL-2a receptor without affecting the binding to the IL-2/15Rβ and γ_{c} receptor.

“NKTR-255” refers to an IL-2/IL-15Rβγ agonist based on a PEG-conjugated human IL-15 that retains binding affinity to the IL-15Rα and exhibits reduced clearance to provide a sustained pharmacodynamic response (WO 2018/213341A1).

“THOR-924, -908, -918” refer to IL-2/IL-15Rβγ agonists based on PEG-conjugated IL-15 with reduced binding to the IL-15Rα with a unnatural amino acid used for site-specific PEGylation (WO 2019/165453A1).

“AM0015” refers to a PEG-conjugated IL-15 mutein (WO 2017/112528).

“Percentage of identity” between two amino acids sequences means the percentage of identical amino acids, between the two sequences to be compared, obtained with the best alignment of said sequences, this percentage being purely statistical and the differences between these two sequences being randomly spread over the amino acids sequences. As used herein, “best alignment” or “optimal alignment”, means the alignment for which the determined percentage of identity (see below) is the highest. Sequences comparison between two amino acids sequences are usually realized by comparing these sequences that have been previously aligned according to the best alignment; this comparison is realized on segments of comparison to identify and compare the local regions of similarity. The best sequences alignment to perform comparison can be realized, beside by a manual way, by using the global homology algorithm developed by Smith and Waterman (1981), by using the local homology algorithm developed by Needleman and Wunsch (1970), by using the method of similarities developed by Pearson and Lipman (1988), by using computer software using such algorithms (GAP, BESTFIT, BLAST P, BLAST N, FASTA, TFASTA in the Wisconsin Genetics software Package, Genetics Computer Group, 575 Science Dr., Madison, WI USA), by using the MUSCLE multiple alignment algorithms (Edgar 2004), or by using CLUSTAL (Goujon et al. 2010). To get the best local alignment, one can preferably use the BLAST software with the BLOSUM 62 matrix. The identity percentage between two sequences of amino acids is determined by comparing these two sequences optimally aligned, the amino acids sequences being able to encompass additions or deletions in respect to the reference sequence to get the optimal alignment between these two sequences. The percentage of identity is calculated by determining the number of identical positions between these two sequences and dividing this number by the total number of compared positions, and by multiplying the result obtained by 100 to get the percentage of identity between these two sequences.

“Conservative amino acid substitutions” refers to a substation of an amino acid, where an aliphatic amino acid (i.e. Glycine, Alanine, Valine, Leucine, Isoleucine) is substituted by another aliphatic amino acid, a hydroxyl or sulfur/selenium-containing amino acid (i.e. Seine, Cysteine, Selenocysteine, Threonine, Methionine) is substituted by another hydroxyl or sulfur/selenium-containing amino acid, an aromatic amino acid (i.e. Phenylalanine, Tyrosine, Tryptophan) is substituted by another aromatic amino acid, a basic amino acid (i.e. Histidine, Lysine, Arginine) is substituted by another basic amino acid, or an acidic amino acid or its amide (Aspartate, Glutamate, Asparagine, Glutamine) is replaced by another acidic amino acid or its amide.

“Antibody” also known as an immunoglobulin (Ig) is a large, Y-shaped protein composed in humans and most mammals of two heavy chains (HC) and two light chains (LC) connected by disulfide bonds. Light chains consist of one variable domain V_{L} and one constant domain C_{L}, while heavy chains contain one variable domain V_{H} and three constant domains C_{H}1, C_{H}2, C_{H}3. Structurally an antibody is also partitioned into two antigen-binding fragments (Fab), containing one V_{L}, V_{H}, C_{L}, and C_{H}1 domain each, as well as the Fc fragment or domain containing the two C_{H}2 and C_{H}3 of the two heavy chains.

An “antibody variant” or “antibody functional variant”, as used herein, relates to antibodies with modifications for e.g., modulating their effector functions, modulating the antibody stability and in vivo half-life and/or inducing heterodimerization of the antibody Fc domains. Such variants may be achieved by mutations and/or posttranslational modifications. Antibody variants also include antibody heavy chains with truncation of the N-terminal lysine on one or preferably both heavy chains. Other included variations are N- or C-terminal tags of the heavy and/or light chains for chemical or enzymatic coupling to other moieties such as dyes, radionuclides, toxins or other binding moieties. Further, antibody variants may comprise chemical modifications, modifications of their glycosylation or substitutions with artificial amino acids for chemical linkage to other moieties. Antibody variant, as used herein, also relates to immunoglobulin gamma (IgG)-based bispecific antibodies that potentially recognize two or more different epitopes. Various formats of bispecific antibodies are known in the art, e.g. reviewed by Godar et al. (2018) and Spiess et al. (2015). Bispecific formats according to this invention include an Fc domain. With respect to the immunocytokines of the inventions, two RLI conjugates may, if not otherwise linked to a moiety, be either fused to the C-terminus of both light chains or to the C-terminus of both heavy chains; alternatively, one RLI conjugate may be fused to the C-terminus of one heavy chain for heterodimeric bispecific formats, or to the heavy chain or one light chain of heterodimeric bispecific formats with different light chains. Antibody functional variants are capable of binding to the same epitope or target as their corresponding non-modified antibody.

“In vivo half-life”, T½ or terminal half-life refers to the half-life of elimination or half-life of the terminal phase, i.e. following administration the in vivo half-life is the time required for plasma/blood concentration to decrease by 50% after pseudo-equilibrium of distribution has been reached (Toutain and Bousquet-Melou 2004). The determination of the drug, here the IL-2/IL-15βγ agonist being a polypeptide, in the blood/plasma is typically done through a polypeptide-specific ELISA.

“Immune check point inhibitor”, or in short “check point inhibitors”, refers to a type of drug that blocks certain proteins (immune checkpoint proteins) made by some types of immune system cells, such as T cells, and some cancer cells. These proteins are important for maintaining peripheral tolerance and preventing excessive immune reactions. In malignant diseases these proteins can be employed by tumor cells to prevent T cells from killing cancer cells. When these proteins are blocked by check point inhibitors, the “brakes” on the immune system are released and T cells are able to kill cancer cells again. Checkpoint inhibitors are accordingly antagonists of immune inhibitory checkpoint molecules or antagonists of agonistic ligands of inhibitory checkpoint molecules. Examples of checkpoint proteins found on T cells or cancer cells include PD-1/PD-L1 and CTLA-4/B7-1/B7-2 (definition of the National Cancer Institute at the National Institute of Health, see https://www.cancer.gov/publications/dictionaries/cancer-terms/def/immune-checkpoint-inhibitor), as for example reviewed by Darvin et al. (2018). Examples of check point inhibitors are anti-PD-L1 antibodies, anti-PD-1 antibodies, anti-CTLA-4 antibodies, but also antibodies against LAG-3 or TIM-3, or blocker of BTLA currently tested in the clinic (De Sousa Linhares et al. 2018). Further promising check point inhibitors are anti-TIGIT antibodies (Solomon and Garrido-Laguna 2018).

“anti-PD-L1 antibody” refers to an antibody, or an antibody fragment thereof, binding to PD-L1. Examples are avelumab, atezolizumab, durvalumab, KN035, MGD013 (bispecific for PD-1 and LAG-3).

“anti-PD-1 antibody” refers to an antibody, or an antibody fragment thereof, binding to PD-1. Examples are pembrolizumab, nivolumab, cemiplimab (REGN2810), BMS-936558, SHR1210, 1B1308, PDR001, BGB-A317, BCD-100 and JS001.

“anti-PD-L2 antibody” refers to an antibody, or an antibody fragment thereof, binding to anti-PD-L2. An example is sHIgM12.

“anti-CTLA4 antibody” refers to an antibody, or an antibody fragment thereof, binding to CTLA-4. Examples are ipilimumab and tremelimumab (ticilimumab).

“anti-LAG-3” antibody refers to an antibody, or an antibody fragment thereof, binding to LAG-3. Examples of anti-LAG-3 antibodies are relatlimab (BMS 986016), Sym022, REGN3767, TSR-033, GSK2831781, MGD013 (bispecific for PD-1 and LAG-3) and LAG525 (IMP701).

“anti-TIM-3 antibody” refers to an antibody, or an antibody fragment thereof, binding to TIM-3. Examples are TSR-022 and Sym023.

“anti-TIGIT antibody” refers to an antibody, or an antibody fragment thereof, binding to TIGIT. Examples are tiragolumab (MTIG7192A, RG6058) and etigilimab (WO 2018/102536).

“Therapeutic antibody” or “tumor targeting antibody” refers to an antibody, or an antibody fragment thereof, that has a direct therapeutic effect on tumor cells through binding of the antibody to the target expressed on the surface of the treated tumor cell. Such therapeutic activity may be due to receptor binding leading to modified signaling in the cell, direct cell death induction, antibody-dependent cellular cytotoxicity (ADCC), complement-dependent cytotoxicity (CDC) or other antibody-mediated killing of tumor cells.

“anti-CD38 antibody” refers to an antibody, or an antibody fragment thereof, binding to CD38, also known as cyclic ADP ribose hydrolase. Examples of anti-CD38 antibodies are daratumumab, isatuximab (SAR650984), MOR-202 (MOR03087), TAK-573 or TAK-079 (Abramson 2018) or GEN1029 (HexaBody®-DR5/DR5).

When it is stated “administered in combination” this typically does not mean that the two agents are co-formulated and co-administered, but rather one agent has a label that specifies its use in combination with the other. So, for example the IL-2/IL-15Rβγ agonist is for use in treating or managing cancer, wherein the use comprises simultaneously, separately, or sequentially administering the IL-2/IL-15Rβγ agonist and a further therapeutic agent, or vice versa. But nothing in this application should exclude those two combined agents being provided as a bundle or kit, or even are co-formulated and administered together where dosing schedules match. So, “administered in combination” includes (i) that the drugs are administered together in a joint infusion, in a joint injection or alike, (ii) that the drugs are administered separately but in parallel according to the given way of administration of each drug, and (iii) that the drugs are administered separately and sequentially. Parallel administration in this context preferably means that both treatments are initiated together, e.g. the first administration of each drug within the treatment regimen are administered on the same day. Given potential different treatment schedules it is clear that in the course of following days/weeks/months administrations may not always occur on the same day. In general, parallel administration aims for both drugs being present in the body at the same time at the beginning of each treatment cycle.

Sequential administration in this context preferably means that both treatments are started sequentially, e.g., the first administration of the first drug occurs at least one day, preferably a few days or one week, earlier than the first administration of the second drug in order to allow a pharmacodynamic response of the body to the first drug before the second drug becomes active. Treatment schedules may then be overlapping or intermittent, or directly following each other.

The term “resistant to checkpoint inhibitor treatment” refers to a patient that never showed a treatment response when receiving a checkpoint inhibitor.

The term “refractory to checkpoint inhibitor treatment” refers to a patient that initially showed a treatment response to checkpoint inhibitor treatment, but the treatment response was not maintained over time.

The term “about”, when used together with a value, means the value plus/minus 10%, preferably 5% and especially 1% of its value.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is also to be understood to disclose a group, which preferably consists only of these embodiments.

Where an indefinite or definite article is used when referring to a singular noun, e.g. “a”, “an” or “the”, this includes a plural of that noun unless something else is specifically stated.

The term “at least one” such as in “at least one chemotherapeutic agent” may thus mean that one or more chemotherapeutic agents are meant. The term “combinations thereof” in the same context refers to a combination comprising more than one chemotherapeutic agent.

Technical terms are used by their common sense. If a specific meaning is conveyed to certain terms, definitions of terms will be given in the following in the context of which the terms are used.

“qxw”, from Latin quaque/each, every for every x week, e.g., q2w for every second week.

“s.c.” or “SC” for subcutaneously.

“i.v.” or “IV” for intravenously.

“i.p.” or “IP” for intraperitoneally.

Cₘₐₓ for maximal concentration

AUC for area under the curve.

TABLE 1
list of molecules

| Molecule | Molecule characteristics |
|---|---|
| -RLI x1 | RLI2 conjugated to the Cₜ of the “knob” antibody heavy chain |
| -RLI x2 | RLI2 conjugated to the Cₜ of both antibody heavy chains |
| IL-15_{AQ} | IL-15 mutations: G78A, N79Q |
| IL-15_{AQA} | IL-15 mutations: N65A, G78A, N79Q |
| RLI2_{AQ} | RLI-15 mutations: G175A, N176Q |
| RLI2_{AQA} | RLI-15 mutations: N162A, G175A, N176Q |
| SOT201 | RLI-15 mutations: N162A (N65A), G175A (G78A), N176Q (N79Q) |
| (PEM L-RLI NA x1) | Pembrolizumab (“PEM”) with Fc modification: |
| | L235E, S228P, |
| | T366W (C_{H}3A chain “Knob”), |
| | T366S, L368A, Y407V (C_{H}3B chain “hole”) |
| PEM L-RLI DANA x1 | RLI-15 mutations: D158A (D61A), N162A, G175A, N176Q |
| (SOT201-DANA) | Pembrolizumab (“PEM”) with Fc modification: |
| | L235E, S228P, |
| | T366W (C_{H}3A chain “Knob”), |
| | T366S, L368A, Y407V (C_{H}3B chain “hole”) |
| PEM LY-RLI DANA x1 | RLI-15 mutations: D158A, N162A , G175A, N176Q |
| | Pembrolizumab (“PEM”) with Fc modification: |
| | L235E, M252Y/S254T/T256E, S228P, |
| | T366W (C_{H}3A chain “Knob”), |
| | T366S, L368A, Y407V (C_{H}3B chain “hole”) |
| PEM LY-RLI | RLI-15 mutations: D158A, N162A (N65A), Q198D (Q101D), G175A, |
| DANAQD x1 | N176Q |
| | Pembrolizumab (“PEM”) with Fc modification: |
| | L235E, M252Y/S254T/T256E, S228P, |
| | T366W (C_{H}3A chain “Knob”), |
| | T366S, L368A, Y407V (C_{H}3B chain “hole”) |
| PEM LE-RLI x1 | Pembrolizumab (“PEM”) with Fc modification: |
| | L235E, S228P, |
| | T366W (C_{H}3A chain “Knob”), |
| | T366S, L368A, Y407V (C_{H}3B chain “hole”) |
| PEM LE-RLI x2 | Pembrolizumab (“PEM”) with Fc modification: |
| | L235E, S228P, |
| | Pembrolizumab with RLI2 conjugated to the Cₜ of both antibody heavy |
| | chains |
| PEM LE-RLI-AD x1 | RLI-15 mutations: K10A, Q101D |
| | Pembrolizumab (“PEM”) with Fc modification: |
| | L235E, S228P, |
| | T366W (C_{H}3A chain “Knob”), |
| | T366S, L368A, Y407V (C_{H}3B chain “hole”) |
| PEM LE-RLI-AD x2 | RLI-15 mutations: K10A, Q101D |
| | Pembrolizumab (“PEM”) with Fc modification: |
| | L235E, S228P |
| PEM LE-RLI-DA x1 | RLI-15 mutations: D61A |
| | Pembrolizumab (“PEM”) with Fc modification: |
| | L235E, S228P, |
| | T366W (C_{H}3A chain “Knob”), |
| | T366S, L368A, Y407V (C_{H}3B chain “hole”) |
| PEM LE-RLI-DA x2 | RLI-15 mutations: D61A |
| | Pembrolizumab (“PEM”) with Fc modification: |
| | L235E, S228P |
| PEM LE-RLI-NA x1 | RLI-15 mutations: N65A |
| | Pembrolizumab (“PEM”) with Fc modification: |
| | L235E, S228P |
| | T366W (C_{H}3A chain “Knob”), |
| | T366S, L368A, Y407V (C_{H}3B chain “hole”) |
| PEM LE-RLI-NA x2 | RLI-15 mutations: N65A |
| | Pembrolizumab (“PEM”) with Fc modification: |
| | L235E, S228P |
| PEM LE-RLI-ND x1 | RLI-15 mutations: N65D |
| | Pembrolizumab (“PEM”) with Fc modification: |
| | L235E, S228P |
| | T366W (C_{H}3A chain “Knob”), |
| | T366S, L368A, Y407V (C_{H}3B chain “hole”) |
| PEM LE-RLI-ND x2 | RLI-15 mutations: N65D |
| | Pembrolizumab (“PEM”) with Fc modification: |
| | L235E, S228P |
| PEM LE-RLI-NQD x1 | RLI-15 mutations: D30N, E64Q, N65D |
| | Pembrolizumab (“PEM”) with Fc modification: |
| | L235E, S228P |
| | T366W (C_{H}3A chain “Knob”), |
| | T366S, L368A, Y407V (C_{H}3B chain “hole”) |
| PEM LE-RLI-NQD x2 | RLI-15 mutations: D30N, E64Q, N65D |
| | Pembrolizumab (“PEM”) with Fc modification: |
| | L235E, S228P |
| PEM YTE-RLI x1 | Pembrolizumab (“PEM”) with Fc modification: |
| | M252Y, S254T, T256E, S228P, |
| | T366W (C_{H}3A chain “Knob”), |
| | T366S, L368A, Y407V (C_{H}3B chain “hole”) |
| PEM LE/YTE-RLI x1 | Pembrolizumab (“PEM”) with Fc modification: |
| | L235E, M252Y, S254T, T256E, S228P, |
| | T366W (C_{H}3A chain “Knob”), |
| | T366S, L368A, Y407V (C_{H}3B chain “hole”) |
| PEM YTE-RLI NA x1 | RLI-15 mutations: N65A |
| | Pembrolizumab (“PEM”) with Fc modification: |
| | M252Y, S254T, T256E, S228P, |
| | T366W (C_{H}3A chain “Knob”), |
| | T366S, L368A, Y407V (C_{H}3B chain “hole”) |
| PEM LE/YTE-RLI NA | RLI-15 mutations: N65A |
| x1 | Pembrolizumab (“PEM”) with Fc modification: |
| | L235E, M252Y, S254T, T256E, S228P, |
| | T366W (C_{H}3A chain “Knob”), |
| | T366S, L368A, Y407V (C_{H}3B chain “hole”) |
| PEM LE/YTE-RLI | RLI-15 mutations: Q101D, Q108A |
| QDQA x1 | Pembrolizumab (“PEM”) with Fc modification: |
| | L235E, M252Y, S254T, T256E, S228P, |
| | T366W (C_{H}3A chain “Knob”), |
| | T366S, L368A, Y407V (C_{H}3B chain “hole”) |
| PEM LE/YTE-RLI | RLI-15 mutations: D61A, N65A |
| DANA x1 | Pembrolizumab (“PEM”) with Fc modification: |
| | L235E, M252Y, S254T, T256E, S228P, |
| | T366W (C_{H}3A chain “Knob”), |
| | T366S, L368A, Y407V (C_{H}3B chain “hole”) |
| PEM LE/YTE-RLI | RLI-15 mutations: D61A, N65A, Q101D |
| DANAQD x1 | Pembrolizumab (“PEM”) with Fc modification: |
| | L235E, M252Y, S254T, T256E, S228P, |
| | T366W (C_{H}3A chain “Knob”), |
| | T366S, L368A, Y407V (C_{H}3B chain “hole”) |
| RTX-RLI2 x2 | Rituximab with RLI2 conjugated to the Cₜ of both antibody heavy |
| | chains |
| RTX-RLI2 x1 | Rituximab with RLI2 conjugated to the Cₜ of the “knob” antibody heavy |
| | chain |
| | Rituximab with Fc modifications: |
| | T366W (C_{H}3A chain “Knob”), |
| | T366S, L368A, Y407V (C_{H}3B chain “hole”) |
| RTX-RLI2 AQ x2 | RLI-15 mutations: G175A, N176Q |
| RTX-RLI2 AQ x1 | RLI-15 mutations: G175A, N176Q |
| | Rituximab with Fc modifications: |
| | T366W (C_{H}3A chain “Knob”), |
| | T366S, L368A, Y407V (C_{H}3B chain “hole”) |
| RTX-L40-RLI2 x2 | Rituximab with RLI2 conjugated to the Cₜ of both antibody heavy |
| | chains |
| | L40: linker of 40 amino acids between the Cₜ of the antibody and RLI2 |
| RTX-L40-RLI2 AQ x2 | RLI-15 mutations: G175A, N176Q |
| | Rituximab with RLI2 conjugated to the Cₜ of both antibody heavy |
| | chains |
| | L40: linker of 40 amino acids between the Cₜ of the antibody and RLI2 |
| hCl1a LALA | hCl1a anti-Claudin18.2 antibody with Fc modifications: |
| | L234A, L235A |
| hCl1a LALAPG | hCl1a anti-Claudin18.2 antibody with Fc modifications: |
| | L234A, L235A, P329G |
| hCl1a-RLI DANA | hCl1a with RLI2 conjugated to the Cₜ of the “knob” antibody heavy |
| (SOT202-DANA) | chain |
| | RLI-15 mutations: D61A, N65A |
| | hCl1a with Fc modification: |
| | T366W (C_{H}3A chain “Knob”), |
| | T366S, L368A, Y407V (C_{H}3B chain “hole”) |
| hCl1a DLE-RLI DANA | hCl1a with RLI2 conjugated to the Cₜ of the “knob” antibody heavy |
| (SOT202-DLE-DANA) | chain |
| | RLI-15 mutations: D61A, N65A |
| | hCl1a with Fc modification: |
| | S239D, A330L, I332E |
| | T366W (C_{H}3A chain “Knob”), |
| | T366S, L368A, Y407V (C_{H}3B chain “hole”) |
| hCl1a DE-RLI DANA | hCl1a with RLI2 conjugated to the Cₜ of the “knob” antibody heavy |
| (SOT202-DE-DANA) | chain |
| | RLI-15 mutations: D61A, N65A |
| | hCl1a with Fc modification: |
| | S239D, I332E |
| | T366W (C_{H}3A chain “Knob”), |
| | T366S, L368A, Y407V (C_{H}3B chain “hole”) |
| hCl1a AAA-RLI DANA | hCl1a with RLI2 conjugated to the Cₜ of the “knob” antibody heavy |
| (SOT202-AAA-DANA) | chain |
| | RLI-15 mutations: D61A, N65A |
| | hCl1a with Fc modification: |
| | S298A, E333A, K334A |
| | T366W (C_{H}3A chain “Knob”), |
| | T366S, L368A, Y407V (C_{H}3B chain “hole”) |
| hCl1a TL-RLI DANA | hCl1a with RLI2 conjugated to the Cₜ of the “knob” antibody heavy |
| | chain |
| | RLI-15 mutations: D61A, N65A |
| | hCl1a with Fc modification: |
| | K392T, P396L |
| | T366W (C_{H}3A chain “Knob”), |
| | T366S, L368A, Y407V (C_{H}3B chain “hole”) |
| hCl1a IE-RLI DANA | hCl1a with RLI2 conjugated to the Cₜ of the “knob” antibody heavy |
| | chain |
| | RLI-15 mutations: D61A, N65A |
| | hCl1a with Fc modification: |
| | V264I, I332E |
| | T366W (C_{H}3A chain “Knob”), |
| | T366S, L368A, Y407V (C_{H}3B chain “hole”) |
| hCl1a-RLI DANA afuc | Afucosylated hCl1a with RLI2 conjugated to the Cₜ of the “knob” |
| (SOT202-afuc-DANA) | antibody heavy chain |
| | RLI-15 mutations: D61A, N65A |
| | hCl1a with Fc modification: |
| | T366W (C_{H}3A chain “Knob”), |
| | T366S, L368A, Y407V (C_{H}3B chain “hole”) |
| hCl1a DLE-RLI DANA | Afucosylated hCl1a with RLI2 conjugated to the Cₜ of the “knob” |
| afuc | antibody heavy chain |
| | RLI-15 mutations: D61A, N65A |
| | hCl1a with Fc modification: |
| | S239D, A330L, I332E |
| | T366W (C_{H}3A chain “Knob”), |
| | T366S, L368A, Y407V (C_{H}3B chain “hole”) |
| hCl1a DE-RLI DANA | Afucosylated hCl1a with RLI2 conjugated to the Cₜ of the “knob” |
| afuc | antibody heavy chain |
| | RLI-15 mutations: D61A, N65A |
| | hCl1a with Fc modification: |
| | S239D, I332E |
| | T366W (C_{H}3A chain “Knob”), |
| | T366S, L368A, Y407V (C_{H}3B chain “hole”) |
| hCl1a AAA-RLI DANA | Afucosylated hCl1a with RLI2 conjugated to the Cₜ of the “knob” |
| afuc | antibody heavy chain |
| | RLI-15 mutations: D61A, N65A |
| | hCl1a with Fc modification: |
| | S298A, E333A, K334A |
| | T366W (C_{H}3A chain “Knob”), |
| | T366S, L368A, Y407V (C_{H}3B chain “hole”) |
| SOT202 (hCl1a-RLI | hCl1a with RLI2 conjugated to the Cₜ of the “knob” antibody heavy |
| NA) | chain |
| | RLI-15 mutations: N65A |
| | hCl1a with Fc modification: |
| | T366W (C_{H}3A chain “Knob”), |
| | T366S, L368A, Y407V (C_{H}3B chain “hole”) |
| hCl1a-RLI NA afuc | Afucosylated hCl1a with RLI2 conjugated to the Cₜ of the “knob” |
| | antibody heavy chain |
| | RLI-15 mutations: N65A |
| | hCl1a with Fc modification: |
| | T366W (C_{H}3A chain “Knob”), |
| | T366S, L368A, Y407V (C_{H}3B chain “hole”) |
| hCl1a LALAPG-RLI | hCl1a with RLI2 conjugated to the Cₜ of the “knob” antibody heavy |
| NA | chain |
| (SOT202-LALAPG) | RLI-15 mutations: N65A |
| | hCl1a with Fc modification: |
| | L234A, L235A, P329G |
| | T366W (C_{H}3A chain “Knob”), |
| | T366S, L368A, Y407V (C_{H}3B chain “hole”) |
| hCl1a LALAPG-RLI | hCl1a with RLI2 conjugated to the Cₜ of the “knob” antibody heavy |
| DANA | chain |
| (SOT202-LALAPG- | RLI-15 mutations: D61A, N65A |
| DANA) | hCl1a with Fc modification: |
| | L234A, L235A, P329G |
| | T366W (C_{H}3A chain “Knob”), |
| | T366S, L368A, Y407V (C_{H}3B chain “hole”) |

### DESCRIPTION OF THE INVENTION

In a first aspect, the present invention relates to an interleukin-15 (IL-15) variant comprising amino acid substitutions at position G78 and at position N79 of the mature human IL-15 (SEQ ID NO: 2). Preferably, the substituting amino acid is a naturally occurring amino acid.

The inventors successfully produced an IL-15 variant with inter alia a high homogeneity and reduced glycosylation by substituting sites G 87 and N79, whereas the potency and stability of the IL-15 variant was not affected. This was surprising as glycosylation is the primary cause of microheterogeneity in proteins (glycoforms) and glycoforms reflect complexity at both molecular and cellular levels. There are many potential functions of glycosylation such as protein folding, trafficking, packing, stabilization, protease protection, quaternary structure or organization of water structure. For example, changes in sugar motifs may both reflect and result in physiological changes, e.g., in cancer and rheumatoid arthritis. Therefore, especially for applications as medicinal products, the skilled person is hesitant to modify the glycosylation of a therapeutic protein.

In one embodiment the IL-15 variant comprises the amino acid substitutions G78A, G78V, G78L or G78I, and N79Q, N79H or N79M, preferably G78A and N79Q. The G78A/N79Q double substitution resulted in a superior IL-15 variant upon testing it in the context of the RLI2 fusion protein (here respective numbering would be G175A/N176Q) with respect to homogeneity, stability and in vivo half-life.

Preferably, the IL-15 variant has been expressed in a mammalian cell line, preferably the mammalian cell line is selected from CHO cells, HEK293 cells, COS cells, PER.C6 cells, SP20 cells, NSO cells or any cells derived therefrom, more preferably CHO cells. Whereas various eukaryotic or preferably mammalian expression systems can be employed, expression in CHO cells is the best established expression system and results in good yields.

The amino acid substitutions in the IL-15 variant preferably reduce deamidation at N77 and glycosylation at N79 of the IL-15 variant as compared to the mature human IL-15 without such substitutions. More preferably, there is less than 30% of glycosylated IL-15 variant, especially less than 25% of glycosylated IL-15 variant as determined in the RLI2 fusion. For comparison, RLI2 (without the AQ substitution) has up to 40% glycosylation. In one embodiment, less than 30% of the IL-15 variant is glycosylated. In a further embodiment, less than 25% of the IL-15 variant is glycosylated. Preferably, N71 is more glycosylated compared to IL-15 without such substitution (human mature IL-15). Accordingly, whereas the overall glycosylation of the RLI2 AQ is reduced compared to RLI2, it appears that the glycosylation on the minor glycosylation site N71 (IL-15 numbering)/N168 (RLI numbering) is increased to 20%, likely due to the proximity of the two glycosylation sites N168 and N176, leading to interference with a domination/preferential glycosylation of N176. This interference is lifted with the N176Q substitution leading to increased glycosylation at N168.

In a preferred embodiment, the amino acid substitutions of the IL-15 variant do not substantially reduce the IL-15 activity of the IL-15 variant on the proliferation induction of Kit225 cells, 32Db cells, human PBMC or in the Promega IL-15-bioassay. Substantially in this context means that the activity is not reduced by more than 20%, preferably not more than 10% as compared to the IL-15 without such substitutions. Kit225 cells (Hori et al. 1987) are commonly used to determine induction of proliferation by IL-15 and IL-15 superagonists. Preferably, methods such as colorimetry or fluorescence are used to determine proliferation activation due to IL-2 or IL-15 stimulation, as for example described by Soman et al. using CTLL-2 cells (Soman et al. 2009). As an alternative to cell lines such as the kit225 cells, 32Db cells (ThermoFisher), human peripheral blood mononuclear cells (PBMCs) or buffy coats can be used. A preferred bioassay to determine the activity of IL-15 is the IL-2/IL-15 Bioassay Kit using STAT5-RE CTLL-2 cells (Promega Catalog number CS2018B03/B07/B05).

In another embodiment, the IL-15 variant does not have a substitution at position N71 and/or at position N77. The inventors found out that substituting minor glycosylation sites lead to low expression and glycosylation at other sites. Additionally, with every further mutation/substitution being introduced the risk of immunogenicity is increased, which should be avoided.

In a preferred embodiment, the IL-15 variant comprises at least one further substitution that reduces the binding to the IL-2/IL-15Rβ and/or to the γ_{c} receptor and/or the IL-15Rα. Relating to binding to the IL-2/IL-15Rβ and/or to the γ_{c} receptor: Based on the very high affinity of IL-15 to its receptors, administered IL-15, and similarly an IL-15/IL-15Rα conjugate, show a very short half-life mainly due to target-mediated drug deposition (TMDD), where the drug is bound and thereby consumed and cleared by its target immune cells (Hangasky et al. 2020). Accordingly, single i.v. infusion leads to high Cₘₐₓ and an immediate steep decline with a very short half-life leading to a rather small AUC and therefore a suboptimal pharmacokinetic (PK) profile. However, strong immune cell expansion requires repeated and/or longer IL-15 exposure above a certain threshold, i.e., a higher AUC. There are multiple ways employed to achieve a more preferred PK profile including (i) continuous i.v. infusion, which is however inconvenient, (ii) increasing the size of the molecule, e.g. by PEGylation (e.g. NKTR-255, THOR-924, AM0015), complexing it with part of the IL-15Rα (RLI-15, hetIL-15, ALT-803, P-22339, XmAb24306 or CUG105), or complexing/fusing it with an Fc part of an antibody (ALT-803, P-22339, XmAb24306 or CUG105), (iii) s.c. administration leading to some delayed resorption from the subcutaneous depot, and/or (iv) by decreasing the binding affinity of IL-15 to its receptors and thereby decreasing the TMDD. Such decreased binding of IL-15 to its receptors goes along with a decreased potency for activating its target immune cells in vitro (where TMDD does not play a significant role, e.g. as measured on kit225 cells), but is compensated in vivo by its better PK profile due to the extended in vivo half-life (US 2018/0118805A1) (Bernett et al. 2018).

Suitable amino acid substitutions that reduce binding to the IL-2/IL-15Rβ or the γ_{c} receptor are preferably located at the IL-2Rβ or γ_{c} interface. A number of sites for the further substitution reducing binding to the IL-2/IL-15Rβ and/or to the γc receptor have been described in the prior art. The amino acid substitutions may be one or more sites selected from the list consisting of N1, N4, S7, D8, K10, K11, D30, D61, E64, N65, L69, N72, E92, Q101, Q108, I111, preferably selected from positions D61, N65 and Q101 (see WO 2005/085282, WO 2006/020849A2, WO 2008/143794A1, WO 2014/207173A1, US 2018/0118805A1) (Ring et al. 2012), especially N65. Specifically, the one or more substitutions are selected from the group consisting of N1D, N1A, N1G, N4D, S7Y, S7A, D8A, D8N, K10A, K11A, D30N, D61A, D61N, E64Q, N65D, N65A, N65E, N65R, N65K, L69R, N72R, Q101D, Q101E, Q108D, Q108A, Q108E, Q108R, preferably selected from the list consisting of D8A, D8N, D61A, D61N, N65A, N65D, N72R, Q101D, Q101E and Q108A more preferably selected from substitutions D61A (“DA” mutation), N65A (“NA” mutation), Q101D (“QD” mutation), especially N65A. N65K and L69R were reported to abrogate the binding of IL-2/IL-15Rβ (WO 2014/207173A1), whereas Q101D and Q108D to inhibit the function of IL-15 (WO 2006/020849A2) and are preferred substitutions. Q108D has specifically been described to increase affinity for CD122 and to impair recruitment of CD132 for inhibiting IL-2 and IL-15 effector functions, whereas N65K has been described to abrogate CD122 affinity (WO 2017/046200A1). N1D, N4D, D8N, D30N, D61N, E64Q, N65D, and Q108E were described for gradually reducing the activity of the respective IL-15/IL-15Rα conjugate regarding activating of NK cells and CD8 T cells (see FIG. 51, WO 2018/071918A1, WO 2018/071919A1). S7Y, S7A, K10A, K11A have been identified to reduce IL-2/IL-15Rβ binding (Ring et al. 2012).

Preferred combinations are D8N/N65A, D61A/N65A (“DANA” mutation), N1D/D61N, N1D/E64Q, N4D/D61N, N4D/E64Q, D8N/D61N, D8N/E64Q, D61N/E64Q, E64Q/Q108E, D61A/N65A/Q101D (“DANAQD” mutation), N1D/N4D/D8N, D61N/E64Q/N6SD (“NQD” mutation), N1D/D61N/E64Q, N1D/D61N/E64Q/Q108E, or N4D/D61N/E64Q/Q108E, more preferably D8N/N65A, D61A/N65A or D61A/N65A/Q101D, especially D61A/N65A.

A number of substitutions reducing the binding to the IL-2/IL-15Rβγ had been described in the prior art. However, suitable data on their effect on pharmacokinetics in mammals were missing and are widely unpredictable. The inventors identified a suitable range of IL-15 variants with the AQ mutation with additional single substitutions that markedly reduce the potency as tested in a fusion protein with the sushi+ fragment of IL-15Rα (RLI2). As shown in Table 11, the D61A substitution led to an about 8fold reduction, the N65D substitution to an about 20fold reduction and the N65A substitution to a 48fold reduction.

Similarly, immunocytokines based on the anti-PD-1 antibody pembrolizumab with an RLI2_{AQ} fused to the C-terminus of one or both heavy chains, or both light chains of the antibody were made (see example 11). The single substitutions again covered a range of reduced potency compared as EC50 on kit225 cells to wt RLI2 (set 100%). Whereas the fusion to the antibody already reduced the potency to about 50% (2 RLI2 molecules fused (x2)) or to about 15% (1 RLI2 (x1) molecule fused due to KIH technology) a range between about 40% to about 0.4% for the N65A substitution was observed. The NQD mutation had the lowest potency being below detection limit for the 1× molecule and about 0.04% for the x2 molecule in this assay. Further, immunocytokines based on the anti-PD-1 antibody pembrolizumab with an RLI2_{AQ} with mutations reducing the binding to IL-2Rβγ were fused to the light chains of the antibody were compared to respective immunocytokines, where the RLI2_{AQ} was fused to the C-terminus of one heavy chain of the antibody (see example 12). The homodimeric light chain fusions showed similar or lightly improved EC50 values compared to heterodimeric heavy chain fusions for the identical RLI2_{AQ} variants.

Compared to the RLI2_{AQ}NA (also named RLI-15_{AQA}) mutein, the QDQA (Q101D/Q108A) double substitution reduced potency on kit225 cells to about 50%, the NQD (D30N/E64Q/N65D) triple mutation to about 7% and the DANA (D61A/N65A) double substitution to about 1%.

The PEM-RLI NA x1 construct having a single RLI2_{AQ} NA fused to the a pembrolizumab derivative (see SEQ ID NO: 22, SEQ ID NO: 23 and SEQ ID NO: 24 but without the L235E substitution in the heavy chains) was shown to strongly decrease tumor volume in a murine tumor model in comparison to the control untreated group (p-value was <0.05) and similarly to the pembrolizumab treatment group (see example 14).

In a further embodiment the IL-15 variant comprises at least one further substitution that activates IL-15. Preferably, the activating mutation is at position N72, especially N72D. The AQ substitution may also be used to reduce heterogeneity in conjugates comprising an IL-15 variant having an activating mutation at position N72, as for example N72D as used in the clinical candidate IL-2/IL-15Rβγ agonist ALT-803.

In a further embodiment the IL-15 variant comprises at least one further substitution that reduces binding to the IL-15Rα, preferably the site for the amino acid substitution reducing binding to the IL-15Rα may be at one or more sites selected from the list consisting of L44, L45, E46, L47, V49, I50, S51, L52, E64, L66, I67, I68 or L69. Preferred are L44, E46, L47, V49, I50, S51, L66 and I67. The one or more substitutions are preferably selected from the list consisting of L44D, E46K, E46G, L47D, V49D, V49R, I50D, L66D, L66E, I67D, and I67E. L44D, E46K, L47D, V49D, I50D, L66D, L66E, I67D, and I67E were specifically described for reducing binding to the IL-15Rα (WO 2016/142314A1), whereas L45, S51 and/or L52 substituted by D, E, K or R and E64, I68 and L69 replaced by D, E, R or K to increase the binding to the IL-15Rα (WO 2005/085282A1). Similarly, IL-15 variants comprising amino acid substitutions at positions V49 and I51 or V49, I50 and S51, and further comprising one or more amino acids substitutions at positions N1, N4, S7, K10, K11, Y26, S29, D30, V31, H32, E53, G55, E64, I68, L69, E89, L91, M109, and/or I111 have been described to have decreased or no binding to IL-15Rα and the IL-2/IL-15βγ receptor

Preferred substitution combinations reducing binding to the IL-15Rα are E46G/V49R, N1A/D30N/E46G/V49R, N1G/D30N/E46G/V49R/E64Q, V49R/E46G/N1A/D30N and V49R/E46G/N1G/E64Q/D30N (WO 2019/166946A1). Similarly, amino acid sited L45, S51, L52, E64, 168, L69 have been described to reduce binding to the IL-15Rα. Preferably, L45, S51 and/or L52 are substituted with D, E, K or R, and E64, I68, L69 are substituted by D, E, R or K (WO 2005/085282A1).

In another embodiment additionally N71 is replaced by S, A or N, N72 by S, A or N, and N79 by S, A or G for reducing deamidation (WO 2009/135031A1).

WO 2016/060996A2 defines specific regions of IL-15 as being suitable for substitutions (see para. 0020, 0035, 00120 and 00130) and specifically provides guidance how to identify potential substitutions for providing an anchor for a PEG or other modifications (see para. 0021).

Additionally or alternatively, the artisan can easily make conservative amino acid substitutions.

In another aspect, the present invention relates to a conjugate comprising an IL-15 variant of the invention. IL-15 or IL-15 variants are used in various non-covalent or covalent conjugates in the clinic or at pre-clinical stage. RLI2/SO-C101/SOT101 (Cytune Pharma) is a covalent fusion protein of the sushi+ fragment of IL-15Rα, a linker and IL-15. NIZ985 (Novartis) is a heterodimeric, non-covalent conjugate of IL-15 with the soluble IL-15Rα. ALT-803 (Immunity-Bio/previously Altor) is a homodimeric non-covalent conjugate of two IL-15 N72D variants non-covalently bound to the IL-15Rα sushi domains, which are each N-terminally fused to IgG1-Fc chains. P-22339 (Hengrui Medicine) is a homodimeric covalent conjugate of two IL-15 variants bearing a cysteine substitution to form an artificial disulfide bridge linking the IL-15 variant to two IL-15Rα sushi domains also bearing a cysteine substitution, both being N-terminally fused to IgG-Fc chains. XmAb24306 (Xencor, Genentech) is a heterodimeric covalent conjugate of an IL-15 variant with reduced IL-2/IL-15Rβγ binding N-terminally fused to one Fc chain and an IL-15Rα sushi domain N-terminally fused to the other Fc chain. CUG105 (Cugene) is a heterodimeric covalent conjugate of an IL-15 N-terminally fused to one Fc chain and an IL-15Rα sushi domain N-terminally fused to the other Fc chain. Further, IL-15 or IL-15 variants are used as conjugates with PEG, e.g. AM0015 (Armo Bio, Eli Lilly), THOR-924, 908, 918 (Synthorx, Sanofi) or NKTR-255 (Nektar Therapeutics). It is foreseeable that the AQ mutation would similarly improve heterogeneity of such conjugates, as shown by the inventors for RLI2_{AQ} and RLI2_{AQ}-based immunocytokines.

In one embodiment, the conjugate further comprises the sushi domain of an IL-15Rα or a derivative thereof. Complexing the IL-15 with a sushi domain comprising polypeptide occupies the IL-15Rα binding site of IL-15 and therefore on the one hand abolishes binding to the IL-2/IL-15Rαβγ, increases binding affinity to the IL-2/IL-15Rβγ (compared to IL-15 alone) and circumvents the requirement of trans-presentation for IL-2/IL-15Rβγ expressing cells, thereby making such conjugate an IL-2/IL-15Rβγ superagonist. As described above, this concept is employed by a number of different approaches including RLI2/SO-C101/SOT101, NIZ985, ALT-803, P-22339, XmAb24306 and CUG105. Some only use the sushi domain, which is the minimal binding domain of the IL-15Rα to bind to IL-15 (e.g., ALT-803), some use the sushi+ fragment being an extended sushi domain with full binding activity to IL-15 (RLI2/SO-C101/SOT101), and other used the soluble IL-15Rα, i.e. the much larger polypeptide without its transmembrane domain (NIZ985). Derivatives of the sushi domain need to retain binding to IL-15 (retaining at least 25%, preferably at least 50% of the binding of the respective sushi domain), or within the conjugate block binding to the IL-2/IL15Rαβγ (i.e., reduce the binding affinity to the IL15Rαβγ by at least one log, preferably at least two logs). For example, WO 2016/095642 discloses sushi derivatives with a cysteine substitution at positions K34, L42, A37, G38, or S40 in order introduce an artificial disulfide bond with IL-15 variants having a cysteine substitution at L45, Q48, V49, L52, E53, C88 or E89, preferably the sushi S40C variant pairs with an IL-15 variant having the L52C substitution.

In another aspect of the invention, the present invention relates to a fusion protein comprising an IL-15 variant of the invention. Fusion proteins are preferred conjugates according to this invention, as compared to non-covalent conjugates there is no risk of dissociation of the conjugate after strong dilution of the conjugate upon administration into the patient. Also, typically expression of a fusion protein is more effective and leads to a more homogeneous product than co-expression of multiple polypeptide chains, or even in vitro assembly of polypeptides after individual purification. Fusion proteins comprising an IL-15 variant fused to the C-terminus of a heavy chain of an antibody are for example disclosed in WO 2019/166946A1 (Pfizer) or WO 2018/184964A1 (Roche), or comprising an IL-15 variant fused to each C-terminus of the heavy chains of an antibody are for example disclosed in WO 2016/142314A1 (DKFZ, Univ. Tubingen).

In one embodiment, the fusion protein of the invention further comprises the sushi domain of an IL-15Rα or derivative thereof, a targeting moiety, and/or a half-life extending moiety, and optionally one or more linker(s). As stated above, fusions with the sushi domain of an IL-15Rα or a derivative thereof are preferred as the resulting fusion protein has an optimized targeting to the IL-2/IL-15Rβγ with no binding to the IL-15Rαβγ and with no need for trans presentation of the IL-15Rα. Further, the IL-15 variant may be fused to a targeting moiety. Targeting moieties are primarily antibodies or functional fragments binding to the same target thereof and the IL-15 or an IL-15/IL-15Rα fusion protein may be preferably fused to the C-terminus of one or both heavy chains (to one heavy chain requiring a heterodimerization mutation in the Fc domain such as the KiH technology), or to both light chains. Other targeting moieties may be short binding tags, such as an RGD motif (see e.g. WO 2017/000913), the albumin binding domain (ABD) (see e.g. WO 2018/151868A2), TCRs (see e.g. WO 2008/143794) or antibody mimetics such as anticalins, affibodies, adectins, aptamers, affimers, affitins, avimers, fynomers, armadillo repeat proteins, and knottins (Yu et al. 2017). The IL-15 variant may also be fused to half-life extending moieties, such as an Fc domain or human serum albumin. It is a common strategy in IL-15 developments to increase the in vivo half-life to extend the stimulation of reactive immune cells, primarily NK and CD8⁺ T cells by increasing the size of the protein and thereby slowing down clearance from the blood stream. The fusion to an Fc domain has been employed for example in the development candidates P-22339, XmAb24306 and CUG105.

In a preferred embodiment, the fusion protein of the invention comprises, preferably in N- to C-terminal order, the human IL-15Rα sushi domain, a linker and the IL-15 variant of the invention. The order receptor-linker-interleukin (“RLI”) was shown to be beneficial compared to the opposite order ILR. Preferably the human IL-15Rα sushi domain comprises the sequence of SEQ ID NO: 5, wherein the linker has a length of 18 to 22 amino acids and is composed of glycines or serines and glycines, and an IL-15 variant of the invention. Human sequences are preferred for human patients. A linker of a length of 18 to 22 amino acids had been shown to be beneficial and glycines or serines and glycines are amino acids preferred for the linker sequence to make the linker flexible and non-immunogenic. RLI2/SO-C101/SOT101 is a clinical stage fusion protein with the sushi+ fragment of the IL-15Rα, which is improved to have a superior homogeneity by introducing the AQ substitution. Accordingly, RLI2_{AQ} (SEQ ID NO: 9) is a preferred embodiment. Another preferred RLI molecule having a less potent IL-15 variant is RLIAQ N65A/RLI-15_{AQA} (SEQ ID NO: 10). Generally, used linkers are composed of glycines or serines and glycines and have a length of 10 to 40 amino acids.

In another embodiment, the targeting moiety is an antibody or functional variant thereof that preferably binds to a tumor antigen, a tumor extracellular matrix antigen, or a tumor neovascularization antigen, or is an immunomodulatory antibody.

Tumor antigens are preferably selected from EGFR, HER2, FGFR2, FOLR1, CLDN18.2, CEA, GD2, O-Acetyl-GD-2, GM1, CAIX, EPCAM, MUC1, PSMA, c-Met, CD19, CD20, CD38. Tumor extracellular matrix antigens are preferably selected from FAP, the EDA domain of fibronectin, the EDB domain of fibronectin and LRRC15, preferably FAP and the EDB domain of fibronectin.

Neovascularization antigens are preferably selected from VEGF, or Endoglin; (CD105).

An immunomodulatory antibody or a functional variant thereof may be an immunomodulatory antibody which stimulates a co-stimulatory receptor, preferably selected from CD40 agonists, CD137/4-1BB agonists, CD134/OX40 agonists and TNFRSF18/GITR agonists, or the immunomodulatory antibody may inhibit an immunosuppressive receptor, preferably selected from PD-1 antagonists, CTLA-4 antagonists, LAG3 antagonists, TIGIT antagonists, inhibitory KIRs antagonists, BTLA/CD272 antagonists, HAVCR2/TIM-3/CD366 antagonists and ADORA2A antagonists, more preferably PD-1 antagonists.

Antibodies against the listed targets above are well known in the art or can be generated by standard immunization or phage display protocols. Non-human antibodies can be humanized. Examples of anti-EGFR antibodies are cetuximab, panitumumab, zalutumumab, nimotuzumab, and matuzumab. Examples of anti-HER2 antibodies are trastuzumab, permtuzumab or margetuximab. Examples of anti-CLDN18.2 antibodies are zolbetuximab and antibodies of the invention below. An example of an anti-CEA antibody is arcitumomab. An example of an anti-GD2 is hu14.18K322A. An example of an anti O-Acetyl-GD-2 is c.8B6. Examples of anti-CD20 antibodies are rituximab, ocrelizumab, obinutuzu-mab, ofatumumab, ibritumomab, tositumomab and ublituximab. Examples of anti-CD38 antibodies are daratumumab, MOR202 and isatuximab.

Examples anti-FAP antibodies are Sibrotuzumab and B12 (US 2020-0246383A1). An example of an anti-EDA domain antibody of fibronectin is the F8 antibody ((Villa et al. 2008), WO 2010/078945, WO 2014/174105), an example of an anti-EDB domain of fibronectin is the L19 antibody ((Pini et al. 1998), WO 1999/058570), and an example of an anti-LRRC15 antibody is Samrotamab/huM25 (WO 2017/095805).

Examples of anti-VEGF antibodies are bevacizumab and ranibizumab. An example of an anti-Endoglin antibody is TRC 105 (WO 2010039873A2).

Examples of anti-CD40 agonistic antibodies are selicrelumab, APX005M, ChiLob7/4, ADC-1013, SEA-CD40 and CDX-1140 (Vonderheide 2020). Examples of anti-CD137/4-1BB agonistic antibodies are urelumab and utomilumab (Chester et al. 2018). Examples of anti-CD134/OX40 agonistic antibodies PF-04518600, MED16469, MOXR0916, MED10562, INCAGN01949 (Fu et al. 2020). An example of an anti-TNFRSF18/GITR agonistic antibody is DTA-1.

Examples of PD-1 antagonists are anti-PD-1 antibodies, anti-PD-L1 antibodies or anti-PD-L2 antibodies Examples of anti-PD-1 antagonistic antibodies are pembrolizumab, nivolumab, pidilizumab, toripalimab and tislelizumab (Dolgin 2020). Examples of anti-PD-L1 antagonistic antibodies are atezolizumab and avelumab. An example of an anti-CTLA-4 antagonistic antibody is ipilimumab. An example of an anti-LAG3 antagonistic antibody is relatlimab, Examples of anti-TIGIT antagonistic antibodies are Tiragolumab, Vibostolimab, Domvanalimab, Etigilimab, BMS-986207, EOS-448, COM902, ASP8374, SEA-TGT, BGB-A1217, IBI-939 and M6223 (Dolgin 2020).

An example of an anti-BTLA antagonistic antibody is TAB004. Examples of anti-HAVCR2/TIM-3 antagonistic antibodies are LY3321367, MBG453 and TSR-022.

In a preferred embodiment, the fusion protein is fused to the C-terminus of at least one heavy chain of the antibody or to the C-terminus of both light chains of the antibody. Various immunocytokines, i.e. antibodies fused to a cytokine, were made and tested in examples 7 to 14 by fusing RLI2_{AQ} without a linker to the C-terminus of one (e.g. SEQ ID NO: 22) or both heavy chains (e.g. SEQ ID NO: 25), or to both light chains (e.g. SEQ ID NO: 30) of a pembrolizumab-derived antibody. Alternatively, a linker may be used for fusing RLI2_{AQ} to the C-terminus of one or both heavy chains. Such linker is preferably composed of glycins or glycins and serins, more preferably composed of GGGGS units with a length of 30 to 50 amino acids, especially the L40 linker of SEQ ID NO: 31. An exemplary immunocytokine base on the anti-CD20 antibody having RLI2AQ fused to both heavy chains with the L40 linker was made (SEQ ID NO: 32, SEQ ID NO: 34). For generating heterodimeric immunocytokines having one RLI molecule fused to one heavy chain, the KiH technology with the T366W mutation in one chain (knob) and the T366S/L368A/Y407V in the other chain (hole) was applied (Elliott et al. 2014). Other heterodimerization technologies are known in the art, e.g. KiH_{S-S} (T366W/S354C-T366S/L368A/Y407V/Y349C, (Merchant et al. 1998, Leaver-Fay et al. 2016)), HA-TF (S364H/F405A-Y349T/T394F, (Moore et al. 2011)), ZW1 (T350V/L351Y/F405A/Y407V-T350V/T366L/K392L/T394W, (Von Kreudenstein et al. 2013)), 7.8.60 (K360D/D399M/Y407A-E345R/Q347R/T366V/K409V, (Leaver-Fay et al. 2016)), DD-KK (K409D/K392D-D399K/E356K, (Gunasekaran et al. 2010)), EW-RVT (K360E/K409W-Q347R/D399V/F405T, (Choi et al. 2013, Choi et al. 2015)), EW-RVTS-S(K360E/K409W/Y349C-Q347R/D399V/F405T/S354C, (Choi et al. 2015)). SEED f(IgA-derived 45 residues on IgG1 CH3-IgG1-derived 57 residues on IgA CH3, (Davis et al. 2010)), A107 (K370E/K409W-E357N/D399V/F405T, (Choi et al. 2015)). The IgG4 based Fc domains of the immunocytokines were modified by the L235E mutation to further reduce the ADCC activity (Alegre et al. 1992) and/or by the M252Y/S254T/T256E mutation to increase FcRn binding for extending the in vivo half-life (Dall'Acqua et al. 2002). In another embodiment, the antibody targeting a check point inhibitor such as PD-1 or CTLA-4 may be in the IgG1 format engineered to have strongly reduced or silenced ADCC and/or CDC activity, e.g., having reduced FcγR and C1q binding. Suitable Fc modifications for immunocytokines are listed in Table 2.

TABLE 2
Examples of modifications to modulate antibody effector function. Unless otherwise noted,
the mutations are on the IgG1 subclass. Adapted from Wang et al. (Wang et al. 2018).

| Engineering and intended | | |
|---|---|---|
| function | Mutation | Reference |
| Enhance ADCC | | |
| Increased FcγRIIIa binding | F243L/R292P/Y300L/V305I/P396L | (Stavenhagen et al. 2007) |
| function | S239D/I332E | (Lazar et al. 2006) |
| | S298A/E333A/K334A | (Shields et al. 2001) |
| | K392T/P396L | WO2006/088494 |
| | V264I/I332E | WO2004/099249 |
| | P247L/D270E/N241K | WO2008/140603 |
| | P247I/A339Q | (Forero-Torres et al. 2012) |
| | in one heavy chain: | (Mimoto et al. 2013) |
| | L234Y/L235Q/G236W/S239M/H2- | (Nordstrom et al. 2011) |
| | 68D/D270E/S298A, | |
| | in the opposing heavy chain: | |
| | D270E/K326D/A330M/K334E | |
| | in the light chain: N65S, | |
| | in the heavy chain: | |
| | L235V/F243L/R292P/Y300L/P39-6L | |
| Increased FcγRIIIa | S239D/A330L/I332E | (Lazar et al. 2006) |
| binding, decreased FcγRIIb | | |
| binding | | |
| Enhance ADCP | | |
| Increased FcγRIIa binding, | G236A/S239D/I332E | (Richards et al. 2008) |
| Increased FcγRIIIa binding | G236A/S239D/A330L/I332E | (Ahmed et al. 2016) |
| Enhance CDC | | |
| Increased C1q binding | K326W/E333S | (Idusogie et al. 2001) |
| | S267E/H268F/S324T | (Moore et al. 2010) |
| | IgG1/IgG3 cross subclass | (Natsume et al. 2008) |
| Hexamerization | E345R/E430G/S440Y | (Diebolder et al. 2014) |
| Reduce effector function | | |
| Aglycosylated | N297A or N297Q or N297G | (Tao and Morrison 1989, |
| | | Walker et al. 1989, Bolt et |
| | | al. 1993, Leabman et al. |
| | | 2013) |
| Reduced FcγR and | IgG1: L234A/L235A or | (Xu et al. 2000, Lo et al. |
| C1q binding | L234A/L235A/P329G | 2017) |
| | IgG4: L235E | (Alegre et al. 1992) |
| | IgG4: F234A/L235A | (Xu et al. 2000) |
| | E233P/F234V/L235A/D265A/L309V/R409K | (Zhang et al. 2018) |
| | IgG2/IgG4 cross isotype | (Rother et al. 2007) |
| | IgG2: H268Q/V309L/A330S/P331S | (An et al. 2009) |
| | IgG2: | (Vafa et al. 2014) |
| | V234A/G237A/P238S/H268A/V309L/A330S/P331S | |
| Increase half-life | | |
| Increased FcRn binding | M252Y/S254T/T256E | (Dall'Acqua et al. 2002) |
| binding at pH 6.0 | M428L/N434S | (Zalevsky et al. 2010) |
| Increased co-engagement | | |
| Increased FcγRIIb binding | S267E/L328F | (Chu et al. 2008) |
| Increased FcγRIIa binding, | N325S/L328F | (Shang et al. 2014) |
| decreased FcγRIIIa binding | | |

Different IL-15 variants (all having the AQ mutation) with further mutations reducing the IL-2Rβγ binding were used in the RLI conjugates.

The N65A substitution of IL-15 was identified as a single mutation tuning down the RLI-15 activity to a level suitable for many antibodies. Accordingly, the fusion proteins comprising RLI-15_{AQA} are preferred embodiments of the invention.

One preferred embodiment is the fusion protein targeted to PD-1 comprises the sequence of and the antibody comprising the pembrolizumab-derived heavy chain knob sequence of SEQ ID NO: 22 (fused to SEQ ID NO: 10), the pembrolizumab-derived heavy chain hole sequence of SEQ ID NO: 23, and the light chain sequence of SEQ ID NO: 24, wherein the conjugate is fused to the C-terminus heavy chain knob sequence without a linker. In a more preferred embodiment, the fusion protein targeted to PD-1 comprises the antibody comprising SEQ ID NO: 22, SEQ ID NO: 38 and SEQ ID NO: 24 (SOT201).

One preferred embodiment is the conjugate of the sequence SEQ ID NO: 10 and the anti-CLDN18.2 heterodimeric IgG1 antibody variant having a VH and VL domain sequence of SEQ ID NO: 46 and SEQ ID NO: 47, respectively, the IgG1 variant being heterodimeric through the KiH mutation (T366W mutation in one chain (knob) and the T366S/L368A/Y407V in the other chain (hole). In a preferred embodiment the conjugate comprises the SEQ ID NO: 66, SEQ ID NO: 67 and SEQ ID NO: 68 (SOT202).

Further embodiments are all polypeptides comprising an IL-15 variant listed in Table 1.

In another aspect of the invention, the invention relates to a nucleic acid encoding the IL-15 variant of the invention, the conjugate of the invention, or the fusion protein of the invention.

Further, one aspect of the invention relates to a vector comprising the nucleic acid of the invention.

Further, one aspect of the invention relates to a host cell comprising the nucleic acid of the invention or the vector of the invention.

Another aspect of the invention relates to the IL-15 variant of the invention, the conjugate of the invention, or the fusion protein of any of the invention, the nucleic acid of the invention or the vector of the invention for use in treatment. IL-15 and accordingly IL-15 variants of the invention are powerful cytokines used and/or tested clinically or preclinically as medicinal products for the treatment of neoplastic diseases (Robinson and Schluns 2017) and infectious diseases.

Another aspect of the invention relates to a pharmaceutical composition comprising the IL-15 variant of the invention, the conjugate of the invention, or the fusion protein of the invention, the nucleic acid of the invention or the vector of the invention and a pharmaceutically acceptable carrier. Additionally, the pharmaceutical composition may comprise pharmaceutically acceptable excipients such as detergents, salts and/or cryoprotectives.

Yet another aspect of the invention relates to the IL-15 variant of the invention, the conjugate of the invention, or the fusion protein of the invention, the nucleic acid of the invention or the vector of the invention for use in the treatment of a subject suffering from, at risk of developing and/or being diagnosed for a neoplastic disease or an infectious disease.

In one embodiment, the neoplastic disease is selected from solid tumor or hematological diseases. Examples of solid tumors are colorectal cancer, gastric cancer, melanoma, ocular melanoma, Merkel-cell carcinoma, skin squamous-cell carcinoma, anal cancer, renal cell carcinoma, bladder cancer, adenocarcinoma, carcinoid tumor, leiomyosarcoma, breast cancer, triple-negative breast cancer, osteosarcoma, thyroid cancer, thymic cancer, cholangiocarcinoma, salivary gland cancer, adenoid cystic carcinoma, gastric cancer, head and neck squamous cell carcinoma, non-small cell lung cancer, small-cell lung cancer, hepatocellular carcinoma, ovarian cancer, cervical cancer, biliary tract cancer, urothelial cancer and mesothelioma. In one embodiment microsatellite instability high solid tumors are preferred. Examples of hematological cancers are leukemias such as acute lymphoblastic leukemia (ALL), acute myelogenous leukemia (AML), chronic lymphocytic leukemia (CLL), Chronic myelogenous leukemia (CML) and acute monocytic leukemia (AMoL), lymphomas such as Hodkin's lymphomas, Non-Hodgkin's lymphomas, and myelomas. In one embodiment, the infectious disease is selected from HIV, hepatitis A, B or C, and herpes virus infections.

In one aspect, the present invention relates to a method of treating a subject, wherein the method comprises administering the IL-15 variant of the invention, the conjugate of the invention, or the fusion protein of the invention, the nucleic acid of the invention or the vector of the invention in a therapeutically effective amount to the subject in need thereof.

In one embodiment, the present invention relates to a polypeptide comprising an amino acid sequence according to SEQ ID NO: 9.

In another embodiment, the present invention relates to a polypeptide comprising an amino acid sequence according to SEQ ID NO: 10.

### FIGURES

FIG. 1: (A) LMW SDS-PAGE and Western-blot (anti-RLI-15) analysis of RLI2 (RLI2 wt), RLI2 with G78A substitution (RLI2 A) and RLI2 with G78A/N79Q substitutions (RLI2 AQ) under non-reducing conditions. For Coomassie staining 0.5 μg or 2 μg or protein were used (lanes 2, 4, 6, 8, 10 and 12) and for Western blotting 25 ng of protein were used (lanes 3, 7, 11).

(B) Capillary Electrophoresis, denaturing, analysis of RLI2 (RLI2 wt), RLI2 with G78A substitution (RLI2 A) and RLI2 with G78A/N79Q substitutions (RLI2 AQ) under reducing (R) and non-reducing (NR) conditions. Dotted box 1 represents band for glycosylation site #2 (main), box 2 represents band for glycosylation site #1 (minor), dotted box 3 represents new glycosylation site for RLI2 A. Non-named lanes are marker with 16, 21, 30, 48 and 68 kDa.

FIG. 2: Analysis of the 3 deglycosylated RLI variants expressed in CHO cells by SDS-PAGE (7.5-18%) stained by Coomassie blue (left pane), by silver nitrate (middle pane) and detected by an anti-IL15 western blot (right pane): lanes 1: molecular weight marker; lanes 2: RLI2_{N176Q}, lanes 3: RLI2_{N168S/N176Q/N209S}, lanes 4: RLI1_{N168S/N176Q/N209S}.

FIG. 3: Potency of RLI2 and RLI2_{AQ} from supernatants determined by activation of 32Db cells or Kit225 cells. (A) 32Db cells, 21 h, (B) Kit225 cells, 4 h.

FIG. 4: Relative potency of RLI2 purified or from supernatant compared to RLI2_{AQ} from supernatant determined by activation of Kit225 cells.

FIG. 5: Comparison of highly glycosylated RLI2 and low glycosylated RLI2 (A) CPI HIC elution profile in dependence of Concentration of Buffer B measured at 280 nm. Left box indicates pooled fraction 2B11-3 for highly glycosylated RLI2 (“RLI-15-HG”) and right box indicates pooled fractions 4B11-3 for low glycosylated RLI2 (“RLI-15-LG”). (B) SDS PAGE of fractions 2B1 1-3 of RLI-15-HG, RLI2 reference standards and molecular weight ladders of given kDa. (C) SDS PAGE of fraction 4B1 1-3 of RLI-15-LG, RLI2 reference standards and molecular weight ladders of given kDa.

FIG. 6: In vitro mixed lymphocyte reaction (hPBMC donors): relative IFNγ production is shown for PEM (pembrolizumab) and RLI-15 (RLI2) compared to immunocytokine PEM LY-RLI NA x1 (IL-15 N65A mutant also having AQ mutation).

FIG. 7: In vivo hPD1 single KI HuGEMM mice implanted with HuCell MC38-hPD-L1 tumor cell line was used as an animal tumor model. Tumor volume is shown for control (triangles), pembrolizumab (grey circles) dosed at DO, D3, D6 and D9 with 5 mg/kg and PEM-RLI NA x1 (black circles) dosed at D0 with 20 mg/kg.

FIG. 8: Comparison of ADCC activity of immunocytokines based on the hCl1a antibody with non-modified effector functions to immunocytokines with reduced ADCC activity and antibodies hCl1a and Zolbetuximab. ADCC target cells were A549 cells overexpressing CLDN18.2 (A549-CLDN18.2) or PA-TU-8988S cells (PATU) endogenously expressing CLDN18.2.

FIG. 9: Comparison of ADCC activity of immunocytokines based on the hCl1a antibody with non-modified effector functions to immunocytokines with enhanced ADCC activity and antiodies hCl1a and Zolbetuximab. ADCC target cells were A549 cells overexpressing CLDN18.2 (A549-CLDN18.2) or PA-TU-8988S cells (PATU) endogenously expressing CLDN18.2. (A): DLE mutation; (B): DE mutation; (C): AAA muation; (D): TL mutation; (E): IE mutation; (F): afucosylated immunocytokines.

FIG. 10: (A) % of PD-1/PD-L1 blocking is shown in dependence of increasing concentrations in pM of Keytruda and SOT201

(B) % of Ki67⁺ NK cells and CD8⁺ T cells determined by flow cytometry after 7 days stimulation in vitro of human PBMC from healthy donors with increasing amounts of SOT201 or SOT201 wt having an IL-15 moiety without reduced binding to the IL-2/IL-15Rβγ.

(C) Cell proliferation (Ki67⁺) of CD8⁺ T cells or NK cells detected in spleen of healthy C57BL/6 mice (n=2/group) by flow cytometry 5 days after IV injection of compounds at equimolar amount to 5 mg/kg of the murine surrogate molecule mSOT201 (anti-murine PD-1 antibody RMP1-14 fused RLI-15_{AQA}) compared to the anti-murine PD-1 antibody alone or to the anti-human PD1 mouse IgG1-RLI-15_{AQA} (hPD1-mSOT201) as single activity controls.

FIG. 11: (A) Tumor volume in mm³ over a time course of 17 days of C57BL/6 mice bearing syngeneic MC38 tumor cells treated IV with a single injection of control (NaCl, mSOT201, hPD1-mSOT201 or mPD1 at equimolar amount to mSOT201 (5 mg/kg) on day 1 (randomization day of tumor volumes 80-100 mm³) (n=10 mice/group).

(B) corresponding % of surviving MC38 tumor bearing mice up to 100 days post treatment.

FIG. 12: (A) Relative expression levels of gene sets associated with indicated adaptive and innate immune cells and cancer associated fibroblasts (CAFs) across mSOT201 treated tumor samples (N=3) and control samples (n=4) of MC38 tumor bearing mice as determined by metagenes on RNA seq data. Box plots: minimum, median, maximum.

(B) Cell proliferation as determined by % Ki67⁺ cells by flow cytometry of indicated cells in spleen or lymph nodes in MC38 tumor bearing mice on day 7 after mSOT201 (5 mg/kg) IV treatment of established tumors (80-100 mm³) (n=2).

FIG. 13: (A) Tumor volume in mm³ over a time course of 21 days of C57BL/6 mice bearing MC38 tumors treated IV with a single injection of control (NaCl), mSOT201, the mPD1-IL-2βγ agonist (IL-2v fused to the anti-murinePD-1 antibody RMP1-14) with abolished CD25 binding or the combination of RLI-15_{AQA} with the anti-murine PD-1 antibody mPD1 (RMP1-14) (n=10 mice/group).

(B) Cell proliferation as determined by % Ki67⁺ cells of CD8⁺ T cells and NK cells detected by flow cytometry after IV administration in healthy C57/BL6 mice at day 5 and day 8.

(C) % Ki67⁺ cells of CD8⁺ T cells in spleen or lymph nodes at day 7 of C57BL/6 mice bearing MC38 tumors treated IV with mSOT201, mPD1-IL-2v or the combination of RLI-15_{AQA} and mPD-1. Randomization day 1, tumor volumes 100 mm³ (n=10/group).

FIG. 14: (A) % of Ki67+ and fold change of absolute cell counts of NK and CD8⁺ T cells in blood of cynomolgus monkeys after a single IV administration of 0.6 mg/kg of SOT201 at day 1 determined at indicated days by flow cytometry and haematology, each graph curve representing one animal.

(B) % of Ki67⁺ of NK and CD8⁺ T cells in blood of cynomolgus monkeys after administration on days 1 and 21 (indicated by arrows) IV administration of 0.3 mg/kg of SOT201 determined at indicated days by flow cytometry, each graph curve representing one animal.

FIG. 15: NK and CD8⁺ T cell proliferation upon treatment with mouse SOT201 surrogates in vivo.

(A) Proliferation of CD8⁺ T cells and NK cells in spleen of healthy C57BL/6 mice at day 5 and 8 after treatment with hPD1-mSOT201, mPD-1, mSOT201, mSOT201 wt and mPD1-IL2v. The expression of Ki67 in CD8⁺ T cells and NK cells was detected by flow cytometry. The molecules were administered i.v. on day 1 at doses equimolar to 5 mg/kg of mSOT201: hPD1-mSOT201 at 5.37 mg/kg, mPD-1 at 4.51 mg/kg, and at a dose equimolar to 0.25 mg/kg of mSOT201 wt: mPD1-IL2v at 0.26 mg/kg. Flow cytometry analysis was performed on day 5 and day 8. The data represent mean SEM for 2 individuals per group per day.

(B) Proliferation of CD8⁺ T cells and NK cells in spleen of healthy C57BL/6 mice at day 5 and 8 after treatment with hPD1-mSOT201, mPD-1, mSOT201, mSOT201 wt and mPD1-IL2v. The expression of Ki67 in CD8⁺ T cells and NK cells was detected by flow cytometry. The molecules were administered i.v. on day 1 at doses equimolar to 10 mg/kg of mSOT201: hPD1-mSOT201 at 10.74 mg/kg, mPD-1 at 9.02 mg/kg, and at dose equimolar to 0.1 mg/kg of mSOT201 wt: mPD1-IL2v at 0.1 mg/kg. Flow cytometry analysis was performed on day 5 and day 8. The data represent mean±SEM for 2 individuals per group per day.

FIG. 16: mouse SOT201 surrogates in PD-1 sensitive and PD-1 resistant tumor models in vivo.

(A) Anti-PD-1 sensitive tumor models

MC38/C57BL/6 mouse model: single i.v. administration at Day 0 of 4.51 mg/kg mPD-1 (sub-optimal dose as compared to literature, selected as equimolar to mSOT201), 5 mg/kg mSOT201 or 5.37 mg/kg hPD1-mSOT201 (equimolar to mSOT201); D0=randomization day with tumor volume of ˜80-100 mm³, 10 mice/group;

CT26/BALB/c mouse model: four i.p. administrations at Day 0, 3, 6 and 9 with 9.02 mg/kg mPD-1 (effective dose as compared to literature), 10 mg/kg mSOT201, 10.74 mg/kg hPD1-mSOT201 (equimolar to mSOT201); D0=randomization day with tumor volume of ˜100 mm³, 10 mice/group.

(B) Anti-PD-1 resistant tumor models

CT26 STK11 ko mouse model: four i.p. administrations at Day 0, 3, 6 and 9 with 9.02 mg/kg mPD-1 (effective dose as compared to literature), 10 mg/kg mSOT201, 10.74 mg/kg hPD1-mSOT201 (equimolar to mSOT201); D0=randomization day with tumor volume of ˜100 mm³, 10 mice/group. B16F10/C57BL/6 mouse model: four i.p. administrations at Day 0, 3, 6 and 9 with 9.02 mg/kg mPD-1 (effective dose as compared to literature), 10 mg/kg mSOT201, 10.74 mg/kg hPD1-mSOT201 (equimolar to mSOT201); Day 0=randomization day with tumor volume of ˜100 mm³, 10 mice/group Cut-off day for all mice present in the control groups, CR=complete response.

FIG. 17: Comparison of mSOT201 vs. RLI-15_{AQA} mutein+anti-PD-1 in vivo.

MC38/C57BL/6 mouse model with following groups:

G1 mock control

G4: a single administration of 0.64 mg/kg RLI-15_{AQA}, s.c. at Day 0+a single administration of 4.51 mg/kg mPD-1, i.p. at Day 0.

G2 a single administration of 5 mg/kg mSOT201, i.v. at Day 0

G3 a single administration of 2 mg/kg mSOT201, i.v. at Day 0

G6 a single administration of 4.51 mg/kg single mPD1, i.p. at Day 0 (suboptimal dose as compared to literature, selected as equimolar to mSOT201),

G11 a single administration of 5 mg/kg hPD1-mSOT201, i.v. at Day 0+a single administration of 4.36 mg/kg mPD-1, i.p. at Day 0,

Day 0=randomization day with tumor volume of ˜80-100 mm³, 10 mice/group

Cut-off day for all mice present in the control groups, CR=complete response.

FIG. 18: MC38/C57BL/6 mouse model—D0=randomization day ˜80-100 mm³, 10 mice/group. CR=complete response

G1 mock control

G2 single administration of 5 mg/kg of mSOT201, i.v. at Day 0

G3 single administration of 2 mg/kg of mSOT201, i.v. at Day 0

G7 4 administrations of 1 mg/kg of RLI2_{AQ}, s.c. at Day 0, 1, 2 and 3

G5 single administration of 1 mg/kg RLI2_{AQ}, s.c. at Day 0+single administration of 5 mg/kg mPD1, i.p. at Day 0

G8 4 administrations of 1 mg/kg of RLI2_{AQ}, s.c. at Day 0, 1, 2 and 3+single administration of 5 mg/kg mPD1, i.p. at Day 0

G9 4 administrations of 1 mg/kg of RLI2_{AQ}, s.c. at Day 0, 1, 2, and 3+4 administrations of 5 mg/kg mPD1, i.p. at Day 0, 3, 6 and 9

G6 single administration of 5 mg/kg mPD1, i.p. at Day 0

G10 4 administrations of 5 mg/kg mPD1, i.p. at Day 0, 3, 6 and 9

Cut-off day for all mice present in the control groups

FIG. 19: Comparison of mSOT201 vs. RLI2_{AQ}+anti-PD-1 tumor growth in vivo. MC38/C57BL/6 mouse model

(A) average tumor volume in mm³ in dependence of time and shown for individual animals at day 16, with the horizonal line showing the mean tumor volume.

G1 mock control

G2 single administration of 2 mg/kg of mSOT201, i.v. at Day 0,

G3 two administration of 2 mg/kg RLI2_{AQ}, s.c. at Day 0 and 1+4 administrations of 2 mg/kg mPD1, i.p. at Day 0, 3, 6 and 9.

1 experiment only, D0=randomization day at tumor volume of ˜80-100 mm³, 10 mice/group.

CR=complete response

The relative expansion of NK cells, CD8⁺ T cells and cells expressing αβTCR and γδTCR (T cells) was investigated in spleen, lymph nodes and tumor at day 7 after SOT201 (G2 from above) and RLI2_{AQ}+anti-PD-1 (G3 from above) treatment using flow cytometry. 3 tumor samples were pooled and 3 spleen and lymph node samples were analyzed separately.

(B) Frequency of parent (relative percentage compared to parent population) in % is shown for CD8⁺ T cells (top row) and NK cells (bottom row) from lymph nodes, spleen and tumor.

(C) Frequency of parent in % is shown for αβTCR⁺CD3⁺ T cells (top row) and βγTCR⁺CD3⁺ T cells (bottom row) from lymph nodes, spleen and tumor.

FIG. 20: (A) Immunogenicity in DC-T cell-based assay. T cell response to PEM-RLI-15 candidate molecules shown as % CFSE^{low} stained CD4⁺ T cells after loading of iDCs with candidate molecules, incubation with autologous CD4⁺ T cells pre-stained with CFSE and detection of CFSE staining with CFSE^{low} as a surrogate for cycling cells. Mean of 11 donors±SEM is shown. Significant differences compared to control DCs incubated with no protein and thus inducing non-specific T cell proliferation are shown. * p≤0.05, *** p≤0.001.

(B) FluoroSpot assay for IFN-γ and TNF-α of RLI-15 peptides spanning the introduced substitutions N65A and G175A/N176Q. Estimation of the effect of Mut2 or Mut3 peptides vs. respective wildtype peptides on the average dSFU in the test population of 40 donors with 95% confidence intervals (CI). SFU=Spot-forming Units, dSFU=SFU of restimulated well minus SFU of non-restimulated well.

FIG. 21: Comparison of the capacity to induce proliferation of hPBMCs of SOT202 molecules with modified effector functions. Proliferation of isolated hPBMC was assessed for SOT202-DANA, SOT202-afuc-DANA, SOT202-DLE-DANA, SOT202-DE-DANA and SOT202-LALAPG-DANA. Cells were stimulated in vitro for 7 days. Mean of 6 donors±SEM is shown. Proliferation of NK (top) and CD8⁺ T cells (bottom) was measured by counting Ki67⁺ cells by flow cytometry.

FIG. 22: Comparison of the capacity to induce proliferation of hPBMCs of SOT202 molecules and SOT201. Proliferation of isolated hPBMC was assessed for SOT202, SOT202-afuc, SOT201-DANA, SOT202-DANA and SOT202-afuc-DANA. Proliferation of NK (top) and CD8⁺ T cells (bottom) was measured by counting Ki67⁺ cells by flow cytometry.

FIG. 23: Comparison of the capacity to induce proliferation of hPBMCs of SOT202-DANA molecules with modified effector functions and SOT201-DANA. Proliferation of isolated hPBMC was assessed for SOT201-DANA, SOT202-DANA, SOT202-afuc-DANA, SOT202-LALAPG-DANA and hCl1a (also labelled SOT202-mab). Proliferation of NK (top) and CD8⁺ T cells (bottom) was measured by counting Ki67⁺ cells by flow cytometry.

FIG. 24: (A) Cell proliferation (Ki67+) of CD8⁺ T cells or NK cells detected in spleen of healthy C57BL/6 mice after stimulation with mSOT202. Cell proliferation was detected by Ki67 staining and measured by flow cytometry 5 days after IV injection of compounds of mSOT202 (hCl1a-mIgG2a-NA 1x) at 5, 10 or 20 mg/kg or of hCl1a-mIgG2a.

(B) Percentage of NK cell and CD8⁺ T cell under the same experimental conditions as in (A).

FIG. 25: Cell proliferation of NK cells (A) or CD8⁺ T cells (B) detected in spleen of healthy C57BL/6 mice after stimulation with mSOT202, mSOT202-LALAPG and hCl1a-mIgG2a. Top: Cell proliferation was detected by Ki67 staining and measured by flow cytometry 5 and 10 days after IV injection of the compounds at 5 mg/kg. Bottom: Percentage of NK cell and CD8⁺ T cell.

Sequences

SEQ ID NO: 1: human IL-15

| | | |
|---|---|---|
| 1 | MRISKPHLRS ISIQCYLCLL LNSHFLTEAG IHVFILGCFS AGLPKTEANW VNVISDLKKI | |
| 061 | EDLIQSMHID ATLYTESDVH PSCKVTAMKC FLLELQVISL ESGDASIHDT VENLIILANN | |
| 121 | SLSSNGNVTE SGCKECEELE EKNIKEFLQS FVHIVQMFIN TS | 162 |

Signal peptide underlined
SEQ ID NO: 2: mature human IL-15

| | | |
|---|---|---|
| 1 | NWVNVISDLK KIEDLIQSMH IDATLYTESD VHPSCKVTAM KCFLLELQVI SLESGDASIH | |
| 061 | DTVENLIILA NNSLSSNGNV TESGCKECEE LEEKNIKEFL QSFVHIVQMF INTS | 114 |

with G78 and N79 bold/underlined
SEQ ID NO: 3: mature human IL-15_{AQ}

| | | |
|---|---|---|
| 1 | NWVNVISDLK KIEDLIQSMH IDATLYTESD VHPSCKVTAM KCFLLELQVI SLESGDASIH | |
| 061 | DTVENLIILA NNSLSSNAQV TESGCKECEE LEEKNIKEFL QSFVHIVQMF INTS | 114 |

with A78 and Q79 bold/underlined
SEQ ID NO: 4: human IL-15Rα

| | | |
|---|---|---|
| 1 | MAPRRARGCR TLGLPALLLL LLLRPPATRG ITCPPPMSVE HADIWVKSYS LYSRERYICN | |
| 61 | SGFKRKAGTS SLTECVLNKA TNVAHWTTPS LKCIRDPALV HQRPAPPSTV TTAGVTPQPE | |
| 121 | SLSPSGKEPA ASSPSSNNTA ATTAAIVPGS QLMPSKSPST GTTEISSHES SHGTPSQTTA | |
| 181 | KNWELTASAS HQPPGVYPQG HSDTTVAIST STVLLCGLSA VSLLACYLKS RQTPPLASVE | |
| 241 | MEAMEALPVT WGTSSRDEDL ENCSHHL | 267 |

SEQ ID NO: 5: sushi domain of IL-15Rα

| | | |
|---|---|---|
| 001 | CPPPMSVEHA DIWVKSYSLY SRERYICNSG FKRKAGTSSL TECVLNKATN VAHWTTPSLK | 60 |
| 061 | C | |

SEQ ID NO: 6: sushi+ fragment of IL-15Rα

| | | |
|---|---|---|
| 001 | ITCPPPMSVE HADIWVKSYS LYSRERYICN SGFKRKAGTS SLTECVLNKA TNVAHWTTPS | |
| 061 | LKCIRDPALV HORPAPP | 77 |

SEQ ID NO: 7: linker

| | | |
|---|---|---|
| 001 | SGGSGGGGSG GGSGGGGSGG | 20 |

SEQ ID NO: 8: RLI2 (or SO-C101, SOT101)

| | | |
|---|---|---|
| 001 | ITCPPPMSVE HADIWVKSYS LYSRERYICN SGFKRKAGTS SLTECVLNKA TNVAHWTTPS | |
| 061 | LKCIRDPALV HQRPAPPSGG SGGGGSGGGS GGGGSGGNWV NVISDLKKIE DLIQSMHIDA | |
| 121 | TLYTESDVHP SCKVTAMKCF LLELQVISLE SGDASIHDTV ENLIILANNS LSSNGNVTES | |
| 181 | GCKECEELEE KNIKEFLQSF VHIVQMFINT S | 211 |

SEQ ID NO: 9: RLI2_{AQ}

| | | |
|---|---|---|
| 001 | ITCPPPMSVE HADIWVKSYS LYSRERYICN SGFKRKAGTS SLTECVLNKA TNVAHWTTPS | |
| 061 | LKCIRDPALV HQRPAPPSGG SGGGGSGGGS GGGGSGGNWV NVISDLKKIE DLIQSMHIDA | |
| 121 | TLYTESDVHP SCKVTAMKCF LLELQVISLE SGDASIHDTV ENLIILANNS LSSNAQVTES | |
| 181 | GCKECEELEE KNIKEFLQSF VHIVQMFINT S | 211 |

SEQ ID NO: 10: RLI2_{AQ}N162A (N65A) or RLI-15_{AQA}

| | | |
|---|---|---|
| 001 | ITCPPPMSVE HADIWVKSYS LYSRERYICN SGFKRKAGTS SLTECVLNKA TNVAHWTTPS | |
| 061 | LKCIRDPALV HQRPAPPSGG SGGGGSGGGS GGGGSGGNWV NVISDLKKIE DLIQSMHIDA | |
| 121 | TLYTESDVHP SCKVTAMKCF LLELQVISLE SGDASIHDTV EALIILANNS LSSNAQVTES | |
| 181 | GCKECEELEE KNIKEFLQSF VHIVQMFINT S | 211 |

SEQ ID NO: 11: Leader peptide of (IL-15_{N72D})₂:IL-15Rαₛᵤₛₕᵢ-FC:

| | | |
|---|---|---|
| 001 | METDTLLLWV LLLWVPGSTG | 20 |

SEQ ID NO: 12: IL-15Rαₛᵤₛₕᵢ (65 aa)-Fc (IgG1 CH2-CH3):

| | | |
|---|---|---|
| 1 | ITCPPPMSVE HADIWVKSYS LYSRERYICN SGFKRKAGTS SLTECVLNKA TNVAHWTTPS | |
| 61 | LKCIREPKSC DKTHTCPPCP APELLGGPSV FLFPPKPKDT LMISRTPEVT CVVVDVSHED | |
| 121 | PEVKFNWYVD GVEVHNAKTK PREEQYNSTY RVVSVLTVLH QDWLNGKEYK CKVSNKALPA | |
| 181 | PIEKTISKAK GQPREPQVYT LPPSRDELTK NQVSLTCLVK GFYPSDIAVE WESNGQPENN | |
| 241 | YKTTPPVLDS DGSFFLYSKL TVDKSRWQQG NVFSCSVMHE ALHNHYTQKS LSLSPGK | 297 |

SEQ ID NO: 13: IL-15_{N72D}

| | | |
|---|---|---|
| | NW VNVISDLKKI | |
| 061 | EDLIQSMHID ATLYTESDVH PSCKVTAMKC FLLELQVISL ESGDASIHDT VENLIILAND | |
| 121 | SLSSNGNVTE SGCKECEELE EKNIKEFLQS FVHIVQMFIN TS | 162 |

SEQ ID NO: 14: pembrolizumab heavy chain (HC)-human IgG4 κ isotype

| | | |
|---|---|---|
| 001 | QVQLVQSGVE VKKPGASVKV SCKASGYTFT NYYMYWVRQA PGQGLEWMGG INPSNGGTNF | 060 |
| 061 | NEKFKNRVTL TTDSSTTTAY MELKSLQFDD TAVYYCARRD YRFDMGFDYW GQGTTVTVSS | 120 |
| 121 | ASTKGPSVFP LAPCSRSTSE STAALGCLVK DYFPEPVTVS WNSGALTSGV HTFPAVLQSS | 180 |
| 181 | GLYSLSSVVT VPSSSLGTKT YTCNVDHKPS NTKVDKRVES KYGPPCPPCP APEFLGGPSV | 240 |
| 241 | FLFPPKPKDT LMISRTPEVT CVVVDVSQED PEVQFNWYVD GVEVHNAKTK PREEQFNSTY | 300 |
| 301 | RVVSVLTVLH QDWLNGKEYK CKVSNKGLPS SIEKTISKAK GQPREPQVYT LPPSQEEMTK | 360 |
| 361 | NQVSLTCLVK GFYPSDIAVE WESNGQPENN YKTTPPVLDS DGSFFLYSRL TVDKSRWQEG | 420 |
| 421 | NVFSCSVMHE ALHNHYTQKS LSLSLG | |

The pembrolizumab HC has stabilizing S228P mutation; for immunocytokines herein,
terminal K has been deleted to reduce heterogeneity.
SEQ ID NO: 15: pembrolizumab HC CDR1

| | |
|---|---|
| 001 | NYYMY |

SEQ ID NO: 16: pembrolizumab HC CDR2

| | |
|---|---|
| 001 | GINPSNGGTNFNEKFKN |

SEQ ID NO: 17: pembrolizumab HC CDR3

| | |
|---|---|
| 001 | RDYRFDMGFDY |

SEQ ID NO: 18: pembrolizumab light chain

| | | |
|---|---|---|
| 001 | EIVLTQSPAT LSLSPGERAT LSCRASKGVS TSGYSYLHWY QQKPGQAPRL LIYLASYLES | 060 |
| 061 | GVPARFSGSG SGTDFTLTIS SLEPEDFAVY YCQHSRDLPL TFGGGTKVEI KRTVAAPSVE | 120 |
| 121 | IFPPSDEQLK SGTASVVCLL NNFYPREAKV QWKVDNALQS GNSQESVTEQ DSKDSTYSLS | 180 |
| 181 | STLTLSKADY EKHKVYACEV THQGLSSPVT KSFNRGEC | |

SEQ ID NO: 19: pembrolizumab LC CDR1

| | |
|---|---|
| 001 | RASKGVSTSGYSYLH |

SEQ ID NO: 20: pembrolizumab LC CDR2

| | |
|---|---|
| 001 | LASYLES |

SEQ ID NO: 21: pembrolizumab LC CDR3

| | |
|---|---|
| 001 | QHSRDLPLT |

SEQ ID NO: 22: SOT201 HC knob: IgG4 S228P.L235E.T366W.dK-RLI2.N162A.G175A.N176Q

| | | |
|---|---|---|
| 001 | QVQLVQSGVE VKKPGASVKV SCKASGYTFT NYYMYWVRQA PGQGLEWMGG INPSNGGTNF | 060 |
| 061 | NEKFKNRVTL TTDSSTTTAY MELKSLQFDD TAVYYCARRD YRFDMGFDYW GQGTTVTVSS | 120 |
| 121 | ASTKGPSVFP LAPCSRSTSE STAALGCLVK DYFPEPVTVS WNSGALTSGV HTFPAVLQSS | 180 |
| 181 | GLYSLSSVVT VPSSSLGTKT YTCNVDHKPS NTKVDKRVES KYGPPCPPCP APEFEGGPSV | 240 |
| 241 | FLFPPKPKDT LMISRTPEVT CVVVDVSQED PEVQFNWYVD GVEVHNAKTK PREEQFNSTY | 300 |
| 301 | RVVSVLTVLH QDWLNGKEYK CKVSNKGLPS SIEKTISKAK GQPREPQVYT LPPSQEEMTK | 360 |
| 361 | NQVSLWCLVK GFYPSDIAVE WESNGQPENN YKTTPPVLDS DGSFFLYSRL TVDKSRWQEG | 420 |
| 421 | NVFSCSVMHE ALHNHYTQKS LSLSLGITCP PPMSVEHADI WVKSYSLYSR ERYICNSGFK | 480 |
| 481 | RKAGTSSLTE CVLNKATNVA HWTTPSLKCI RDPALVHQRP APPSGGSGGG GSGGGSGGGG | 540 |
| 541 | SGGNWVNVIS DLKKIEDLIQ SMHIDATLYT ESDVHPSCKV TAMKCFLLEL QVISLESGDA | 600 |
| 601 | SIHDTVEALI ILANNSLSSN AQVTESGCKE CEELEEKNIK EFLQSFVHIV QMFINTS | 657 |

SEQ ID NO: 23: pembrolizumab variant HC hole: S228P.L235E.T366S.L368A.Y407V

| | | |
|---|---|---|
| 001 | QVQLVQSGVE VKKPGASVKV SCKASGYTFT NYYMYWVRQA PGQGLEWMGG INPSNGGTNF | 060 |
| 061 | NEKFKNRVTL TTDSSTTTAY MELKSLQFDD TAVYYCARRD YRFDMGFDYW GQGTTVTVSS | 120 |
| 121 | ASTKGPSVFP LAPCSRSTSE STAALGCLVK DYFPEPVTVS WNSGALTSGV HTFPAVLQSS | 180 |
| 181 | GLYSLSSVVT VPSSSLGTKT YTCNVDHKPS NTKVDKRVES KYGPPCPPCP APEFEGGPSV | 240 |
| 241 | FLFPPKPKDT LMISRTPEVT CVVVDVSQED PEVQFNWYVD GVEVHNAKTK PREEQFNSTY | 300 |
| 301 | RVVSVLTVLH QDWINGKEYK CKVSNKGLPS SIEKTISKAK GQPREPQVYT LPPSQEEMTK | 360 |
| 361 | NQVSLSCAVK GFYPSDIAVE WESNGQPENN YKTTPPVLDS DGSFFLVSRL TVDKSRWQEG | 420 |
| 421 | NVFSCSVMHE ALHNHYTQKS LSLSLGK | |

SEQ ID NO: 24: SOT201 LC

| | | |
|---|---|---|
| 001 | EIVLTQSPAT LSLSPGERAT LSCRASKGVS TSGYSYLHWY QQKPGOAPRL LIYLASYLES | 060 |
| 061 | GVPARFSGSG SGTDFTLTIS SLEPEDFAVY YCQHSRDLPL TFGGGTKVEI KRTVAAPSVE | 120 |
| 121 | IFPPSDEQLK SGTASVVCLL NNFYPREAKV QWKVDNALQS GNSQESVTEQ DSKDSTYSLS | 180 |
| 181 | STLTLSKADY EKHKVYACEV THQGLSSPVT KSFNRGEC | 240 |

SEQ ID NO: 25: pembrolizumab heavy chain (HC)-human IgG4 κ-RLI2 AQ

| | | |
|---|---|---|
| 001 | QVQLVQSGVE VKKPGASVKV SCKASGYTFT NYYMYWVRQA PGQGLEWMGG INPSNGGTNF | 060 |
| 061 | NEKFKNRVTL TTDSSTTTAY MELKSLQFDD TAVYYCARRD YRFDMGFDYW GQGTTVTVSS | 120 |
| 121 | ASTKGPSVFP LAPCSRSTSE STAALGCLVK DYFPEPVTVS WNSGALTSGV HTFPAVLQSS | 180 |
| 181 | GLYSLSSVVT VPSSSLGTKT YTCNVDHKPS NTKVDKRVES KYGPPCPPCP APEFLGGPSV | 240 |
| 241 | FLFPPKPKDT LMISRTPEVT CVVVDVSQED PEVQFNWYVD GVEVHNAKTK PREEQFNSTY | 300 |
| 301 | RVVSVLTVLH QDWLNGKEYK CKVSNKGLPS SIEKTISKAK GQPREPQVYT LPPSQEEMTK | 360 |
| 361 | NQVSLTCLVK GFYPSDIAVE WESNGQPENN YKTTPPVLDS DGSFFLYSRL TVDKSRWQEG | 420 |
| 421 | NVFSCSVMHE ALHNHYTQKS LSLSLGITCP PPMSVEHADI WVKSYSLYSR ERYICNSGFK | 480 |
| 481 | RKAGTSSLTE CVLNKATNVA HWTTPSLKCI RDPALVHQRP APPSGGSGGG GSGGGSGGGG | 540 |
| 541 | SGGNWVNVIS DLKKIEDLIQ SMHIDATLYT ESDVHPSCKV TAMKCFLLEL QVISLESGDA | 600 |
| 601 | SIHDTVENLI ILANNSLSSN AQVTESGCKE CEELEEKNIK EFLQSFVHIV QMFINTS | 657 |

SEQ ID NO: 26: IgG4 Fc KiH-knob
APEFLGGPSVFLFPPKPKDTLMISRTPEVTCVVVDVSQEDPEVQFNWYVDGVEVHNAKTKPREEQFNSTYRVVSV
LTVLHQDWLNGKEYKCKVSNKGLPSSIEKTISKAKGQPREPQVYTLPPSQEEMTKNQVSLWCLVKGFYPSDIAVE
WESNGQPENNYKTTPPVLDSDGSFFLYSRLTVDKSRWQEGNVFSCSVMHEALHNHYTQKSLSLSLGK
SEQ ID NO: 27: IgG4 Fc KiH-hole
APEFLGGPSVFLFPPKPKDTLMISRTPEVTCVVVDVSQEDPEVQFNWYVDGVEVHNAKTKPREEQFNSTYRVVSV
LTVLHQDWLNGKEYKCKVSNKGLPSSIEKTISKAKGQPREPQVYTLPPSQEEMTKNQVSLSCAVKGFYPSDIAVE
WESNGQPENNYKTTPPVLDSDGSFFLVSRLTVDKSRWQEGNVFSCSVMHEALHNHYTQKSLSLSLGK
SEQ ID NO: 28: IgG4 Fc LE (L235E)
APEFEGGPSVFLFPPKPKDTLMISRTPEVTCVVVDVSQEDPEVQFNWYVDGVEVHNAKTKPREEQFNSTYRVVSV
LTVLHQDWLNGKEYKCKVSNKGLPSSIEKTISKAKGQPREPQVYTLPPSQEEMTKNQVSLTCLVKGFYPSDIAVE
WESNGQPENNYKTTPPVLDSDGSFFLYSRLTVDKSRWQEGNVFSCSVMHEALHNHYTQKSLSLSLGK
SEQ ID NO: 29: CL domain of LC κ-RLI2 AQ
RTVAAPSVFIFPPSDEQLKSGTASVVCLLNNFYPREAKVQWKVDNALQSGNSQESVTEQDSKDSTYSLSSTLTLS
KADYEKHKVYACEVTHQGLSSPVTKSFNRGECITCPPPMSVEHADIWVKSYSLYSRERYICNSGFKRKAGTSSLT
ECVLNKATNVAHWTTPSLKCIRDPALVHQRPAPPSGGSGGGGSGGGSGGGGSGGNWVNVISDLKKIEDLIQSMHI
DATLYTESDVHPSCKVTAMKCFLLELQVISLESGDASIHDTVENLIILANNSLSSNAQVTESGCKECEELEEKNI
KEFLQSFVHIVQMFINTS
SEQ ID NO: 30: SOT201 LC-RLI2 AQ

| | | |
|---|---|---|
| 001 | EIVLTQSPAT LSLSPGERAT LSCRASKGVS TSGYSYLHWY QQKPGQAPRL LIYLASYLES | 060 |
| 061 | GVPARFSGSG SGTDFTLTIS SLEPEDFAVY YCQHSRDLPL TFGGGTKVEI KRTVAAPSVE | 120 |
| 121 | IFPPSDEQLK SGTASVVCLL NNFYPREAKV QWKVDNALQS GNSQESVTEQ DSKDSTYSLS | 180 |
| 181 | STLTLSKADY EKHKVYACEV THQGLSSPVT KSENRGECIT CPPPMSVEHA DIWVKSYSLY | 240 |
| 241 | SRERYICNSG FKRKAGTSSL TECVLNKATN VAHWTTPSLK CIRDPALVHQ RPAPPSGGSG | 300 |
| 301 | GGGSGGGSGG GGSGGNWVNV ISDLKKIEDL IQSMHIDATL YTESDVHPSC KVTAMKCELL | 360 |
| 361 | ELQVISLESG DASIHDTVEN LIILANNSLS SNAQVTESGC KECEELEEKN IKEFLQSFVH | 420 |
| 421 | IVQMFINTS | 429 |

SEQ ID NO: 31: L40 Linker

| | | |
|---|---|---|
| 001 | GGGGSGGGGS GGGGSGGGGS GGGGSGGGGS GGGGSGGGGS | 040 |

SEQ ID NO: 32: RTX HC-L40-RLI2_{AQ}
QVQLQQPGAELVKPGASVKMSCKASGYTFTSYNMHWVKQTPGRGLEWIGAIYPGNGDTSYNQKFKGKATLT
ADKSSSTAYMQLSSLTSEDSAVYYCARSTYYGGDWYFNVWGAGTTVTVSAASTKGPSVFPLAPSSKSTSGG
TAALGCLVKDYFPEPVTVSWNSGALTSGVHTFPAVLQSSGLYSLSSVVTVPSSSLGTQTYICNVNHKPSNT
KVDKKVEPKSCDKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTVDKSRWQQGNV
FSCSVMHEALHNHYTQKSLSLSPGGGGGSGGGGSGGGGSGGGGSGGGGSGGGGSGGGGSGGGGSITCPPPM
SVEHADIWVKSYSLYSRERYICNSGFKRKAGTSSLTECVLNKATNVAHWTTPSLKCIRDPALVHQRPAPPS
GGSGGGGSGGGSGGGGSGGNWVNVISDLKKIEDLIQSMHIDATLYTESDVHPSCKVTAMKCFLLELQVISL
ESGDASIHDTVENLIILANNSLSSNGNVTESGCKECEELEEKNIKEFLQSFVHIVQMFINTS
SEQ ID NO: 33: RTX HC-RLI2AQ
QVQLQQPGAELVKPGASVKMSCKASGYTFTSYNMHWVKQTPGRGLEWIGAIYPGNGDTSYNQKFKGKATLT
ADKSSSTAYMQLSSLTSEDSAVYYCARSTYYGGDWYFNVWGAGTTVTVSAASTKGPSVFPLAPSSKSTSGG
TAALGCLVKDYFPEPVTVSWNSGALTSGVHTFPAVLQSSGLYSLSSVVTVPSSSLGTQTYICNVNHKPSNT
KVDKKVEPKSCDKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTVDKSRWQQGNV
FSCSVMHEALHNHYTQKSLSLSPGITCPPPMSVEHADIWVKSYSLYSRERYICNSGFKRKAGTSSLTECVL
NKATNVAHWTTPSLKCIRDPALVHQRPAPPSGGSGGGGSGGGSGGGGSGGNWVNVISDLKKIEDLIQSMHI
DATLYTESDVHPSCKVTAMKCFLLELQVISLESGDASIHDTVENLIILANNSLSSNAQVTESGCKECEELE
EKNIKEFLQSFVHIVQMFINTS
SEQ ID NO: 34: RTX LC
QIVLSQSPAILSASPGEKVTMTCRASSSVSYIHWFQQKPGSSPKPWIYATSNLASGVPVRFSGSGSGTSYS
LTISRVEAEDAATYYCQQWTSNPPTFGGGTKLEIKRTVAAPSVFIFPPSDEQLKSGTASVVCLLNNFYPRE
AKVQWKVDNALQSGNSQESVTEQDSKDSTYSLSSTLTLSKADYEKHKVYACEVTHQGLSSPVTKSFNRGEC
SEQ ID NO: 35: hCl1a HC AAA Knob RLI2 NA
QVQLVQSGAEVKKPGASVKVSCKASGYTFTDYAMHWVRQAPGQRLEWMGWINTYTGKPTYAQKFQGRVTITRDTS
ASTAYMELSSLRSEDTAVYYCARAVFYGYTMDAWGQGTLVTVSSASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKKVEPKSCDKT
HTCPPCPAPELLGGPSVFLFPPKPKDTLMISRTPEVTCVVVDVSHEDPEVKFNWYVDGVEVHNAKTKPREEQYNA
TYRVVSVLTVLHQDWLNGKEYKCKVSNKALPAPIAATISKAKGQPREPQVYTLPPSRDELTKNQVSLWCLVKGFY
PSDIAVEWESNGQPENNYKTTPPVLDSDGSFFLYSKLTVDKSRWQQGNVFSCSVMHEALHNHYTQKSLSLSPGIT
CPPPMSVEHADIWVKSYSLYSRERYICNSGFKRKAGTSSLTECVLNKATNVAHWTTPSLKCIRDPALVHQRPAPP
SGGSGGGGSGGGSGGGGSGGNWVNVISDLKKIEDLIQSMHIDATLYTESDVHPSCKVTAMKCELLELQVISLESG
DASIHDTVEALIILANNSLSSNAQVTESGCKECEELEE KNIKEFLQSFVHIVQMFINTS
SEQ ID NO: 36: hCl1a HC AAA Hole
QVQLVQSGAEVKKPGASVKVSCKASGYTFTDYAMHWVRQAPGQRLEWMGWINTYTGKPTYAQKFQGRVTITRDTS
ASTAYMELSSLRSEDTAVYYCARAVFYGYTMDAWGQGTLVTVSSASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKKVEPKSCDKT
HTCPPCPAPELLGGPSVFLFPPKPKDTLMISRTPEVTCVVVDVSHEDPEVKFNWYVDGVEVHNAKTKPREEQYNA
TYRVVSVLTVLHQDWLNGKEYKCKVSNKALPAPIAATISKAKGQPREPQVYTLPPSRDELTKNQVSLSCAVKGFY
PSDIAVEWESNGQPENNYKTTPPVLDSDGSFFLVSKLTVDKSRWQQGNVFSCSVMHEALHNHYTQKSLSLSPG
SEQ ID NO: 37: hCl1a LC
DIQMTQSPSSLSASVGDRVTITCRASEDIYSNLAWYQQKPGKAPKLLIFSVKRLQDGVPSRFSGSGSGTDFTLTI
SSLQPEDFATYYCLQGSNFPLTFGQGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSFNRGEC
SEQ ID NO: 38: SOT201 HC hole: S228P.L235E.T366S.L368A.Y407V/dK

| | | |
|---|---|---|
| 001 | QVQLVQSGVE VKKPGASVKV SCKASGYTFT NYYMYWVRQA PGQGLEWMGG INPSNGGTNF | 060 |
| 061 | NEKFKNRVTL TTDSSTTTAY MELKSLQFDD TAVYYCARRD YRFDMGFDYW GQGTTVTVSS | 120 |
| 121 | ASTKGPSVFP LAPCSRSTSE STAALGCLVK DYFPEPVTVS WNSGALTSGV HTFPAVLQSS | 180 |
| 181 | GLYSLSSVVT VPSSSLGTKT YTCNVDHKPS NTKVDKRVES KYGPPCPPCP APEFEGGPSV | 240 |
| 241 | FLFPPKPKDT LMISRTPEVT CVVVDVSQED PEVQFNWYVD GVEVHNAKTK PREEQFNSTY | 300 |
| 301 | RVVSVLTVLH QDWLNGKEYK CKVSNKGLPS SIEKTISKAK GQPREPQVYT LPPSQEEMTK | 360 |
| 361 | NQVSLSCAVK GFYPSDIAVE WESNGQPENN YKTTPPVLDS DGSFFLVSRL TVDKSRWQEG | 420 |
| 421 | NVFSCSVMHE ALHNHYTQKS LSLSLG | |

SEQ ID NO: 39: mPD1.VH-h1.HC.D265A.E356K.N399K.dk-RLI.N162A.G175A.N176Q
murine antiPD-1 (mIgG1 D265A HC1-RLI-15AQA)
EVQLQESGPGLVKPSQSLSLTCSVTGYSITSSYRWNWIRKFPGNRLEWMGYINSAGISNYNPSLKRRISITRDTS
KNQFFLQVNSVTTEDAATYYCARSDNMGTTPFTYWGQGTLVTVSSAKTTPPSVYPLAPGSAAQTNSMVTLGCLVK
GYFPEPVTVTWNSGSLSSGVHTFPAVLQSDLYTLSSSVTVPSSTWPSQTVTCNVAHPASSTKVDKKIVPRDCGCK
PCICTVPEVSSVFIFPPKPKDVLTITLTPKVTCVVVAISKDDPEVQFSWFVDDVEVHTAQTKPREEQINSTFRSV
SELPIMHQDWLNGKEFKCRVNSAAFGAPIEKTISKTKGRPKAPQVYTIPPPKKQMAKDKVSLTCMITNFFPEDIT
VEWQWNGQPAENYKNTQPIMKTDGSYFVYSKLNVQKSNWEAGNTFTCSVLHEGLHNHHTEKSLSHSPITCPPPMS
VEHADIWVKSYSLYSRERYICNSGFKRKAGTSSLTECVLNKATNVAHWTTPSLKCIRDPALVHQRPAPPSGGSGG
GGSGGGSGGGGSGGNWVNVISDLKKIEDLIQSMHIDATLYTESDVHPSCKVTAMKCFLLELQVISLESGDASIHD
TVEALIILANNSLSSNAQVTESGCKECEELEEKNIKEFLQSFVHIVQMFINTS
SEQ ID NO: 40: mPD1.VH-h1.HC.D265A.K409E.K439D.dk
murine antiPD-1 (mIgG1 D265A HC2)
EVQLQESGPGLVKPSQSLSLTCSVTGYSITSSYRWNWIRKFPGNRLEWMGYINSAGISNYNPSLKRRISITRDTS
KNQFFLQVNSVTTEDAATYYCARSDNMGTTPFTYWGQGTLVTVSSAKTTPPSVYPLAPGSAAQTNSMVTLGCLVK
GYFPEPVTVTWNSGSLSSGVHTFPAVLQSDLYTLSSSVTVPSSTWPSQTVTCNVAHPASSTKVDKKIVPRDCGCK
PCICTVPEVSSVFIFPPKPKDVLTITLTPKVTCVVVAISKDDPEVQFSWFVDDVEVHTAQTKPREEQINSTFRSV
SELPIMHQDWLNGKEFKCRVNSAAFGAPIEKTISKTKGRPKAPQVYTIPPPKEQMAKDKVSLTCMITNFFPEDIT
VEWQWNGQPAENYKNTQPIMNTDGSYFVYSELNVQKSNWEAGNTFTCSVLHEGLHNHHTEDSLSHSP
SEQ ID NO: 41: mPD1.VL-hk.LC
murine antiPD-1 (mlgG1 Light Chain)
DIVMTQGTLPNPVPSGESVSITCRSSKSLLYSDGKTYLNWYLQRPGQSPQLLIYWMSTRASGVSDRFSGSGSGTD
FTLKISGVEAEDVGIYYCQQGLEFPTFGGGTKLELKRTDAAPTVSIFPPSSEQLTSGGASVVCFLNNFYPKDINV
KWKIDGSERQNGVLNSWTDQDSKDSTYSMSSTLTLTKDEYERHNSYTCEATHKTSTSPIVKSFNRNEC
SEQ ID NO: 42: human IL-2

| | | |
|---|---|---|
| 001 | APTSSSTKKT QLQLEHLLLD LQMILNGINN YKNPKLTRML TFKFYMPKKA TELKHLQCLE | 060 |
| 061 | EELKPLEEVL NLAQSKNFHL RPRDLISNIN VIVLELKGSE TTFMCEYADE TATIVEFLNR | 120 |
| 121 | WITFCQSIIS TLT | 133 |

SEQ ID NO: 43: IL-2v

| | | |
|---|---|---|
| 1 | APASSSTKKT QLQLEHLLLD LQMILNGINN YKNPKLTRML TAKFAMPKKA TELKHLQCLE | 060 |
| 061 | EELKPLEEVL NGAQSKNFHL RPRDLISNIN VIVLELKGSE TTFMCEYADE TATIVEFLNR | 120 |
| 121 | WITFAQSIIS TLT | |

SEQ ID NO: 44: IL-15 M1

| | | |
|---|---|---|
| 001 | AWVNVISDLK KIEDLIQSMH IDATLYTESN VHPSCKVTAM KCFLLGLQRI SLESGDASIH | 060 |
| 061 | DTVENLIILA NNSLSSNGNV TESGCKECEE LEEKNIKEFL QSFVHIVQMF INTS | 114 |

SEQ ID NO: 45: IL-15 M2

| | | |
|---|---|---|
| 001 | GWVNVISDLK KIEDLIQSMH IDATLYTESN VHPSCKVTAM KCFLLGLQRI SLESGDASIH | 060 |
| 061 | DTVQNLIILA NNSLSSNGNV TESGCKECEE LEEKNIKEFL QSFVHIVQMF INTS | 114 |

SEQ ID NO: 46: hCl1a VH
QVQLVQSGAEVKKPGASVKVSCKASGYTFTDYAMHWVRQAPGQRLEWMGWINTYTGKPTYAQKFQGRVTITRDTS
ASTAYMELSSLRSEDTAVYYCARAVFYGYTMDAWGQGTLVTVSS
SEQ ID NO: 47: hCl1a VL
DIQMTQSPSSLSASVGDRVTITCRASEDIYSNLAWYQQKPGKAPKLLIFSVKRLQDGVPSRFSGSGSGTDFTLTI
SSLQPEDFATYYCLQGSNFPLTFGQGTKVEIK
SEQ ID NO: 48: hCl1a HC Knob
QVQLVQSGAEVKKPGASVKVSCKASGYTFTDYAMHWVRQAPGQRLEWMGWINTYTGKPTYAQKFQGRVTITRDTS
ASTAYMELSSLRSEDTAVYYCARAVFYGYTMDAWGQGTLVTVSSASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKKVEPKSCDKT
HTCPPCPAPELLGGPSVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSRDELTKNQVSLWCLVKGFY
PSDIAVEWESNGQPENNYKTTPPVLDSDGSFFLYSKLTVDKSRWQQGNVFSCSVMHEALHNHYTQKSLSLSPG
SEQ ID NO: 49: hCl1a HC Hole
QVQLVQSGAEVKKPGASVKVSCKASGYTFTDYAMHWVRQAPGQRLEWMGWINTYTGKPTYAQKFQGRVTITRDTS
ASTAYMELSSLRSEDTAVYYCARAVFYGYTMDAWGQGTLVTVSSASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKKVEPKSCDKT
HTCPPCPAPELLGGPSVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSRDELTKNQVSLSCAVKGFY
PSDIAVEWESNGQPENNYKTTPPVLDSDGSFFLVSKLTVDKSRWQQGNVFSCSVMHEALHNHYTQKSLSLSPG
SEQ ID NO: 50: hCl1a HC Knob RLI2 NA
QVQLVQSGAEVKKPGASVKVSCKASGYTFTDYAMHWVRQAPGQRLEWMGWINTYTGKPTYAQKFQGRVTITRDTS
ASTAYMELSSLRSEDTAVYYCARAVFYGYTMDAWGQGTLVTVSSASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKKVEPKSCDKT
HTCPPCPAPELLGGPSVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSRDELTKNQVSLWCLVKGFY
PSDIAVEWESNGQPENNYKTTPPVLDSDGSFFLYSKLTVDKSRWQQGNVFSCSVMHEALHNHYTQKSLSLSPGIT
CPPPMSVEHADIWVKSYSLYSRERYICNSGFKRKAGTSSLTECVLNKATNVAHWTTPSLKCIRDPALVHQRPAPP
SGGSGGGGSGGGSGGGGSGGNWVNVISDLKKIEDLIQSMHIDATLYTESDVHPSCKVTAMKCFLLELQVISLESG
DASIHDTVEALIILANNSLSSNAQVTESGCKECEELEEKNIKEFLQSFVHIVQMFINTS
SEQ ID NO: 51: PD1-IL2v HC1: HC with IL2v (Fc knob, LALAPG),
IL2v.T3A.F42A.Y45A.L72G.C125A
EVQLLESGGGLVQPGGSLRLSCAASGFSFSSYTMSWVRQAPGKGLEWVATISGGGRDIYYPDSVKGRFTISRDNS
KNTLYLQMNSLRAEDTAVYYCVLLTGRVYFALDSWGQGTLVTVSSASTKGPSVFPLAPSSKSTSGGTAALGCLVK
DYFPEPVTVSWNSGALTSGVHTFPAVLQSSGLYSLSSVVTVPSSSLGTQTYICNVNHKPSNTKVDKKVEPKSCDK
THTCPPCPAPEAAGGPSVFLFPPKPKDTLMISRTPEVTCVVVDVSHEDPEVKFNWYVDGVEVHNAKTKPREEQYN
STYRVVSVLTVLHQDWLNGKEYKCKVSNKALGAPIEKTISKAKGQPREPQVYTLPPCRDELTKNQVSLWCLVKGF
YPSDIAVEWESNGQPENNYKTTPPVLDSDGSFFLYSKLTVDKSRWQQGNVFSCSVMHEALHNHYTQKSLSLSPGG
GGGSGGGGSGGGGSAPASSSTKKTQLQLEHLLLDLQMILNGINNYKNPKLTRMLTAKFAMPKKATELKHLQCLEE
ELKPLEEVLNGAQSKNFHLRPRDLISNINVIVLELKGSETTFMCEYADETATIVEFLNRWITFAQSIISTLT
SEQ ID NO: 52: PD1-IL2v HC2: HC (Fc hole LALAPG)
EVQLLESGGGLVQPGGSLRLSCAASGFSFSSYTMSWVRQAPGKGLEWVATISGGGRDIYYPDSVKGRFTISRDNS
KNTLYLQMNSLRAEDTAVYYCVLLTGRVYFALDSWGQGTLVTVSSASTKGPSVFPLAPSSKSTSGGTAALGCLVK
DYFPEPVTVSWNSGALTSGVHTFPAVLQSSGLYSLSSVVTVPSSSLGTQTYICNVNHKPSNTKVDKKVEPKSCDK
THTCPPCPAPEAAGGPSVFLFPPKPKDTLMISRTPEVTCVVVDVSHEDPEVKFNWYVDGVEVHNAKTKPREEQYN
STYRVVSVLTVLHQDWLNGKEYKCKVSNKALGAPIEKTISKAKGQPREPQVCTLPPSRDELTKNQVSLSCAVKGF
YPSDIAVEWESNGQPENNYKTTPPVLDSDGSFFLVSKLTVDKSRWQQGNVFSCSVMHEALHNHYTQKSLSLSP
SEQ ID NO: 53: PD1-IL2v LC
DIVMTQSPDSLAVSLGERATINCKASESVDTSDNSFIHWYQQKPGQSPKLLIYRSSTLESGVPDRFSGSGSGTDF
TLTISSLQAEDVAVYYCQQNYDVPWTFGQGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEVTHQGLSSPVTKSFNRGEC
SEQ ID NO: 54: mPD1-IL2vHC1: mPD-1.VH-h1.HC.D265A.E356K.N399K.dk-
IL2v.T3A.F42A.Y45A.L72G.C125A murine antiPD-1 (mIgG1 D265A HC1-IL-2v)
EVQLQESGPGLVKPSQSLSLTCSVTGYSITSSYRWNWIRKFPGNRLEWMGYINSAGISNYNPSLKRRISITRDTS
KNQFFLQVNSVTTEDAATYYCARSDNMGTTPFTYWGQGTLVTVSSAKTTPPSVYPLAPGSAAQTNSMVTLGCLVK
GYFPEPVTVTWNSGSLSSGVHTFPAVLQSDLYTLSSSVTVPSSTWPSQTVTCNVAHPASSTKVDKKIVPRDCGCK
PCICTVPEVSSVFIFPPKPKDVLTITLTPKVTCVVVAISKDDPEVQFSWFVDDVEVHTAQTKPREEQINSTFRSV
SELPIMHQDWLNGKEFKCRVNSAAFGAPIEKTISKTKGRPKAPQVYTIPPPKKQMAKDKVSLTCMITNFFPEDIT
VEWQWNGQPAENYKNTQPIMKTDGSYFVYSKLNVQKSNWEAGNTFTCSVLHEGLHNHHTEKSLSHSPAPASSSTK
KTQLQLEHLLLDLQMILNGINNYKNPKLTRMLTAKFAMPKKATELKHLQCLEEELKPLEEVLNGAQSKNFHLRPR
DLISNINVIVLELKGSETTFMCEYADETATIVEFLNRWITFAQSIISTLT
SEQ ID NO: 55: mPD1-IL2v HC2: mPD-1.VH-h1.HC.D265A.K409E.K439D.dk murine
antiPD-1 (mIgG1 D265A HC2)
EVQLQESGPGLVKPSQSLSLTCSVTGYSITSSYRWNWIRKFPGNRLEWMGYINSAGISNYNPSLKRRISITRDTS
KNQFFLQVNSVTTEDAATYYCARSDNMGTTPFTYWGQGTLVTVSSAKTTPPSVYPLAPGSAAQTNSMVTLGCLVK
GYFPEPVTVTWNSGSLSSGVHTFPAVLQSDLYTLSSSVTVPSSTWPSQTVTCNVAHPASSTKVDKKIVPRDCGCK
PCICTVPEVSSVFIFPPKPKDVLTITLTPKVTCVVVAISKDDPEVQFSWFVDDVEVHTAQTKPREEQINSTERSV
SELPIMHQDWLNGKEFKCRVNSAAFGAPIEKTISKTKGRPKAPQVYTIPPPKEQMAKDKVSLTCMITNFFPEDIT
VEWQWNGQPAENYKNTQPIMNTDGSYFVYSELNVQKSNWEAGNTFTCSVLHEGLHNHHTEDSLSHSP
SEQ ID NO: 56: mPD1-IL2v LC: mPD-1.VL-hk.LC murine antiPD-1 (mIgG1 Light Chain)
DIVMTQGTLPNPVPSGESVSITCRSSKSLLYSDGKTYLNWYLQRPGQSPQLLIYWMSTRASGVSDRFSGSGSGTD
FTLKISGVEAEDVGIYYCQQGLEFPTFGGGTKLELKRTDAAPTVSIFPPSSEQLTSGGASVVCFLNNFYPKDINV
KWKIDGSERQNGVLNSWTDQDSKDSTYSMSSTLTLTKDEYERHNSYTCEATHKTSTSPIVKSFNRNEC
SEQ ID NO: 57: hPD-1-IL15 (M1) HC1: xhPD-1.VH-
h1.HC.L234A.L235A.G237A.T366S.L368A.Y407V.dk-IL15m1.N1A.D30N.E46G.V49R
anti-human PD-1 (Fc LALA KiH hole-IL-15m1)
QVQLVQSGAEVKKPGSSVKVSCKASGYTFTSYWINWVRQAPGQGLEWMGNIYPGSSITNYAQKFQGRVTITADES
TSTAYMELSSLRSEDTAVYYCARLTTGTFAYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYICNVNHKPSNTKVDKKVEPKSCDKTHT
CPPCPAPEAAGAPSVFLFPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPCREEMTKNQVSLSCAVKGFYPS
DIAVEWESNGQPENNYKTTPPVLDSDGSFFLVSKLTVDKSRWQQGNVFSCSVMHEALHNHYTQKSLSLSPGSGGG
GSGGGGSGGGGAWVNVISDLKKIEDLIQSMHIDATLYTESNVHPSCKVTAMKCFLLGLQRISLESGDASIHDTVE
NLIILANNSLSSNGNVTESGCKECEELEEKNIKEFLQSFVHIVQMFINTS
SEQ ID NO: 58: hPD-1-IL15 (M1) HC2: xhPD-1.VH-h1.HC.L234A.L235A.G237A.T366W.dk
anti-human PD-1 (Fc LALA KiH knob)
QVQLVQSGAEVKKPGSSVKVSCKASGYTFTSYWINWVRQAPGQGLEWMGNIYPGSSITNYAQKFQGRVTITADES
TSTAYMELSSLRSEDTAVYYCARLTTGTFAYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYICNVNHKPSNTKVDKKVEPKSCDKTHT
CPPCPAPEAAGAPSVFLFPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVCTLPPSREEMTKNQVSLWCLVKGFYPS
DIAVEWESNGQPENNYKTTPPVLDSDGSFFLYSKLTVDKSRWQQGNVFSCSVMHEALHNHYTQKSLSLSPGK
SEQ ID NO: 59: hPD-1-IL15 (M1) LC: xhPD-1.VL-hk.LC anti-human PD-1 (Light Chain)
DIQMTQSPSSLSASVGDRVTITCKSSQSLWDSGNQKNFLTWYQQKPGKAPKLLIYWTSYRESGVPSRFSGSGSGT
DFTLTISSLQPEDFATYYCQNDYFYPLTFGGGTKVEIKRTVAAPSVFIFPPSDEQLKSGTASVVCLLNNFYPREA
KVQWKVDNALQSGNSQESVTEQDSKDSTYSLSSTLTLSKADYEKHKVYACEVTHQGLSSPVTKSFNRGEC
SEQ ID NO: 60: hPD-1-IL15 (M2) HC1: xhPD-1.VH-
h1.HC.L234A.L235A.G237A.T366S.L368A.Y407V.dk-IL15m1.N1G.D30N.E46G.V49R.E64Q
anti-human PD-1 (Fc LALA KiH hole-IL-15m2)
QVQLVQSGAEVKKPGSSVKVSCKASGYTFTSYWINWVRQAPGQGLEWMGNIYPGSSITNYAQKFQGRVTITADES
TSTAYMELSSLRSEDTAVYYCARLTTGTFAYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYICNVNHKPSNTKVDKKVEPKSCDKTHT
CPPCPAPEAAGAPSVFLFPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPCREEMTKNQVSLSCAVKGFYPS
DIAVEWESNGQPENNYKTTPPVLDSDGSFFLVSKLTVDKSRWQQGNVFSCSVMHEALHNHYTQKSLSLSPGSGGG
GSGGGGSGGGGGWVNVISDLKKIEDLIQSMHIDATLYTESNVHPSCKVTAMKCFLLGLQRISLESGDASIHDTVQ
NLIILANNSLSSNGNVTESGCKECEELEEKNIKEFLQSFVHIVQMFINTS
SEQ ID NO: 61: hPD-1-IL15 (M2) HC2: xhPD-1.VH-h1.HC.L234A.L235A.G237A.T366W.dk
anti-human PD-1 (Fc LALA KiH knob)
QVQLVQSGAEVKKPGSSVKVSCKASGYTFTSYWINWVRQAPGQGLEWMGNIYPGSSITNYAQKFQGRVTITADES
TSTAYMELSSLRSEDTAVYYCARLTTGTFAYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYICNVNHKPSNTKVDKKVEPKSCDKTHT
CPPCPAPEAAGAPSVFLFPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVCTLPPSREEMTKNQVSLWCLVKGFYPS
DIAVEWESNGQPENNYKTTPPVLDSDGSFFLYSKLTVDKSRWQQGNVFSCSVMHEALHNHYTQKSLSLSPGK
SEQ ID NO: 62: hPD-1-IL15 (M2) LC: xhPD-1.VL-hk.LC anti-human PD-1 (Light Chain)
DIQMTQSPSSLSASVGDRVTITCKSSQSLWDSGNQKNFLTWYQQKPGKAPKLLIYWTSYRESGVPSRFSGSGSGT
DFTLTISSLQPEDFATYYCQNDYFYPLTFGGGTKVEIKRTVAAPSVFIFPPSDEQLKSGTASVVCLLNNFYPREA
KVQWKVDNALQSGNSQESVTEQDSKDSTYSLSSTLTLSKADYEKHKVYACEVTHQGLSSPVTKSFNRGEC
SEQ ID NO: 63: Kadmon HC1: 2-8 S354C/T366W LALAPG improved linker
ITCPPPMSVEHADIWVKSYSLYSRERYICNSGFKRKAGTSSLTECVLNKATNVAHWTTPS
LKCIRDPALVHQRPAPPSGGGGSGGGGSGGGSGGGGSNWVNVISDLKKIEDLIQSMHIDA
TLYTESDVHPSCKVTAMKCFLLELQVISLESGDASIHDTVESLIILANNSLSSNGNVTES
GCKECEELEEKNIKEFLQSFVHIVQMFINTSGGGGSGGGGSGGGGSGGGGSGGGGSGGGG
SEVQLLESGGGLVQPGGSLRLSCAASGFTFSSYWMSWVRQAPGKGLEWVSAISGSGGSTY
YADSVKGRFTISRDNSKNTLYLQMNSLRAEDTAVYYCASSPLQWVDVWGQGTTVTVSSAS
TKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPEAAGGPS
VFLFPPKPKDTLMISRTPEVTCVVVDVSHEDPEVKFNWYVDGVEVHNAKTKPREEQYNST
YRVVSVLTVLHQDWLNGKEYKCKVSNKALGAPIEKTISKAKGQPREPQVYTLPPCRDELT
KNQVSLWCLVKGFYPSDIAVEWESNGQPENNYKTTPPVLDSDGSFFLYSKLTVDKSRWQQ
GNVFSCSVMHEALHNHYTQKSLSLSPGK
SEQ ID NO: 64: Kadmon HC2: SEQ ID 223: 1-4 Y349C/T366S/L368A/Y407V LALAPG
EVQLLESGGGLVQPGGSLRLSCAASGFTFSSYWMSWVRQAPGKGLEWVSAISGSGGSTYY
ADSVKGRFTISRDNSKNTLYLQMNSLRAEDTAVYYCASSPLQWVDVWGQGTTVTVSSAST
KGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPEAAGGPSV
FLFPPKPKDTLMISRTPEVTCVVVDVSHEDPEVKFNWYVDGVEVHNAKTKPREEQYNSTY
RVVSVLTVLHQDWLNGKEYKCKVSNKALGAPIEKTISKAKGQPREPQVCTLPPSRDELTK
NQVSLSCAVKGFYPSDIAVEWESNGQPENNYKTTPPVLDSDGSFFLVSKLTVDKSRWQQG
NVFSCSVMHEALHNHYTQKSLSLSPGK
SEQ ID NO: 65: Kadmon LC: SEQ ID NO: 219-38B2:
DIQMTQSPSSLSASVGDRVTITCRASESISSWLAWYQQKPGKAPKLLIYDASSLESGVPS
RFSGSGSGTDFTLTISSLQPEDFATYYCQQGDSFPFTFGQGTKLEIKRTVAAPSVFIFPP
SDEQLKSGTASVVCLLNNFYPREAKVQWKVDNALQSGNSQESVTEQDSKDSTYSLSSTLT
LSKADYEKHKVYACEVTHQGLSSPVTKSFNRGEC
SEQ ID NO: 66: mSOT202 HC knob
QVQLVQSGAEVKKPGASVKVSCKASGYTFTDYAMHWVRQAPGQRLEWMGWINTYTGKPTYAQKFQGRVTITRDTS
ASTAYMELSSLRSEDTAVYYCARAVFYGYTMDAWGQGTLVTVSSAKTTAPSVYPLAPVCGDTTGSSVTLGCLVKG
YFPEPVTLTWNSGSLSSGVHTFPAVLQSDLYTLSSSVTVTSSTWPSQSITCNVAHPASSTKVDKKIEPRGPTIKP
CPPCKCPAPNLLGGPSVFIFPPKIKDVLMISLSPIVTCVVVDVSEDDPDVQISWFVNNVEVHTAQTQTHREDYNS
TLRVVSALPIQHQDWMSGKEFKCKVNNKDLPAPIERTISKPKGSVRAPQVYVLPPPEEEMTKKQVTLWCMVTDEM
PEDIYVEWTNNGKTELNYKNTEPVLDSDGSYFMYSKLRVEKKNWVERNSYSCSVVHEGLHNHHTTKSFSRTPGIT
CPPPMSVEHADIWVKSYSLYSRERYICNSGFKRKAGTSSLTECVLNKATNVAHWTTPSLKCIRDPALVHQRPAPP
SGGSGGGGSGGGSGGGGSGGNWVNVISDLKKIEDLIQSMHIDATLYTESDVHPSCKVTAMKCFLLELQVISLESG
DASIHDTVEALIILANNSLSSNAQVTESGCKECEELEEKNIKEFLQSFVHIVQMFINTS
SEQ ID NO: 67: mSOT202 HC hole
QVQLVQSGAEVKKPGASVKVSCKASGYTFTDYAMHWVRQAPGQRLEWMGWINTYTGKPTYAQKFQGRVTITRDTS
ASTAYMELSSLRSEDTAVYYCARAVFYGYTMDAWGQGTLVTVSSAKTTAPSVYPLAPVCGDTTGSSVTLGCLVKG
YFPEPVTLTWNSGSLSSGVHTFPAVLQSDLYTLSSSVTVTSSTWPSQSITCNVAHPASSTKVDKKIEPRGPTIKP
CPPCKCPAPNLLGGPSVFIFPPKIKDVLMISLSPIVTCVVVDVSEDDPDVQISWFVNNVEVHTAQTQTHREDYNS
TLRVVSALPIQHQDWMSGKEFKCKVNNKDLPAPIERTISKPKGSVRAPQVYVLPPPEEEMTKKQVTLSCAVTDFM
PEDIYVEWTNNGKTELNYKNTEPVLDSDGSYFMVSKLRVEKKNWVERNSYSCSVVHEGLHNHHTTKSFSRTPG
SEQ ID NO: 68: mSOT202 LC
DIQMTQSPSSLSASVGDRVTITCRASEDIYSNLAWYQQKPGKAPKLLIFSVKRLQDGVPSRFSGSGSGTDFTLTI
SSLQPEDFATYYCLQGSNFPLTFGQGTKVEIKRADAAPTVSIFPPSSEQLTSGGASVVCFLNNFYPKDINVKWKI
DGSERQNGVLNSWTDQDSKDSTYSMSSTLTLTKDEYERHNSYTCEATHKTSTSPIVKSFNRNEC
SEQ ID NO: 69: mSOT202 LALAPG HC knob
QVQLVQSGAEVKKPGASVKVSCKASGYTFTDYAMHWVRQAPGQRLEWMGWINTYTGKPTYAQKFQGRVTITRDTS
ASTAYMELSSLRSEDTAVYYCARAVFYGYTMDAWGQGTLVTVSSAKTTAPSVYPLAPVCGDTTGSSVTLGCLVKG
YFPEPVTLTWNSGSLSSGVHTFPAVLQSDLYTLSSSVTVTSSTWPSQSITCNVAHPASSTKVDKKIEPRGPTIKP
CPPCKCPAPNAAGGPSVFIFPPKIKDVLMISLSPIVTCVVVDVSEDDPDVQISWFVNNVEVHTAQTQTHREDYNS
TLRVVSALPIQHQDWMSGKEFKCKVNNKDLGAPIERTISKPKGSVRAPQVYVLPPPEEEMTKKQVTLWCMVTDEM
PEDIYVEWTNNGKTELNYKNTEPVLDSDGSYFMYSKLRVEKKNWVERNSYSCSVVHEGLHNHHTTKSFSRTPGIT
CPPPMSVEHADIWVKSYSLYSRERYICNSGFKRKAGTSSLTECVLNKATNVAHWTTPSLKCIRDPALVHQRPAPP
SGGSGGGGSGGGSGGGGSGGNWVNVISDLKKIEDLIQSMHIDATLYTESDVHPSCKVTAMKCFLLELQVISLESG
DASIHDTVEALIILANNSLSSNAQVTESGCKECEELEEKNIKEFLQSFVHIVQMFINTS
SEQ ID NO: 70: mSOT202 LALAPG HC hole
QVQLVQSGAEVKKPGASVKVSCKASGYTFTDYAMHWVRQAPGQRLEWMGWINTYTGKPTYAQKFQGRVTITRDTS
ASTAYMELSSLRSEDTAVYYCARAVFYGYTMDAWGQGTLVTVSSAKTTAPSVYPLAPVCGDTTGSSVTLGCLVKG
YFPEPVTLTWNSGSLSSGVHTFPAVLQSDLYTLSSSVTVTSSTWPSQSITCNVAHPASSTKVDKKIEPRGPTIKP
CPPCKCPAPNAAGGPSVFIFPPKIKDVLMISLSPIVTCVVVDVSEDDPDVQISWFVNNVEVHTAQTQTHREDYNS
TLRVVSALPIQHQDWMSGKEFKCKVNNKDLGAPIERTISKPKGSVRAPQVYVLPPPEEEMTKKQVTLSCAVTDFM
PEDIYVEWTNNGKTELNYKNTEPVLDSDGSYFMVSKLRVEKKNWVERNSYSCSVVHEGLHNHHTTKSFSRTPG
SEQ ID NO: 71: mSOT202 isotype HC knob
EVQLVESGGGLVKPGGSLKLSCAVSGFTFSDYAMSWIRQTPENRLEWVASINIGATYAYYPDSVKGRFTISRDNA
KNTLFLQMSSLGSEDTAMYYCARPGSPYEYDKAYYSMAYWGPGTSVTVSSAKTTAPSVYPLAPVCGDTTGSSVTL
GCLVKGYFPEPVTLTWNSGSLSSGVHTFPAVLQSDLYTLSSSVTVTSSTWPSQSITCNVAHPASSTKVDKKIEPR
GPTIKPCPPCKCPAPNLLGGPSVFIFPPKIKDVLMISLSPIVTCVVVDVSEDDPDVQISWFVNNVEVHTAQTQTH
REDYNSTLRVVSALPIQHQDWMSGKEFKCKVNNKDLPAPIERTISKPKGSVRAPQVYVLPPPEKEMTKKQVTLTC
MVTDEMPEDIYVEWTNNGKTELNYKNTEPVLKSDGSYFMYSKLRVEKKNWVERNSYSCSVVHEGLHNHHTTKSFS
RTPGITCPPPMSVEHADIWVKSYSLYSRERYICNSGFKRKAGTSSLTECVLNKATNVAHWTTPSLKCIRDPALVH
QRPAPPSGGSGGGGSGGGSGGGGSGGNWVNVISDLKKIEDLIQSMHIDATLYTESDVHPSCKVTAMKCFLLELQV
ISLESGDASIHDTVEALIILANNSLSSNAQVTESGCKECEELEEKNIKEFLQSFVHIVQMFINTS
SEQ ID NO: 72: mSOT202 isotype HC hole
EVQLVESGGGLVKPGGSLKLSCAVSGFTFSDYAMSWIRQTPENRLEWVASINIGATYAYYPDSVKGRFTISRDNA
KNTLFLQMSSLGSEDTAMYYCARPGSPYEYDKAYYSMAYWGPGTSVTVSSAKTTAPSVYPLAPVCGDTTGSSVTL
GCLVKGYFPEPVTLTWNSGSLSSGVHTFPAVLQSDLYTLSSSVTVTSSTWPSQSITCNVAHPASSTKVDKKIEPR
GPTIKPCPPCKCPAPNLLGGPSVFIFPPKIKDVLMISLSPIVTCVVVDVSEDDPDVQISWFVNNVEVHTAQTQTH
REDYNSTLRVVSALPIQHQDWMSGKEFKCKVNNKDLPAPIERTISKPKGSVRAPQVYVLPPPEEEMTKKQVTLTC
MVTDFMPEDIYVEWTNNGKTELNYKNTEPVLDSDGSYFMYSELRVEKKNWVERNSYSCSVVHEGLHNHHTTDSES
RTPG
SEQ ID NO: 73: mSOT202 isotype LC
DVQMTQSTSSLSASLGDRVTISCRASQDIKNYLNWYQQKPGGTVKLLIYYSSTLLSGVPSRFSGRGSGTDFSLTI
TNLEREDIATYFCQQSITLPPTFGGGTKLEIKRADAAPTVSIFPPSSEQLTSGGASVVCFLNNFYPKDINVKWKI
DGSERQNGVLNSWTDQDSKDSTYSMSSTLTLTKDEYERHNSYTCEATHKTSTSPIVKSFNRNEC
SEQ ID NO: 74: mSOT202 isotype LALAPG HC knob
EVQLVESGGGLVKPGGSLKLSCAVSGFTFSDYAMSWIRQTPENRLEWVASINIGATYAYYPDSVKGRFTISRDNA
KNTLFLQMSSLGSEDTAMYYCARPGSPYEYDKAYYSMAYWGPGTSVTVSSAKTTAPSVYPLAPVCGDTTGSSVTL
GCLVKGYFPEPVTLTWNSGSLSSGVHTFPAVLQSDLYTLSSSVTVTSSTWPSQSITCNVAHPASSTKVDKKIEPR
GPTIKPCPPCKCPAPNAAGGPSVFIFPPKIKDVLMISLSPIVTCVVVDVSEDDPDVQISWFVNNVEVHTAQTQTH
REDYNSTLRVVSALPIQHQDWMSGKEFKCKVNNKDLGAPIERTISKPKGSVRAPQVYVLPPPEKEMTKKQVTLTC
MVTDFMPEDIYVEWTNNGKTELNYKNTEPVLKSDGSYFMYSKLRVEKKNWVERNSYSCSVVHEGLHNHHTTKSES
RTPGITCPPPMSVEHADIWVKSYSLYSRERYICNSGFKRKAGTSSLTECVLNKATNVAHWTTPSLKCIRDPALVH
QRPAPPSGGSGGGGSGGGSGGGGSGGNWVNVISDLKKIEDLIQSMHIDATLYTESDVHPSCKVTAMKCFLLELQV
ISLESGDASIHDTVEALIILANNSLSSNAQVTESGCKECEELEEKNIKEFLQSFVHIVQMFINTS
SEQ ID NO: 75: mSOT202 isotype LALAPG HC hole
EVQLVESGGGLVKPGGSLKLSCAVSGFTFSDYAMSWIRQTPENRLEWVASINIGATYAYYPDSVKGRFTISRDNA
KNTLFLQMSSLGSEDTAMYYCARPGSPYEYDKAYYSMAYWGPGTSVTVSSAKTTAPSVYPLAPVCGDTTGSSVTL
GCLVKGYFPEPVTLTWNSGSLSSGVHTFPAVLQSDLYTLSSSVTVTSSTWPSQSITCNVAHPASSTKVDKKIEPR
GPTIKPCPPCKCPAPNAAGGPSVFIFPPKIKDVLMISLSPIVTCVVVDVSEDDPDVQISWFVNNVEVHTAQTQTH
REDYNSTLRVVSALPIQHQDWMSGKEFKCKVNNKDLGAPIERTISKPKGSVRAPQVYVLPPPEEEMTKKQVTLTC
MVTDFMPEDIYVEWTNNGKTELNYKNTEPVLDSDGSYFMYSELRVEKKNWVERNSYSCSVVHEGLHNHHTTDSES
RTPG
SEQ ID NO: 76: RLI-15_{AQ} peptide
ELQVISLESGDASIHDTVENLIILANNSLSSNAQV
SEQ ID NO: 77: RLI-15_{AQA} peptide
ELQVISLESGDASIHDTVEALIILANNSLSSNAQV
SEQ ID NO: 78: RLI-15 NA peptide
VEALIILANNSLSSNGNVTESGCKECEELEEK
SEQ ID NO: 79: RLI-15_{AQA} peptide
VEALIILANNSLSSNAQVTESGCKECEELEEK

The invention is further described by the following embodiments:

1. An IL-15 variant comprising amino acid substitutions at position G78 and at position N79 of mature human IL-15.

2. An IL-15 variant comprising SEQ ID NO: 3.

3. The IL-15 variant of embodiment 1 or embodiment 2, wherein the IL-15 variant is glycosylated.

4. The IL-15 variant of any one of embodiments 1-3, wherein glycosylation of the IL-15 variant is reduced in comparison to glycosylated mature human IL-15.

5. The IL-15 variant of any one of embodiments 1-4, wherein glycosylation of the IL-15 variant is increased at N71 of the IL-15 variant in comparison to glycosylate mature human IL-15.

6. The IL-15 variant of any one of embodiments 1-5, wherein the IL-15 variant is obtained by expression of a nucleic acid encoding the IL-15 variant in a mammalian cell.

7. The IL-15 variant of embodiment 6, wherein the mammalian cell is a CHO cell.

8. The IL-15 variant of any one of embodiments 1-7, wherein the IL-15 variant exhibits increased homogeneity in comparison to mature human IL-15.

9. The IL-15 variant of any one of embodiments 1-8, wherein the IL-15 variant further comprises an amino acid substitution that reduces the binding to the IL-2/IL-15Rβ and/or to the γ_{c} receptor and/or the IL-15Rα as described herein.

10. The IL-15 variant of any one of embodiments 1-9, wherein the IL15 variant comprises G78A and N79Q.

11. A composition comprising IL-15 variants of any one of embodiments 1-10, wherein less than 30%, preferably less than 25%, of the IL-15 variants in the composition are glycosylated.

12. A composition comprising IL-15 variants of any one of embodiments 1-11, wherein more than 15% and less than 25% of the IL-15 variants in the composition are glycosylated at N71.

13. The composition of embodiment 11 or 12, wherein the composition exhibits increased homogeneity as compared to a composition comprising mature human IL-15.

14. The composition of any of embodiments 11 to 12, wherein the composition exhibits a more homogenous glycosylation pattern as compared to a composition comprising mature human IL-15.

15. A conjugate comprising the IL-15 variant of any one of embodiments 1-10 and the sushi domain of IL-15Rα or a derivative thereof.

16. A fusion protein comprising the IL-15 variant of any one of embodiments 1-10 and the sushi domain of IL-15Rα or a derivative thereof

17. An immunocytokine comprising the IL-15 variant of any one of embodiments 1-10, the conjugate of embodiment 15 or the fusion protein of embodiment 16 and an antibody or a functional variant thereof.

18. The immunocytokine of embodiment 17, wherein the antibody is an antibody as described herein or a functional variant thereof.

19. The immunocytokine of embodiment 17, wherein the antibody is an immunomodulatory antibody or a functional variant thereof, preferably an antibody directed against PD-1, PD-L1 or PD-L2 or a functional variant thereof.

20. A nucleic acid encoding the IL-15 variant of any one of embodiments 1-10, the conjugate of embodiment 15, the fusion protein of embodiment 16 or the immunocytokine of any one of embodiments 17-19.

21. A vector comprising the nucleic acid of embodiment 20.

22. A host cell comprising the nucleic acid of embodiment 20 or the vector of embodiment 21.

23. A method of preparing the IL-15 variant of any one of embodiments 1-10, the conjugate of embodiment 15, the fusion protein of embodiment 16 or the immunocytokine of any one of embodiments 17-19.

24. The IL-15 variant of any one of embodiments 1-10, the conjugate of embodiment 15, the fusion protein of embodiment 16 or the immunocytokine of any one of embodiments 17-19 for use in treatment.

25. The IL-15 variant of any one of embodiments 1-10, the conjugate of embodiment 15, the fusion protein of embodiment 16 or the immunocytokine of any one of embodiments 17-19 for use in the treatment of a neoplastic disease or an infectious disease.

26. A polypeptide comprising SEQ ID NO: 9 or SEQ ID NO: 10.

The invention is further described by the following embodiments:

1. An interleukin-15 (IL-15) variant comprising amino acid substitutions at position G78 and at position N79 of a mature human IL-15.

2. The IL-15 variant of embodiment 1, wherein the IL-15 variant comprises the amino acid substitutions G78A, G78V, G78L or G78I, and N79Q, N79H or N79M, preferably G78A and N79Q.

3. The IL-15 variant of embodiment 1 or embodiment 2, wherein the IL-15 variant has been expressed in a mammalian cell line, preferably the mammalian cell line is selected from CHO cells, HEK293 cells, COS cells, PER.C6 cells, SP20 cells, NSO cells or any cells derived therefrom, more preferably CHO cells.

4. The IL-15 variant of any of embodiments 1 to 3, wherein the amino acid substitutions
(a) reduce deamidation at N77 and glycosylation at N79 of the IL-15 variant compared to mature human IL-15,
(b) result in less than 30% of glycosylated IL-15 variant, preferably less than 25% of glycosylated IL-15 variant, and/or,
(c) increase glycosylation at N71 of the IL-15 variant compared to mature human IL-15.

5. The IL-15 variant of any of embodiments 1 to 4, wherein the amino acid substitutions do not substantially reduce the IL-15 activity of the IL-15 variant on the proliferation induction of Kit225 cells, 32Db cells, human PBMC or in the Promega IL-15-bioassay.

6. The IL-15 variant of any of embodiments 1 to 5, wherein the IL-15 variant does not have a substitution at position N71 and/or at position N77.

7. The IL-15 variant of any of the embodiments 1 to 6, wherein the IL-15 variant comprises at least one further substitution that reduces the binding to the IL-2/IL-15Rβ and/or to the γc receptor and/or the IL-15Rα.

8. The IL-15 variant of embodiment 7, wherein
(a) the site for the further substitution reducing binding to the IL-2/IL-15Rβ and/or to the γc receptor is selected from the list consisting of N1, N4, S7, D8, K10, K11, D30, D61, E64, N65, L69, N72, E92, Q101, Q108, and I111, preferably from the list consisting of D61, N65 and Q101, most preferably N65;
(b) the further substitution reducing binding to the IL-2/IL-15Rβ and/or to the γc receptor is selected the list consisting of N1D, N1A, N1G, N4D, S7Y, S7A, D8A, D8N, K10A, K11A, D30N, D61A, D61N, E64Q, N65D, N65A, N65E, N65R, N65K, L69R, N72R, Q101D, Q101E, Q108D, Q108A, Q108E and Q108R,
   preferably from the list consisting of D8A, D8N, D61A, D61N, N65A, N65D, N72R, Q101D, Q101E and Q108A,
   more preferably selected from the list consisting of D61A, N65A and Q101, most preferably N65A; or
(c) the further substitution reducing binding to the IL-2/IL-15Rβ and/or to the γc receptor is a combined substitution and is selected form the list consisting of D8N/N65A, D61A/N65A and D61A/N65A/Q101D.

9. The IL-15 variant of embodiment 7, wherein
(a) the site for the further substitution reducing binding to the IL-15Rα is selected from the list consisting of L44, L45, E46, L47, V49, I50, S51, E64, L66, I67, I68 and L69,
(b) the further substitution reducing binding to the IL-15Rα is selected from the list consisting of L44D, E46K, E46G, L47D, V49D, V49R, I50D, L66D, L66E, I67D, and I67E, or
(c) the further substitution reducing binding to the IL-15Rα is a combined substitution selected form the list consisting of E46G/V49R, N1A/D30N/E46G/V49R, N1G/D30N/E46G/V49R/E64Q, V49R/E46G/N1A/D30N and V49R/E46G/N1G/E64Q/D30N.

10. A conjugate comprising an IL-15 variant of any of the embodiments 1 to 9.

11. The conjugate of embodiment 10, wherein the conjugate further comprises the sushi domain of an IL-15Rα or a derivative thereof.

12. A fusion protein comprising an IL-15 variant of any of the embodiments 1 to 9.

13. The fusion protein of embodiment 12, where in the fusion protein further comprises the sushi domain of an IL-15Rα or a derivative thereof, a targeting moiety, and/or a half-life extending moiety, and optionally one or more linker(s).

14. The fusion protein of embodiment 13, wherein the fusion protein comprises, preferably in N- to C-terminal order, the human IL-15Rα sushi domain, a linker and the IL-15 variant of any of the embodiments 1 to 9, preferably wherein the human IL-15Rα sushi domain comprises the sequence of SEQ ID NO: 5,
the linker has a length of 18 to 22 amino acids and is composed of serines and glycines, and more preferably wherein the fusion protein is SEQ ID NO: 9 or SEQ ID NO: 10.

15. The fusion protein of any of the embodiments 12 to 14, wherein the targeting moiety is an antibody or functional variant thereof that preferably binds to a tumor antigen, a tumor extracellular matrix antigen, or a tumor neovascularization antigen, or is an immunomodulatory antibody.

16. The fusion protein of embodiment 15, wherein the fusion protein is fused to the C-terminus of at least one heavy chain of the antibody or to the C-terminus of both light chains of the antibody.

17. A nucleic acid encoding the IL-15 variant of any of the embodiments 1 to 9, the conjugate of any of embodiments 10 or 11, or the fusion protein of any of the embodiments 12 to 16.

18. A vector comprising the nucleic acid of embodiment 17.

19. A host cell comprising the nucleic acid of embodiment 17 or the vector of embodiment 18.

20. The IL-15 variant of any of the embodiments 1 to 9, the conjugate of any of embodiments 10 or 11, or the fusion protein of any of the embodiments 12 to 15, the nucleic acid of embodiment 17 or the vector of embodiment 18 for use in treatment.

21. A pharmaceutical composition comprising the IL-15 variant of any of the embodiments 1 to 9, the conjugate of any of embodiments 10 or 11, or the fusion protein of any of the embodiments 12 to 15, the nucleic acid of embodiment 17 or the vector of embodiment 18 and a pharmaceutically acceptable carrier.

22. The IL-15 variant of any of the embodiments 1 to 9, the conjugate of any of embodiments 10 or 11, or the fusion protein of any of the embodiments 12 to 15, the nucleic acid of embodiment 17 or the vector of embodiment 18 for use in the treatment of a subject suffering from, at risk of developing and/or being diagnosed for a neoplastic disease or an infectious disease.

### EXAMPLES

### 1. Expression and Purification, General Materials and Methods

RLI2 (RLI2 wt), RLI2 with G78A substitution (RLI2 A) and RLI2 with G78A/N79Q substitutions (RLI2 AQ) were expressed transiently in CHO cells and purified from supernatants by supernatant thawing, concentration and diafiltration, optional clarification, Q-sepharose chromatography step, phenyl-sepharose chromatography step, buffer exchange (dialysis) and concentration. In detail:

### Concentration and Diafiltration by TFF1

After thawing, sterile filtrated CHO supernatants (875 mL for RLI2 wt or approximately 2800 mL for mutants) were concentrated and diafiltrated for buffer exchange. CHO supernatants were concentrated from a 2.5-fold factor (for RLI wt) or approximately 5.5 times (for RLI mutants) and diafiltration for buffer exchange (with buffer 25 mM Tris-HCl pH7.5) was performed, with approximately 7 volumes of diafiltration buffer. If necessary, this material was then clarified by centrifugation at 15000 g for 30 minutes at 20° C. and then filtrated on a 0.45 μm PES membrane filter and a 0.22 μm PES membrane filter and immediately injected on Q-sepharose resin.

### Capture by Anion Exchange Chromatography on Q-Sepharose Resin (AEX)

The respective diafiltrated CHO supernatant was loaded at 200 cm/h (50.7 mL/min; residence time 3 min) on a 150 mL-column of Q-sepharose (diameter 44 mm, bed height 10 cm) after prior equilibration in buffer B (25 mM Tris HCl pH 7.5, 1 M NaCl) then buffer A (25 mM Tris HCl pH 7.5). After loading, the column was washed with 10 CV of buffer A at the same flow rate. The protein was eluted from the column with increasing salt concentration: a first 15 CV linear gradient was applied from 0% to 25% buffer B (25 mM Tris HCl pH 7.5, 1 M NaCl), followed by a 5 CV step at 25% buffer B (step 1) and a 10 CV step at 100% buffer B (step 2). Finally, a 10 CV re-equilibration step was applied with buffer A. Purification was followed with UV signal at 280 nm.

Elution in linear gradient was fractionated and collected in 40-mL fractions for the 10 first CV then 5 CV were collected in F5 fraction. Step at 250 mM NaCl, at 1 M NaCl and re-equilibration were collected in F6, F7 and F8 fractions, respectively. Purification fractions were analyzed by SDS-PAGE and anti-RLI Western blot for determination of elution pool.

### Purification by Hydrophobic Chromatography on Phenyl-Sepharose Resin

The respective Q-sepharose elution pool was loaded at 149 cm/h (20 mL/min; residence time 5 min), with a 1.6-fold online dilution in buffer B (25 mM Tris-HCl pH 7.5; 2 M ammonium sulfate) up to 750 mM ammonium sulfate, on a 100 mL phenyl-sepharose column (diameter 32 mm, bed height 12.4 cm) after prior equilibration in a mix of 62.5% buffer A (25 mM Tris HCl pH 7.5) and 37.5% buffer B (25 mM Tris-HCl pH 7.5; 2 M ammonium sulfate). After loading, the column was washed with 5 CV of mix 62.5% buffer A/37.5% buffer B at the same flow rate. The protein was eluted from the column with decreasing salt concentration: a 20 CV linear gradient was applied from 37.5% to 0% buffer B, followed by a 5 CV step at 100% A (step 2). Finally, a 5 CV step was applied with buffer C (isopropanol 30%, step 3) for stripping. Purification was followed with UV signal at 280 nm. Elution in linear gradient was fractionated and collected in 40-mL fractions. Purification fractions were analyzed by SDS-PAGE and anti-RLI Western blot for determination of elution pool.

### Formulation Step: Concentration and Diafiltration by TFF

Phenyl-sepharose elution pools were concentrated from a 2.6 to 4.4-fold factor and diafiltration for buffer exchange (with formulation buffer 20 mM L-histidine, 6% D-sorbitol, pH 6.5) was performed, with at least 7 volumes of diafiltration buffer. This material was then immediately concentrated on Vivaspin unit with 10 kDa cut-off to reach the final target concentration.

### Concentration

Diafiltrated samples were concentrated using Vivaspin unit with 10 kDa cut-off Concentration was carried out up to reach the theoretical concentration of 1 mg/mL.

### Potency Assay on Kit225

The activity of both IL-2 and IL-15 can be determined by induction of proliferation of kit225 cells as described by Hori et al. (1987). Kit225 cells (Hori, Uchiyama et al. 1987) were passaged in kit225 base medium and used for the potency assay at passage 4-7. Before the potency assay, kit225 cell were cultivated in kit225 base medium without IL-2 for 24 h (starvation period). 1×10⁴ kit225 cells were plated in 96-well plate and a serial dilution of RLI-15 and respective molecules PEM-RLI-15 was added to cells. Cells were incubated at 37° C., 5% CO₂ for 72±3 h. Following the incubation, 10 μl (10% of the volume in the well) of Alamar Blue was added to each well and, after 6 h, absorbance was measured at 560 nm with a 620 nm reference using a Tecan Spark absorbance microplate reader (set mixing before detection for 15 s). In some cases, when lower potency RLI2 mutants were tested, the incubation with kit225 cells was prolonged from 3 days (72 h±3 h) to 5 days.

Preferably, methods such as colorimetry or fluorescence are used to determine proliferation activation due to IL-2 or IL-15 stimulation, as for example described by Soman et al. using CTLL-2 cells (Soman, Yang et al. 2009). As an alternative to cell lines such as the kit225 cells, human peripheral blood mononuclear cells (PBMCs) or buffy coats can be used. A preferred bioassay to determine the activity of IL-2 or IL-15 is the IL-2/IL-15 Bioassay Kit using STAT5-RE CTLL-2 cells (Promega Catalog number CS2018B03/B07/B05).

Concentration of Analyzed RLI Variants were:

| | |
|---|---|
| RLI2 supernatant. | 0.133 mg/ml (ELISA, average from 2 exps) |
| RLI2_{AQ} supernatant. | 0.0297 mg/ml (ELISA, average from 2 exps) |

### Properties of RLI2

| | |
|---|---|
| Purity (RP-UPLC) | 99.8% |
| Formulation | 20 mM histidine, 6% (w/v) sorbitol, pH 6.5 |
| Storage temperature | −20° C. |

### Kit225 Base Medium

RPMI (460 mL)+FBS (30 mL)+Glutamax (5 mL)+Penicillin-Streptomycin (5 mL)+cytokines added into the flasks (75 cm2); IL-2 (5 ng/mL). Cytokines were added to the medium just before cultivation.

hPBMC Potency Assay

Buffy coats were obtained from healthy donors. PBMC were isolated by Ficoll Paque gradient, washed three times and resuspended in T cell complete medium in 96-well plate. Immunocytokines were added at the indicated concentrations and plates were incubated in 37° C. with 5% CO₂ for 7 days. The proliferation of immune cell population was detected by flow cytometry.

### T Cell Complete Medium

RPMI 1640 medium, CTS GutaMAX-I 1×, 100 U/mL Penicillin-Streptomycin, 1 mM Sodium pyruvate, NEAA 1× (non-essential amino acid mix), 2-Mercaptoethanol 0.05 mM and 10% AB human serum (heat inactivated).

### List of Used Antibodies

| Marker | Fluorochrome | Vendor | Cat. No. | Clone |
|---|---|---|---|---|
| CD16 | PE-Cy7 | BioLegend | 302016 | 3G8 |
| CD25 | BV605 | BD | 562660 | 2A3 |
| CD3 | APC ef780 | Thermo | 47-0037-42 | OKT3 |
| CD4 | BV421 | BioLegend | 317434 | OKT4 |
| CD45 | BV605 | BD | 564047 | HI30 |
| CD45 | PerCP-Cy5.5 | BD | 564105 | HI30 |
| CD45RA | PE-Cy7 | BioLegend | 304126 | HI100 |
| CD45RO | A700 | BioLegend | 304218 | UCHL1 |
| CD56 | A700 | BioLegend | 318316 | HCD56 |
| CD8 | PerCP-Cy5.5 | BioLegend | 344710 | SK1 |
| Foxp3 | A488 | Thermo | 53-4776-42 | PCH101 |
| Ki67 | PE | BioLegend | 350504 | ki-67 |
| NKG2D | APC | BioLegend | 320808 | 1D11 |
| PD-1 | FITC | BioLegend | 329904 | EH12 |
| Zombie Aqua | BV510 | BioLegend | 423102 | NA |

Isolation of human NK cells (hNK): Fresh blood from healthy donors was diluted in a 1:1 ration with cold PBS-EDTA, ph7.4 and PBMC were isolated by Ficoll-Paque gradient isolation. Isolated PBMCs were resuspended in complete culture medium. hNK cells were isolated from a the PBMC using the EasySep Human NK Cell Isolation kit (Stem Cell Technologies, USA) according to the manufacturer instructions. Isolate hNK cells of each donor were resuspended in NK medium with 10% serum at a concentration of 3×10⁶ cells/ml.

### PD-1/PD-L1 Blockade Bioassay

The assay was performed according to manufacturer's instructions (Promega PD-1/PD-L1 Blockade Bioassay 11250). In brief, PD-L1 aAPC/CHO-K1 cells were plated in 96 well plate and incubated 16-20 hours in a 37° C., 500 CO₂ incubator. After that PEM-RLI immunocytokines at the indicated concentrations and PD-1 Effector Cells were added to the cells and incubated for 6 hours in a 37 C, 50 CO₂ incubator. After the incubation perod, Bio-Glo™ Reagent was added to the wells and incubated at room temperature for 15 mi, luminescence measurement was performed.

### Cynomolgus Monkey Studies

Pharmacokinetics of indicated PEM-RLI molecules were tested in cynomolgus monkeys (n=2-3) after administration of indicated doses on day 1 or day 15. Blood for serum separation was collected at 1 h, 4 h, 8 h, 24 h, 48 h, 60 h, 72 h, 84 h, 96 h, 120 h and 168 h after administration (some timepoints may have been omitted in some cases). The concentration of immunocytokines in serum was determined by ELISA using the antibodies of Table 3. Blood for flow cytometry evaluation of selected immune cell populations (NK and CD8⁺ T cells) was collected at pre-dose, day 5, 8, 12, 15, 19, 22 and 26.

TABLE 3
List of used antibodies for the cynomolgus monkey studies

| Marker | Fluorochrome | Vendor | Cat. No. | Clone |
|---|---|---|---|---|
| CD45 | PE-Cy7 | BD biosciences | 561294 | D058-1283 |
| CD3 | APC-Cy7 or | BD biosciences | 557757 | SP34-2 |
| | PerCP | BD biosciences | 552851 | |
| CD4 | V450 | BD biosciences | 560811 | L200 |
| CD8 | HV605 or | BD biosciences | 564116 | SK1 |
| | V500 | | 561618 | |
| CD28 | APC | BD biosciences | 560683 | CD28.2 |
| CD95 | FITC | BD biosciences | 555673 | DX2 |
| CD122 | PE | BD biosciences | 557323 | Mik-β2 |
| Fix. | eFluor ™506 or | eBioscience | 65-0866-14 | — |
| Viab. | eFluor ™ 780 | | 65-0865-14 | |
| Dye | | | | |
| Ki-67 | AF700 or | BD biosciences | 561277 | B56 |
| | A488 | | 558616 | |
| CD20 | PE or | BD biosciences | 555623 | 2H7 |
| | APC | BioLegend | 302310 | |
| CD25 | APC | eBioscience | 17-0257-42 | CD25-4E3 |
| Foxp3 | AF488 or | Biolegend | 320112 | 206D |
| | PE | | 320108 | |

### List of Antibodies Used for Error! Reference Source not Found. (Tscm Cell Panel)

| Marker | Fluorochrome | Vendor | Clone |
|---|---|---|---|
| CD3 | PE-Cy7 | eBioscience | 145-2C11 |
| CD8 | V500 | BD Biosciences | 53-6.7 |
| CD44 | APC | eBioscience | IM7 |
| CD122 | BV605 | BD Biosciences | 5H4 |
| CD62L | PE | eBioscience | W18021D |
| CD95 | FITC | eBioscience | 15A7 |
| CXCR3 | PE/Dazzle 594 | BioLegend | CXCR3-173 |
| CCR7 | PerCP/Cy5.5 | BioLegend | 4B12 |
| gdTCR | eF450 | eBioscience | eBioGL3 |
| Sca1 | A700 | BioLegend | D7 |
| FVD | eF780 | eBioscience | — |

### Mouse Efficacy Studies

The objective of these studies was to evaluate the in vivo therapeutic efficacy of PEM-RLI2 NA x1 and Pembrolizumab as a monotherapy in the treatment of HuCell MC38-hPD-L1 tumour cell line in female hPD1 single KI HuGEMM mice (C57BL/6-Pdcd1^{em1(hPDCD1)/Smoc}) (n=8 mice per group). Each mouse was inoculated subcutaneously in the right lower flank region with MC38-hPD-L1 tumour cells (1×10⁶) in 0.1 ml of PBS for tumour development. The randomization was started when the mean tumour size reached 108 mm³. 40 mice were enrolled in the study. All animals were randomly allocated to 5 study groups. Randomization was performed based on “Matched distribution” method (StudyDirector™ software, version 3.1.399.19). The date of randomization day was denoted as day 0 (D0). After tumour cells inoculation, the animals were checked daily (or more often as needed, at the discretion of the Study Director) for morbidity and mortality. Tumour volumes were measured three times per week in two dimensions using a calliper, and the volume were expressed in mm³ using the formula: “V=(L×W×W)/2, where V is tumour volume, L is tumour length (the longest tumour dimension) and W is tumour width (the longest tumour dimension perpendicular to L). PEM-RLI2 NA x1 was administered IV at 20 mg/kg at day 0 and pembrolizumab was administered IP at 5 mg/kg at days 0, 3, 6 and 9. Tumour observation was followed for 18 days. Concomitantly to this, PEM-RLI2 NA x1 (IL-15 with N65A and AQ mutation) was administered IV at 5, 10 at day 0. Tumour observation was followed for 6 days.

### Mixed Lymphocyte Reaction

Buffy coats were obtained from healthy donors. PBMC were isolated by Ficoll Paque gradient, washed three times. PBMC were isolated by Ficoll Paque gradient, washed three times. Pairs of hPBMCs donors were cultivated with equimolar concentration of pembrolizumab and PEM L-RLI NA x1 at 1 nM for six days. IFNγ production in cell supernatants was determined using human IFN-γ DuoSet ELISA (R&D systems, No. DY258B). Data are expressed as relative response of IFNγ production [%] and represent mean±SEM from—12 pairs of hPBMC healthy donors.

### 2. SDS-PAGE and Anti-RLI Western-Blot Analysis

The purified proteins from example 1 were analyzed by SDS-PAGE and anti-RLI Western blot.

Coomassie staining: protein bands are visualized according to their molecular weight in denatured conditions.

Briefly, 1 volume of loading buffer (containing or not beta mercaptoethanol) was added to 3 volumes of the sample to analyze (then more or less diluted into 1× loading buffer), homogenized and denatured 5 min at 95° C. Denatured sample is loaded on Criterion TGX gel and run in running buffer at constant voltage (300 V) and limited current (75 mA or 135 mA per gel depending on the gel type) in 1×TGS buffer for 18 min or 21 min depending on the gel type. Gel is removed from the cassette and washed 3 times 5 min in water, stained 20 min with Biosafe staining solution (Biorad) and washed 3 times 20 min in water before final de-stain wash 3 hours in water. Stained gel is then scanned with gel scanner.

Western-blot analysis: the gel is then transferred to a nitrocellulose membrane and used for Western-blot analysis with different antibodies. At the end of migration, the gel is used for protein transfer to nitrocellulose membrane. For the example of reference (Biorad #170-4155, Trans-Blot® Turbo™ Transfer Starter System), the transfer parameters are 2.5 A, 25 V, 7 minutes (for Criterion gels) or 2.5 A, 25 V, 3 minutes (for Mini-PROTEAN gels). After membrane saturation in iBind™ Flex solution, antibody incubation and wash steps are then done in iBind system. After revelation and when completely dry, the membrane is scanned for analysis. Primary antibody used was anti RLI2-PR01 antibody (Cytune, dilution 1:25000), secondary antibody used was donkey anti-Rabbit IgG-AP antibody (Santa Cruz Biotechnology, dilution 1:5000).

### 3. Capillary Electrophoresis

Protein analysis by capillary electrophoresis relies on separation of LDS-labeled protein variants by a sieving matrix in a constant electric field. The Labchip GXII instrument uses a single sipper icrofluidic chip to characterize protein samples loaded on a 96-well plate. The microfluidic chip technology allows the separation and analysis of the protein samples. After laser-induced signal detection and analysis, the provided data are: relative protein concentration, molecular size and percent purity using ladder and marker calibration standards.

Samples are denatured by mixing 5 μL-sample and 35 μL of HT Protein Sample Buffer in presence or not of DTT at final concentration of 35 mM. If required, samples are prediluted at 1 mg/mL in HT Protein Sample Buffer. Denaturation is performed by heating mix at 100° C. for 5 min. Then, 70 μL of water are added and samples are centrifuged 10 minutes at 2,000 g. Samples (in a 96-well plate) are then loaded on LabChip GXII instrument for chip transfer and analysis.

TABLE 4
Summary of characteristics

| | RLI2 wt | RLI2 A | RLI2 AQ |
|---|---|---|---|
| Capillary Electrophoresis (non- | 6 bands between | 5 bands between | 4 bands between |
| reducing): MW, | 23 and 34 kDa | 23 and 34 kDa | 23 and 34 kDa |
| Purity | 86.5% | 82.2% | 95.0% |
| SDS-PAGE / anti-RLI Western | 92.4% | 90.5% | 93.7% |
| blot (non-reducing): Purity | | | |

### 4. Glycosylation/Deamidation Mutants

TABLE 5
Overview of relevant amino acids
for glycosylation and deamidation

| | | | | | 3^{rd} |
|---|---|---|---|---|---|
| | Minor | Deamida- | | Major | potential |
| | glycosylation | tion | | glycosylation | glycosylation |
| | site | site | | site | site |
| mature | N71 | N77 | G78A | N79Q | N160 |
| IL-15 | | | | | |
| RLI | N168 | N174 | G175A | N176Q | N209 |

The RL12 molecule has the major glycosylation site is N176 (RLI numbering) and a minor site at N168. No glycosylation is seen at N209. The glycans are complex, majorly biantennary, fucosylated, G0 to G2 with little sialylation. In cell culture about 40 to 50% of the protein are glycosylated with about 5% at N168. After purification as described above, about 14-25% of RLI2 are glycosylated. Whereas the different levels of glycosylation have not shown any impact on potency, stability and only a minor impact on pharmacokinetics with glycosylated RLI2 having a shorter half-life, heterogeneity of an active pharmacological ingredient is still problematic from a regulatory perspective.

A potential hot spot for deamidation identified in IL-15 expressed in E. coli (Nellis et al. 2012) is N77 (IL-15 numbering)/N174 (RLI numbering). Although it has been described that N-glycosylation of N79 partially prevents N77 deamidation (Thaysen-Andersen et al. 2016), the inventors indeed saw in mass spectrometry that N77 was deamidated in CHO-expressed RLI2 and identified deamidation as a real problem for potential heterogeneity of RLI2 and RLI-based products and therefore deamidation should be avoided.

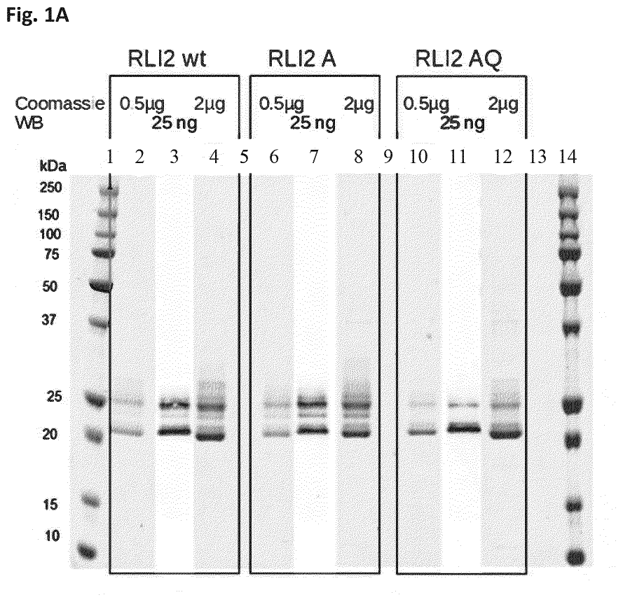

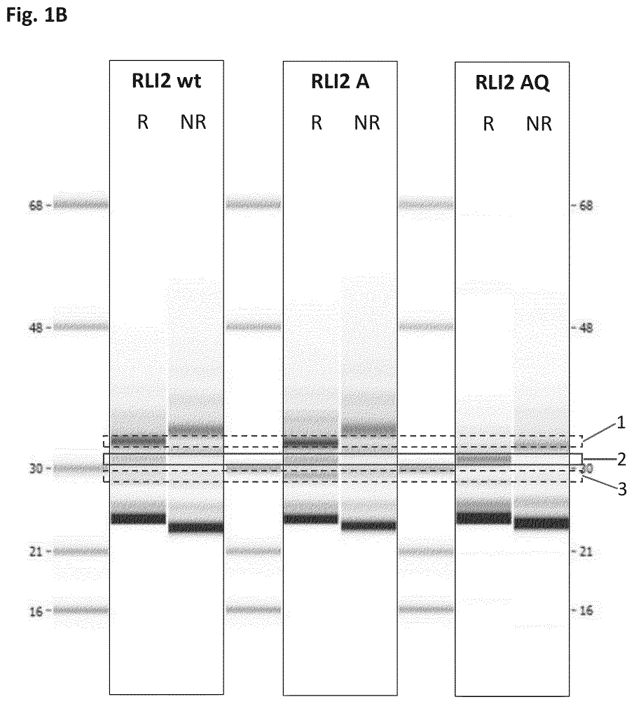

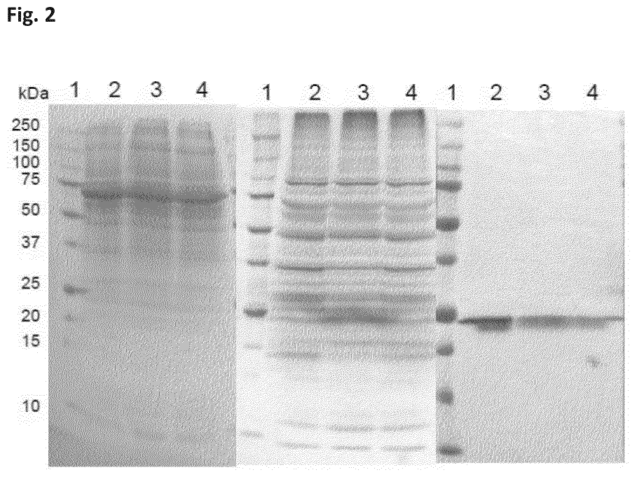

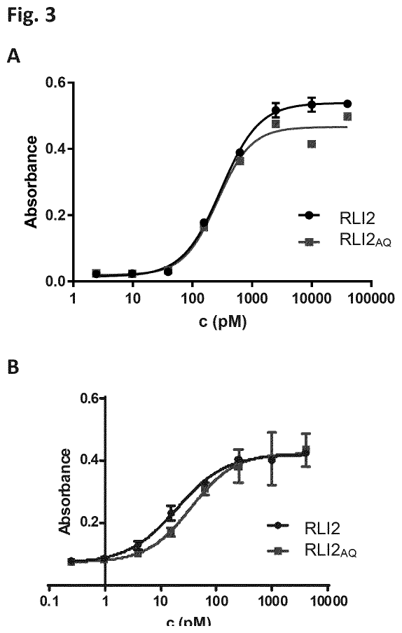

FIG. 1A shows that RLI2 wt (without a mutation) indeed is a heterogenous product with two major bands at about 20 and 25 kDa and a few minor bands, all being immune reactive to the anti-RLI2 antibody and thereby being different modifications of the RLI2 protein.

The inventors wanted to avoid mutating N77 as an obvious way to abolish deamidation of it and thereby removing the polar amide, as the conservative substitution to glutamine would not have resolved the deamidation risk. The single substitution G78A (IL-15 numbering)/G175A (RLI numbering) in RLI2 (RLI2 A) was introduced instead to abolish potential deamidation at position N77. Whereas loss of deamidation would not be visible on the Coomassie staining or the Western blot, the major acidic peak (pI 6.0) in RP-UPLC was significantly reduced in cIEF as it would be expected for loss of deamidation, which confirms that deamidation hot spot N174 indeed was deamidated (data not shown). Also, mass spectrometry analysis of the PEM-RLI AQ constructs showed zero deamidation (data not shown).

Surprisingly the G78A mutation lead to a slight increase in glycosylation (see FIG. 1A and better visible in FIG. 1B) with more larger/more glycosylated species compared to RLI2 wt. An additional band appears indicating such new glycosylation pattern (see dashed box 3 in FIG. 1B). Also RP-UPLC peaks were slightly shifted (data not shown). This changed glycosylation pattern was unexpected as the influence of the deamination mutation G78A on glycosylation could not have been predicted.

By the additionally substituting N79 (IL-15 numbering)/N176 (RLI numbering) by Q (RLI2 AQ, RLI2_{AQ}), which was introduced to disrupt the main glycosylation site of IL-15, a marked reduction of larger species of RLI2 was observed (see dashed box 1 in FIG. 1B). The residual larger band (see solid box 2 in FIG. 1B) likely represents glycosylation at N71 (IL-15 numbering)/N168 (RLI numbering) of about 20% of the RLI molecule, which appears to be slightly increased compared to RLI2 wt and RLI2. The band of box 1 may represent RLI2 glycosylated at N176, whereas the band of box 3 may represent RLI2 glycosylated at N176 and N168. The band of box 3 may however also be RLI2 glycosylated with unfavorable Sialic acid glycan structures at N176. Without being bound by any theory, this surprising increase of glycosylation at N71 may be explained that the glycosylation at the major site N79 sterically hindered glycosylation at N71 in RLI2 wt, such hinderance being relieved once N79 is mutated.

Together, RLI2_{AQ}, and accordingly also IL-15_{AQ}, with the AQ substitutions represent an RLI2, or IL-15, variant with a highly improved homogeneity and a reduced risk for deamidation.

In order to compare the effect/impact of glycosylation on the biological activities of RLI variants, we have specifically inactivated the 3 potential glycosylation sites N71/N79/N160 of IL-15 (N168/N176/N209 for RLI) by site-directed mutagenesis (Stratagene Site Directed Mutagenesis XL Kit). N71 was substituted by S, N79 was substituted by Q and N160 was substituted by S, thereby generating RLI2_{N168S/N176Q/N209S} and RLI1_{N168S/N176Q/N209S}. In order to confirm the main N-glycosylation occupancy on N79 (=N176 of RLI) the RLI2_{N176Q} mutant was made. Transient expression in CHO cells lead to a unique 25 kDa band (see FIG. 2, right pane).

The RLI protein mutated only on its major glycosylation site (RLI2_{N176Q}) exhibited also a unique 25 kDa band, therefore confirming the main glycosylation occupancy on the N176 residue of RLI expressed in CHO (transient expression). Secretion yields of the deglycosylated mutants expressed in in transient CHO cells were similar to their glycosylation/original counterpart. Accordingly, there was no significant influence of the deglycosylation on the expression levels. Same was observed in the Pichia pastoris expression system (data not shown).

Furthermore, these mutations on the N-glycosylation sites appear to induce no significant influence on the in vitro proliferative activity of RLI on kit225 or 32D0 cells. As usually, all RLI versions (RLI1 or RLI2, glycosylated or non-glycosylated, CHO or baculo or Pichia) were similarly stimulating the proliferation of the kit225 cell line.

### 5. Potency of RLI2_{AQ} Variant

The activity of both IL-2 and IL-15 can be determined by induction of proliferation of kit225 cells as described by Hori et al. (1987). Kit225 cells (Hori et al. 1987) were passaged in kit225 base medium and used for the potency assay at passage 4-7. Before the potency assay, kit225 cell were cultivated in kit225 base medium without IL-2 for 24 h (starvation period). 1×10⁴ kit225 cells were plated in 96-well plate and a serial dilution of RLI-15 and respective molecules PEM-RLI-15 was added to cells. Cells were incubated at 37° C., 5% CO₂ for 72±3 h. Following the incubation, 10 μl (10% of the volume in the well) of Alamar Blue was added to each well and, after 6 h, absorbance was measured at 560 nm with a 620 nm reference using a Tecan Spark absorbance microplate reader (set mixing before detection for 15 s). In some cases, when lower potency RLI2 mutants were tested, the incubation with kit225 cells was prolonged from 3 days (72 h±3 h) to 5 days.

Preferably, methods such as colorimetry or fluorescence are used to determine proliferation activation due to IL-2 or IL-15 stimulation, as for example described by Soman et al. using CTLL-2 cells (Soman et al. 2009). As an alternative to cell lines such as the kit225 cells, human peripheral blood mononuclear cells (PBMCs) or buffy coats can be used. A preferred bioassay to determine the activity of IL-2 or IL-15 is the IL-2/IL-15 Bioassay Kit using STAT5-RE CTLL-2 cells (Promega Catalog number CS2018B03/B07/B05).

Concentration of Analyzed RLI Variants were:

| | |
|---|---|
| RLI2 supernatant. | 0.133 mg/ml (ELISA, average from 2 exps) |
| RLI2_{AQ} supernatant. | 0.0297 mg/ml (ELISA, average from 2 exps) |

TABLE 6
EC50 values (nM) of RLI2 compared to RLI2_{AQ} from supernatants
determined by activation of 32Db cells or kit225 cells

| EC50 (nM) | RLI2 | RLI2_{AQ} |
|---|---|---|
| 32Db - plate_21h (FIG. 3A) | 301.1 | 263.1 |
| kit225 - plate_4h (FIG. 3B) | 19.52 | 35.15 |

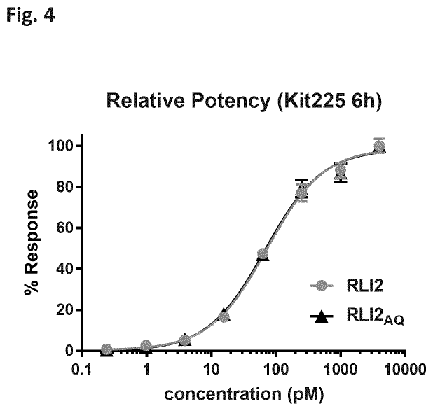

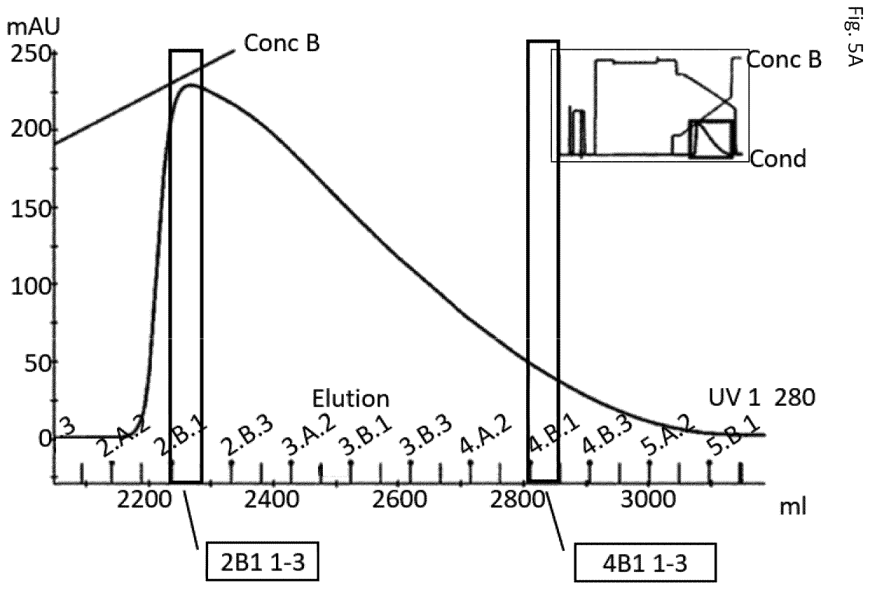

TABLE 7
Relative potency of RLI2 compared to RLI2_{AQ} from supernatants
determined by activation of Kit225 cell proliferation.

| | RLI2 | RLI2_{AQ} |
|---|---|---|
| kit225 - 6h / EC50 (nM) (FIG. 4) | 72.47 | 69.5 |
| Relative potency | 100% | 96% |

Accordingly, the glycosylation mutant RLI2_{AQ} as supernatant showed a very similar potency to stimulate kit225 and/or 32Db cells if compared to RLI2 from supernatant. This was surprising as for many glycoproteins loss of glycosylation leads to a lower activity.

Also in SPR (Biacore) binding experiments to the IL-2/IL-15 βγ receptor, no relevant difference in the kₒₙ rate, k_{off} rate and equilibrium constant K_{d} between RLI2 and RLI AQ was observed (data not shown).

In summary, RLI2_{AQ}, and accordingly also IL-15_{AQ}, with the AQ substitutions represents an RLI2, or IL-15, variant with a highly improved homogeneity, a reduced risk for deamidation with a comparable potency to activate immune cells.

### 6. Cynomolgus PK/PD Study of Highly Glycosylated and Low Glycosylated RLI2

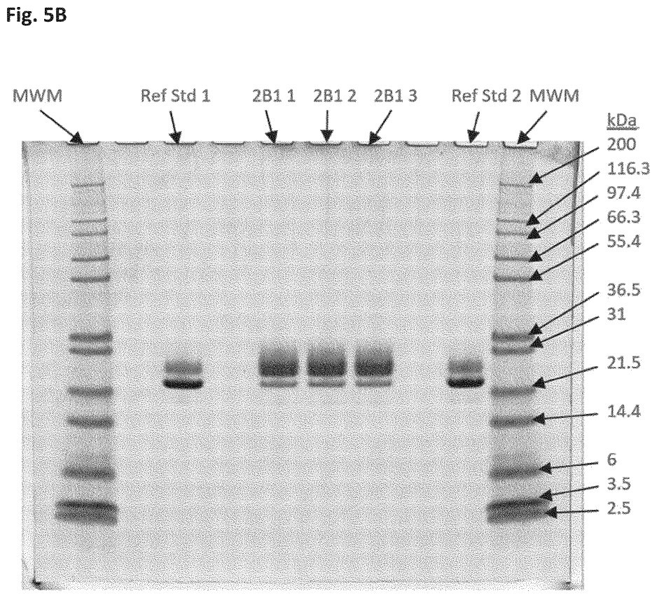

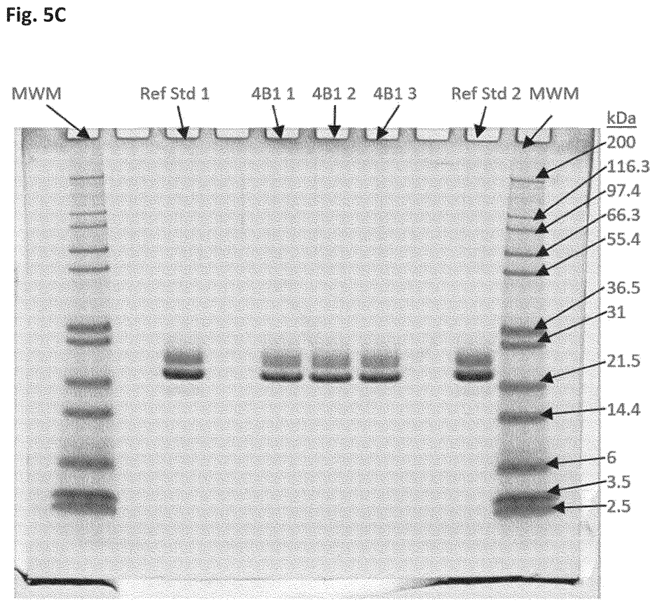

In order to compare highly glycosylated and low glycosylated RLI2 with respect to their PK and PD properties, a 200 l scale production campaign was run, harvested with S0SP and X0SP depth filters and protein was captured on a PPA column. Virus was inactivated by solvent detergent treatment and purification continued via a Capto Adhere column and a Hydroxyapatite type II column (flow through mode), followed by a second virus removal step by Nanofiltration. The RLI preparation was polished on an Capto Impres Phenyl column (CPI Phenyl HIC) and selected fractions for highly glycosylated RL12 were pooled (RLI-15-HG), and selected fractions for low glycosylated RLI2 were pooled (RLI-15-LG), see FIG. 5A-C. Finally, UFDF filtration was performed on a 10 kDa cut-off UF membrane into final formulation buffer (20 mM Histidine, 6% Sorbitol pH6.5). RLI-15-HG shows most of RLI in the upper band for the glycosylated RLI isomer, whereas RLI-15-LG contains only a smaller fraction of glycosylated RLI isomer (FIGS. 5B and C).

A total of three male and three female cynomolgus monkeys were included in PK/PD study. Animals were allocated into two groups receiving RLI2 as RLI-15-HG and RL1-15-LG at 15 μg/'kg (nominal dose) by subcutaneous daily administration according to a cross-over dosing design. Administration was performed for 2 periods of 4 days (2×4), separated by a washout interval of 10 days (Day 1 to Day 4: RLI-15-LG for males and RLI-15-HG for females. Day 15 to Day 18: RLI-15-HG for males and RLI-15-LG for females). Pharmacodynamic parameters (including Ki67 expression in NK, CD4⁺ and CD8⁺ cells) were analyzed from the blood samples collected on pretreatment period. Day 5. Day 12. and Day 19. Blood samples for pharmacokinetic investigations were collected from all animals on Day 1 and Day 15, following the first administration in each treatment interval, at the following time-points: pre-dose. and 0.5, 1, 2, 6, 12 and 24 hours after administration. Bioanalysis was performed. Additionally, backup serum samples (D1 (predose). D15 (predose) and D16 (24 h)) were partially used for immunogenicity assessment (ADA determination).

Pharmacokinetic (PK) analysis was performed using non-compartmental analysis on Phoenix™ WinNonlin® software (version 6.4. Certara L.P.).

Pharmacokinetic profile: All treated animals were exposed to the test item as quantifiable amount of RLI2 were measured over a major part of the sampling period after administration on Day 1 and Day 15. The main pharmacokinetic parameters are summarized in Table 8.

TABLE 8
Main pharmacokinetic parameters

| | | RLI2 LG | | RLI2 HG | |
|---|---|---|---|---|---|
| | | male | female | male | female |
| AUC₀₋ₜ ± SD | ng · h/ml | 43.9 ± 6.7 | 84.3 ± 28.4 | 19.6 ± 3.2 | 37.9 ± 4.4 |
| AUC₀₋ₜ/dose ± SD | ng · h/ml/(mg/kg) | 2.8 ± 0.3 | 5.6 ± 1.9 | 1.3 ± 0.3 | 2.5 ± 0.3 |
| Cₘₐₓ ± SD | ng/ml | 6.1 ± 1.4 | 9.0 ± 2.5 | 3.0 ± 0.7 | 7.6 ± 4.8 |
| Cₘₐₓ/dose ± SD | ng/ml/(mg/kg) | 0.4 ± 0.1 | 0.6 ± 0.2 | 0.2 ± 0.1 | 0.5 ± 0.3 |
| Tₘₐₓ (min-max) | h | 2 (2-2) | 2 (1-6) | 2 (2-2) | 2 (1-2) |

Exposure by means of Cₘₐₓ and AUC₀₋ₜ was different between male and female animals. Cₘₐₓ and AUC₀ₜ was about 2-fold higher in females than in males. Independent of this gender difference. a difference in the pharmacokinetics of RLI-15-HG and RLI-15-LG was also observed. Surprisingly, exposure by RLI-15-HG was lower than exposure by RLI-15-LG. The ratio between RLI-15-HG and RLI-15-LG were 0.606 and 0.453 for Cₘₐₓ and AUC₀₋ₜ respectively, independently on animal sex.

### 7. Generation of RLI2_{AQ}-Based Immunocytokines

Immunocytokines were generated where either two RLI2AQ fusion proteins were fused without a linker to the C-terminus of the heavy chains of an anti-PD-1 antibody/IgG4 or one RLI2AQ fusion protein was fused to one heavy chain (the knob chain) using the know-in-whole technology (KIH) with HC knob mutation T366W and HC hole chain mutations T366S/L368A/Y407V. The ant-PD-1 antibody is pembrolizumab (PEM) with or without Fc mutations as shown in Table 9.

TABLE 9
PEM-RLI2 immunocytokines all having AQ mutation
(G175A/N176Q) with Fc mutations: “L” or “LE”
= L235E (further ADCC reduction), “Y” or “YTE” =
M252Y/S254T/T256E (increased FcRn binding for half-life extension),
LY = combination of L and Y. Further IL-15 mutations: KAQD =
K10A/Q101D DA = D61A, NA = N65A, ND = N65D, NQD = D12
7N/E161Q/N162D (for reducing binding of IL-15/RLI to the
IL-2Rβγ in order to increase half-life of the respective
immunocytokine).

| Construct name | mutations |
|---|---|
| PEM | Control PEM |
| PEM-KIH | Control PEM with KIH mutation |
| PEM-RLI x1 | monomeric RLI2 constructs; |
| PEM-RLI KAQD x1 | with Fc wt of PEM (only KIH) |
| PEM-RLI DA x1 | IL-15 with indicated mutations (bold) |
| PEM-RLI NA x1 | |
| PEM-RLI ND x1 | |
| PEM-RLI NQD 1x | |
| PEM-RLI x2 | dimeric RLI2 constructs; |
| PEM-RLI KAQD x2 | with Fc of PEM |
| PEM-RLI DA x2 | IL-15 with indicated mutations (bold) |
| PEM-RLI NA x2 | |
| PEM-RLI ND x2 | |
| PEM-RLI NQD x2 | |
| PEM L-RLI x1 | monomeric RLI2 constructs; |
| PEM Y-RLI x1 | IL-15 wt |
| PEM LY-RLI x1 | PEM Fc: KIH + underlined mutation(s) |
| PEM L-RLI N65A x1 | monomeric RLI2 constructs; |
| PEM Y-RLI N65A x1 | IL-15 mutant N65A |
| PEM LY-RLI N65A x1 | PEM Fc with KIH + underlined mutation(s) |
| PEM L-RLI x2 | dimeric RLI2 constructs; |
| PEM Y-RLI x2 | IL-15 wt |
| PEM LY-RLI x2 | PEM Fc with under lined mutation(s) |
| PEM L-RLI N65A x2 | dimeric RLI2 constructs; |
| PEM Y-RLI N65A x2 | IL-15 mutant N65A |
| PEM LY-RLI N65A x2 | PEM Fc with under lined mutation(s) |

Immunocytokines and controls of Table 9 were tested for their predicted stability by measuring their melting temperatures (Tm) using differential scanning fluorimetry (DSF), which uses a real-time PCR instrument to monitor thermally induced protein denaturation by measuring changes in fluorescence of a dye that binds preferentially to unfolded protein (such as Sypro Orange, which binds to hydrophobic regions of proteins exposed by unfolding and water strongly quenches its fluorescence). This experiment is also known as a Protein Thermal Shift Assay, because shifts in the apparent melting temperature can be measured upon the addition of stabilizing or destabilizing binding partners or buffer components. Briefly, SYPRO 50X, prediluted in ultra-pure water (UPW), the protein sample and water are mixed to obtain a 25 μL-reaction sample at 5 to 10 μM of final protein concentration in SYPRO 5×. A negative control with SYPRO diluted to 5× final concentration with only UPW, and same mix with lysozyme 10 μM final concentration for positive control are done. Each mix of 25 μL is made in triplicate in a PCR plate and a specific program of thermocycling is running. This program has been created to get the best resolution as possible with our thermocycler. Melt Curves are drawn from 20.0° C. to 95.0° C., with an increment of 0.2° C. each 20 seconds. No fluorescence signal must be measured in negative control and only one peak at 70° C.±1° C. must be detected in positive control. To determinate buffer compatibility same controls are done with buffer instead of UPW and same results are expected. The derivative of the fluorescence versus temperature curve is used to determine the Tm of the protein, defined as the temperature at which 50% of a protein sample is in the folded state and 50% is in the unfolded state.

$$\frac{- d ( R F U )}{d T} = f ( T )$$

RFU: Relative Fluorescence Unit
T: Temperature
Tm corresponds to the negative peak of the drawn curve. The presence of several negative peaks is a sign that the protein has several levels of instability.

A decrease in melting temperature of 1.5° C. was observed when the KIH mutation is present (60.1° C. vs 61.6° C. for PEM WT). The KIH mutation on the Fc domain of pembrolizumab, without RLI coupling, induced a decrease in the stability of the antibody. There was a second melting temperature observed which is between 69° C. and 71° C. for all constructs. As this Tm is present in non-RLI coupled constructs, it corresponds to the denaturation of a very stable domain of the PEM antibody.

As expected, IL-15 mutants had no impact on the melting temperature of the tested immunocytokines.

A significant decrease in Tm was observed as a function of the mutations present on the Fc of PEM. The L (LE) mutation induced a 0.6° C. to 1.8° C. decrease in Tm compared to the non-mutated construction, whereas the Y (YTE) mutation induced a decrease of 5° C. to 6.5° C. The double mutation LY seems to combine the effect of the 2 mutations since the decrease could reach up to 7° C. to 9° C. compared to the non-mutated construction. The Tm dropped from 60° C. for PEM-RLI N65A x1 to 52° C. for PEM LY-RLI N65A x1 and from 61° C. for the non-muted PEM construct to 53° C. for PEM LY-RLI N65A x2.

Immunocytokines based on Rituximab were made comparing the RLI2_{AQ} fused to both heavy chains with (SEQ ID NO: 32) or without (SEQ ID NO: 33) the L40 linker (SEQ ID NO: 31) and identical light chains (SEQ ID NO: 34) showing no significant biological differences (data not shown).

### 8. PEM L-RLI NAx1 Molecule Enhances IFN-γ Production in Mixed Lymphocytes Reaction Over Pembrolizumab

PEM L-RLI N65A x1 was evaluated for its potential to enhance T-cell activation and IFNγ production using a mixed lymphocyte reaction (MLR). MLR is an in vitro assay in which leukocytes, from two genetically distinct individuals of the same species, are cocultured resulting in cell blast transformation, DNA synthesis and proliferation. Generation of the MLR occurs as a consequence of the incompatibility of the allogeneic determinants, which are expressed on the surface of cell populations, and which are encoded by the major histocompatibility complex (MHC). For the reaction, buffy coats from healthy donors were obtained. PBMC were isolated by Ficoll Paque gradient, washed three times. Pairs of hPBMCs donors were cultivated with equimolar concentration of pembrolizumab and PEM L-RLI-NA x1 at 1 nM for six days. IFNγ production in cell supernatants was determined by using human IFN-γ DuoSet ELISA (R&D systems, No. DY258B). Data are expressed as relative response of IFNγ production [%] and represent mean±SEM from—12 pairs of hPBMC healthy donors.

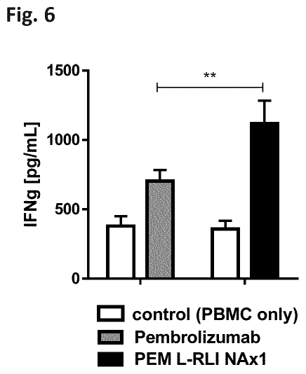

IFNγ production increased when mismatched human PBMC donor pairs were incubated with PEM L-RLI NA x1 (1 nM) (IL-15 with N65A and AQ mutation) in comparison to an equimolar amount of pembrolizumab (see FIG. 6). The data represent mean±SE of 12 donor pairs for pembrolizumab and PEM L-RLI NAx1. These data suggest a superior mechanistical action of PEM L-RLI NAx1 over pembrolizumab in IFN-γ stimulation from T cells.

### 9. PEM-RLI NAx1 Molecule Display Anti-Tumor Efficacy in Mouse Tumor Model

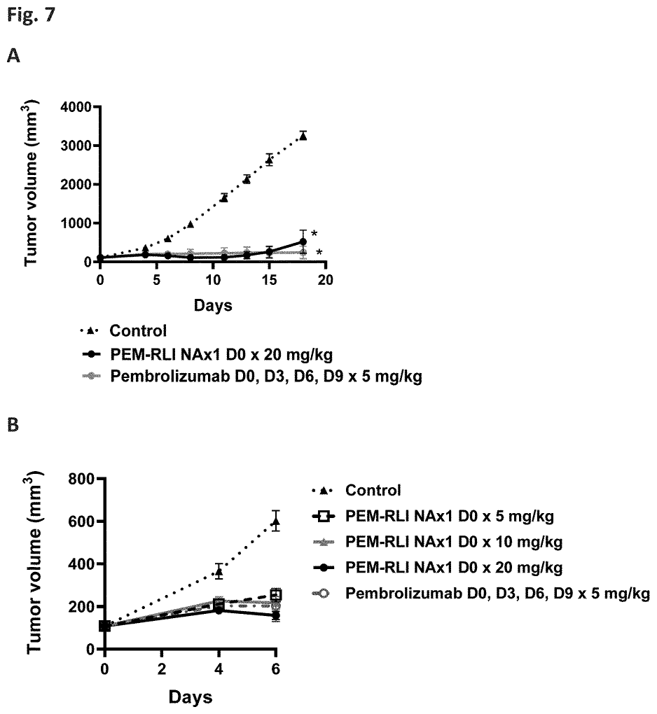

The objective of this study is to evaluate preclinically the in vivo therapeutic efficacy of test the construct PEM-RLI NAx1 and pembrolizumab as a monotherapy in the treatment of HuCell MC38-hPD-L1 cell line implanted in female hPD1 single KI HuGEMM mice (n=8 mice per group). The treatment started when the mean tumor size reached 108 mm³ at randomization day 0. PEM-RLI NA x1 (IL-15 with N65A and AQ mutation) was administered IV at 20 mg/kg at day 0 and pembrolizumab was administered IP at 5 mg/kg at days 0, 3, 6 and 9. Tumor observation was followed for 18 days (FIG. 7A). Concomitantly to this, PEM-RLI NA x1 (IL-15 with N65A and AQ mutation) was administered IV at 5, 10 at day 0. Tumor observation was followed for 6 days (FIG. 7B).

A single dose of PEM-RLI NA x1 strongly decreased tumor volume in this model in comparison to the control untreated group (p-value was <0.05) and similarly to multiple doses of pembrolizumab (see FIG. 7A). The tumor decrease with PEM-RLI NA x1 is observed also for the lower dose 5 mg/kg after a single administration in comparison to multiple doses of pembrolizumab (see FIG. 7B)

### 10. IL-15 Muteins for Reduced In Vitro Potency

Mutations were introduced within the IL-15 part of the RLI2 conjugate in order to reduce the binding and thereby the in vitro potency of the RLI conjugate to the IL-2Rβ and/or γ receptor, and to reduce heterogenicity of the RLI2-containing products. Indicated amino acid substitutions were made in the mature human IL-15 sequence (see Table 10).

TABLE 10
Amino acid substitutions in IL-15 and respective position in RLI2

| mature human IL-15 position | RLI2 position |
|---|---|
| D8A | D105A |
| K10A | K107A |
| D30N | D127N |
| D61A | D158A |
| E64Q | E161Q |
| N65D | N162D |
| N65A | N162A |
| G78A | G175A |
| N79Q | N176Q |
| Q101D | Q198D |
| Q108A | Q205A |

TABLE 11
Potency of RLI2 IL-2/IL-15Rβγ muteins
having IL-15 variants on kit225 cells

EC50 kit225

| Variant/Substitution(s) | pM | lower potency |
|---|---|---|
| RLI2_{AQ} | 35 | 1x |
| K10A | 35 | 1x |
| Q101A | 32 | 0.9x |
| Q101D | 40 | 1.1x |
| D61A | 272 | 7.7x |
| N65D | 691 | 19.7x |
| N65A | 1,692 | 48.3x |
| NQD | 23,505 | 671.5x |
| D8A | 0 | 0 |

Tested IL-15 substitutions affecting the binding to the IL-2/IL-15Rβ and/or γ markedly reduced the potency of the RLI molecule on kit225 cells. The single mutant N65A lead to the most significant decrease, but lower than triple mutant NQD. (see Table 11). Other substitutions only had a minor influence on the potency.

TABLE 12
Potency of RLI-15 muteins (as such, without
being fused to an antibody) on kit225 cells

| | EC50 kit225 | |
|---|---|---|
| Sample | cells [pM] | |
| | | Relative potency to |
| | | SOT101 |
| SOT101 | 43.58 | — |
| RLI KAQD (AD) | 84.09 | 51.83% |
| RLI DA | 451.2 | 9.66% |
| RLI ND | 473.4 | 9.21% |
| RLI NA | 3847 | 1.13% |
| | | Relative potency to |
| | | EM-RLI NQD x1 |
| PEM-RLI NQD x1 | 21617 | — |
| RLI DANA | 38619 | 55.98% |
| RLI DANAQD | 105930 | 20.41% |

Also for RLI-15 muteins tested without being bound to an antibody, the NA mutation lead to an about 2 log reduction of activity, here measured as EC50 on kit 225 cells.

### 11. IL-15 N65A Mutation in a PD-1-Targeted Immunocytokine Shows Diminished Potency on Kit225 Cells

Immunocytokines based on the anti-PD-1 antibody pembrolizumab were generated in various formats comprising an RLI molecule. Pembrolizumab is a humanized IgG4-K antibody having the stabilizing S228P mutation in the Fc part of the antibody. Variations of pembrolizumab (“PEM”) were tested in order to improve the construct for the use in an immunocytokine. Although the IgG4 antibody class is known to have relatively low ADCC activity, the L235E mutation (Alegre et al. 1992) (“LE” or short “L”) was introduced in order to further reduce ADCC (SEQ ID NO: 28). More complex ADCC inactivating mutations were avoided in order to limit the potential of immunogenicity/anti-drug antibodies. Either one or two RLI2 molecules were genetically fused to the C-terminus of the PEM antibody. In case of homodimeric PEM variants (“x2”) one RLI2 molecule was fused to each heavy chain, whereas heterodimeric PEM variants (“x1”) were made using the knob-in-hole (KiH) technology (Elliott et al. 2014), whereas one RLI2 molecule was fused to the knob heavy chain having the T336W substitution (SEQ ID NO: 26) having additionally the L235E mutation for reducing ADCC activity, whereas the hole heavy chain (with no RLI2 fusion) comprised the T366S/L368A/Y407V substitutions (SEQ ID NO: 27), also having the additional L235E mutation. When RLI2 was fused to a heavy chain, the terminal lysine (K) was deleted (“dK”) in order to reduce heterogeneity of the product. Further, different RLI2 muteins were used to fuse to the heavy chain of the antibody. All RLI2 molecules had the AQ (G78A/N79Q) substitution for reducing the heterogeneity of the product, and the following substitutions reducing the binding of RLI2 to the IL-2/IL-15Rβγ were tested in the PEM-RLI immunocytokines: KAQD, DA, NA, ND, and NQD. Made PEM-RLI immunocytokines are listed in Table 13, left column. An exemplary PEM-RLI heterodimeric immunocytokine SOT201 was made using the sequences of SEQ ID NO: 22 (HC knob: IgG4 S228P.L235E.T366W.dK-RLI2.N162A.G175A.N176Q-RLI2_{AQ} N162A), SEQ ID NO: 23 (HC hole: S228P.L235E.T366S.L368A.Y407V), and SEQ ID NO: 24 (LC).

The potency of several homodimeric or heterodimeric PEM-RLI2_{AQ} immunocytokines with the provided IL-15 substitutions was compared by measuring the in vitro EC50 on kit225 cells (Table 13) with RLI2 being used as a standard and set to 100% for relative potency. EC50 was calculated using GraphPad Prism 8.4.3. The aim was to identify the least potent mutein of RLI-15 on kit225 cells. Shown results are mean of 2-5 experiments.

TABLE 13
Potency of PEM-RLI2 mutants on kit225

| | No. of RLI | EC50 kit225 | Relative potency/ |
|---|---|---|---|
| Sample | molecules | cells pM | Kit225 cells |
| RLI | 1x | 51 | 100% |
| PEM-RLI x1 | 1x | 335 | 15% |
| PEM-RLI x2 | 2x | 109 | 47% |
| PEM-RLI-KAQD x1 | 1x | 603 | 8.5% |
| PEM-RLI-KAQD x2 | 2x | 122 | 42% |
| PEM-RLI-DA x1 | 1x | 4484 | 1.1% |
| PEM-RLI-DA x2 | 2x | 1620 | 3.1% |
| PEM-RLI-NA x1 | 1x | 13983 | 0.36% |
| PEM-RLI-NA x2 | 2x | 10663 | 0.47% |
| PEM-RLI-ND x1 | 1x | 9238 | 0.55% |
| PEM-RLI-ND x2 | 2x | 5270 | 0.97% |
| PEM-RLI-NQD x1 | 1x | ND | — |
| PEM-RLI-NQD x2 | 2x | 116988 | 0.044% |

RLI: RLI2_{AQ};
ND . . . not detected (limited sensitivity of the assay)

The RLI2_{AQ}NA within PEM-RLI-NA x1 was identified as the least potent RLI mutein with a single mutation lowering the IL-2/IL-15Rβγ, which still is about 10fold more active than the NQD mutation, which has three amino acid substitutions, thereby having a relatively higher risk of immunogenicity.

### 12. Evaluation of Low Potency PEM-RLI Mutants Attached to HC or LC on Kit225 Cells In Vitro

Several low potency IL-5 muteins in the PEM-RLI immunocytokines with or without mutated Fc antibody part (LE-YTE, or short “LY”: LE for the Fc mutation L235E according to EU numbering of an IgG4 antibody to reduce ADCC activity of the Fc domain; YTE for the Fc mutation M252Y/S254T/T256E according to EU numbering reported to enhance FcRn binding to enhance the in vivo half-life) were compared with respect to their potency in comparison to PEM LE/YTE-RLI NA x1 as a reference. In “LC” immunocytokines, the RLI conjugate was fused to the C-terminus of the light chains of the antibody without a linker (see SEQ ID NO: 30 plus indicated IL-15 substitutions DA, NA and DANA), whereas all other constructs have the RLI conjugate fused to the C-terminus of one of both heavy chains. The in vitro potency testing was accomplished using kit225 cell line with an altered protocol (prolonged cell incubation). The potency of molecules was assessed as EC50 and also calculates as a relative potency related to the PEM LE/YTE-RLI NA x1 molecule. The data represent mean of 2-4 experiments.

TABLE 14
Potency of PEM-RLI2AQ molecules on kit225; Lc for light chain fusions

| | No. of | EC50 | Relative |
|---|---|---|---|
| | RLI2 | (kit225 cells) | potency/ |
| | molecules | pM | kit225 cells |
| PEM LE/YTE -RLI2 NA x1 | 1x | 4 756 | 100% |
| PEM -RLI2 NQD x1 | 1x | 64 758 | 7.3% |
| PEM LE/YTE -RLI2 QDQA x1 | 1x | 9 449 | 50.3% |
| PEM LE/YTE -RLI2 DANA x1 | 1x | 480 157 | 1.0% |
| PEM LE/YTE -RLI2 DANAQD x1 | 1x | NA | NA |
| PEM LE/YTE-Lc-RLI2 DA x2 | 2x | 657 | 724% |
| PEM LE/YTE-Lc-RLI2 NA x2 | 2x | 3 493 | 136% |
| PEM LE/YTE-Lc-RLI2 DANA x2 | 2x | 272 966 | 1.7% |

The combinations of substitutions QDQA (Q101D/Q108A), NQD (D30N/E64Q/N65D), DANA (D61A/N65A) and DANAQD (D61A/N65A/Q101D) further reduced potency of the PEM-RLI immunocytokine constructs until not measurable for the DANAQD construct. Immunocytokines with the RLI conjugate fused to the light chains of the antibody showed similar potency compared to the constructs with only one RLI conjugate having the same IL-15 mutations on one heavy chain of the antibody.

### 13. Analysis of Anti-Drug Antibodies of PEM-RLI Immunocytokines in Cynomolgus Monkeys

Cynomolgus monkeys were administered with 0.3 mg/kg of the indicated PEM-RLI immunocytokine according to the scheme as depicted in Table 15.

TABLE 15
dosing scheme of cynomolgus monkey PK/PD study

| | | | Day 1 | Day 15 | Day 22 | Day 29 |
|---|---|---|---|---|---|---|
| | Group | admin | mg/kg | mg/kg | mg/kg | mg/kg |
| PEM LY-RLI2 NA x1 | A | IV | 0.6 | 0.3 | — | 0.3 |
| PEM LY-RLI2 NA x1 | B | SC | 0.6 | 0.3 | — | 0.3 |
| PEM L-RLI2 NA x1 or | C | IV | 0.6 | 0.3 | 0.3 | 0.3 |
| PEM LY-RLI2 NA x1 | | | | | | |
| PEM RLI2-NQD x1 | D | IV | 0.6 | 0.3 | — | 0.3 |

For group C: admin. At day 1 with PEM L-RLI2 NA x1, at days 15, 22 and 29 with PEM LY-RLI2 NA x1.

ADA titers were measured from serum taken at day 15 and determined by ELISA. Neutralizing antibodies were determined by FACS analysis of STAT5 phosphorylation by the serum samples from tested PEM-RLI immunocytokines in kit225 cells.

TABLE 16
Anti-drug antibodies (ADA) and neutralizing antibodies (NAb) after
IV or SC administration of indicated PEM-RLI immunocytokines
after IV or SC administration in cynomolgus monkeys at day 50

| | | | ADA | | | NAb |
|---|---|---|---|---|---|---|
| | admin. | monkey | αPEM-RLI | αPEM | αRLI2 | αRLI2 |
| PEM LY-RLI2 NA x1 | IV | A1 | + | + | − | − |
| | | A2 | + | + | − | − |
| | | A3 | + | + | + | + |
| PEM LY-RLI2 NA x1 | SC | B4 | + | + | + | +++ |
| | | B5 | + | + | + | + |
| | | B6 | + | + | + | ++ |
| PEM L-RLI2 NA x1 or | IV | C7 | + | + | − | − |
| PEM LY-RLI2 NA x1 | | C8 | + | + | + | − |
| | | C9 | + | + | − | − |
| PEM RLI2-NQD x1 | IV | D10 | + | + | + | + |
| | | D11 | + | + | + | − |
| | | D12 | + | + | + | + |

As pembrolizumab is known to induced ADAs in cynomolgus monkeys, it was not surprising that all monkeys generated ADAs against the tested immunocytokines, and all monkeys developed ADAs that could be shown to be reactive against the antibody part of the immunocytokine (αPEM column). Both groups with the RLI2_{AQ}NA single substitution (IV administration) had only one monkey developing ADAs against the RLI part, whereas all monkeys of the RLI2_{AQ}NQD triple substitution generated ADAs against RLI. Subcutaneous administration of the PEM LY-RLI NA x1 immunocytokine also generated ADAs in all monkeys.

It was further demonstrated that generated ADAs partially were neutralizing and therefore potentially limiting the therapeutic efficacy of the immunocytokine, especially for multiple administrations. Again, the NQD triple substitution was inferior to the NA single substitution (IV), whereas SC administration generated the highest amount of neutralizing antibodies.

ADA and nAB development against the RLI-part of the immunocytokine correlated with the pharmacodynamic response in the monkeys, as monkeys dosed with the PEM-RLI2 NQD (and SC administered monkeys) showed a marked reduction of restimulation of lymphocytes after day 29 compared to stimulation after day 1 and day 15, whereas IV administered immunocytokines with the RLI2_{AQ} NA mutein still showed strong lymphocyte proliferation (data not shown). The loss of the ability to be restimulated may be due to the neutralizing antibodies.

### 14. PEM-RLI NA x1 Immunocytokine Displays Anti-Tumor Efficacy in Mouse Tumor Model

The in vivo therapeutic efficacy of the PEM-RLI NA x1 immunocytokine was compared to Pembrolizumab as monotherapy in the treatment of HuCell MC38-hPD-L1 tumor cell line implanted in in female hPD-1 single KI HuGEMM mice (n=8 mice per group). The treatment started when the mean tumor size reached 108 mm³ at randomization day 0. PEM-RLI NA x1 was administered IV at 20 mg/kg at day 0 and pembrolizumab was administered IP at 5 mg/kg at days 0, 3, 6 and 9. Tumor observation was followed for 18 days. Concomitantly to this, PEM-RLI NA x1 (IL-15 with N65A and AQ mutation) was administered IV at 5, 10 at day 0. Tumor observation was followed for 6 days.

PEM-RLI NA x1 strongly decreased tumor volume in this model in comparison to the control untreated group (p-value was <0.05) and similarly to the pembrolizumab treatment group (see FIG. 7). While no marked difference to pembrolizumab was seen for the immunocytokine, it should be noted that a single injection of the immunocytokine achieved a similar result as four administrations of pembrolizumab. Moreover, lower dose of 5 mg/kg was similarly efficient. Further, as mouse is known to be about 10fold less sensitive to RLI, the full functionality of the PEM-RLI NA x1 cannot be tested in this mouse model and accordingly treatment effect in humans is expected to be better.

15. ADCC Activity of Immunocytokines Based on Anti-Claudin18.2 hCl1a Antibody with Modified Effector Functions.

### Cell Lines:

Human cell lines PA-TU-8988S (Creative Bioarray, catalog number CSC-C0326) and A549 (ATCC CCL-185) overexpressing Claudin 18.2 (A549-Cldn18.2) were grown in DMEM medium (Gibco) supplemented with 10% fetal bovine serum, 2 mM glutamine (GlutaMAX, Gibco), 100 U/ml penicillin, 0.1 mg/ml streptomycin (Invitrogen) and 2 μg/ml puromycin (Gibco).

A549 cells were co-transfected by electroporation with a transposase expression construct (pcDNA3.1-hy-mPB), a construct bearing transposable full-length huCLDN18.2 (pPB-Puro-huCLDN18.2) along with a puromycin resistance cassette and a construct carrying EGFP as transfection control (pEGFP-N3) (Waldmeier et al. 2016). Upon electroporation, cells were allowed to recover for two days in growth media at 37° C. in a humidified incubator in 5% CO2 atmosphere. Transfection was verified by FC analysis of the EGFP expression. Cells expressing CLDN18.2 were then selected by the addition of puromycin into culture at 1 μg/ml, and further expanded to allow the generation of frozen stocks in FCS with 10% DMSO. The expression of CLDN18.2 in the transfected cells was analyzed by FC.

In order to have a more homogenous PA-TU-8988S cell population, the cells were sorted by FACS to select only cells with a the higher CLDN18.2 expression. In brief, PA-TU-8988S cells suspended in FACS buffer (PBS, 2% FCS) were incubated on ice for 30 min with Zolbetuximab at 2 μg/ml. After wash in FACS buffer, the cells were incubated with the PE-labeled Fcγ specific IgG goat anti-human secondary antibody (eBioscience) on ice for 30 min. After wash, the stained cells were resuspended in FACS buffer, analyzed and sorted by a FACSAria™ instrument, separating medium expressing cells from high expressing cells. After sorting the collected PA-TU-8988S-High cells (PaTu) were resuspended in growth media, expanded and frozen aliquots were preserved in liquid N2.

The human NK cell line NK92 (ATCC CRL-2407) exogenously expressing human CD16 (NK92-hCD16, here referred to as NK92) was generated as described in Clemenceau et al. (2013). The cells were grown in RPMI 1640 medium (Gibco) supplemented with 10% AB human serum (One Lambda), 2 mM glutamine (GlutaMAX, Gibco) and 5 ng/ml IL-2 (Peprotech). All cells were maintained at 37° C. in a humidified atmosphere containing 5% CO₂.

Human NK cells were isolated from fresh blood from healthy donors and diluted in a 1:1 ration with cold PBS-EDTA, ph7.4 and PBMC were isolated by Ficoll-Paque gradient isolation. Isolated PBMCs were resuspended in complete culture medium. hNK cells were isolated from a the PBMC using the EasySep Human NK Cell Isolation kit (Stem Cell Technologies, USA) according to the manufacturer instructions. Isolate hNK cells of each donor were resuspended in NK medium with 10% serum at a concentration of 3×10⁶ cells/ml.

### Cell Based ADCC Assay:

A549-Cldn18.2 or PaTu cells were seeded into 96-well plates at an appropriate concentration (A549-Cldn18.2—20.000 cells, PaTu—30.000 cells) and incubated for 24 h. NK92 cells or isolated human NK cells were collected by centrifugation, washed and resuspended in ADCC assay medium (RPMI 1640 (no phenol red) supplemented with 2 mM glutamine and 10% heat-inactivated (56° C. for 20 min) pooled complement human serum (Innovative Research)). The medium from 96-well plates containing adhered cells (target cells T) was removed and NK92 cells in suspension in the ADCC assay medium (effector cells E) were added to the adherent target cells at an E:T ratio of 10 for A549-Cldn18.2 and of 5 for PA-TU-8988S cells. Antibodies or immunocytokines (ICK) to be tested were added in a concentration range of 0.001-100 nM or 0.0001-10 μg/ml. A human IgG1 isotype antibody (Ultra-LEAF™ Purified Human IgG1 Isotype Control Recombinant Antibody, Biolegend, cat. no. 403502) was included as an unspecific control. The mixture was incubated over-night at 37° C. After 24 h, cytotoxicity was measured, expressed as the activity of lactate dehydrogenase enzyme released from dead cells, using the LDH Cytotoxicity Assay (Abcam, ab65393) according to manufacturer's instructions: 10 l of supernatant was transferred into a new 96-well plate, mixed with the LDH substrate and the developed colour change was measured using spectrophotometer at an OD of 450 nm. Cytotoxicity was calculated according to this formula: Cytotoxicity (%)=((Test Sample−effector cell control−low control)/(High Control−low control))×100; “test sample”: effector/target mix; “effector cell control”: one well with NK92 cells only (determines LDH activity released from effector cells); “low control”: one well with target cells only (determines a spontaneous release of LDH activity form untreated target cells); “high control”: one well with target cells permeabilized with lysis buffer (determines the maximal releasable LDH activity).

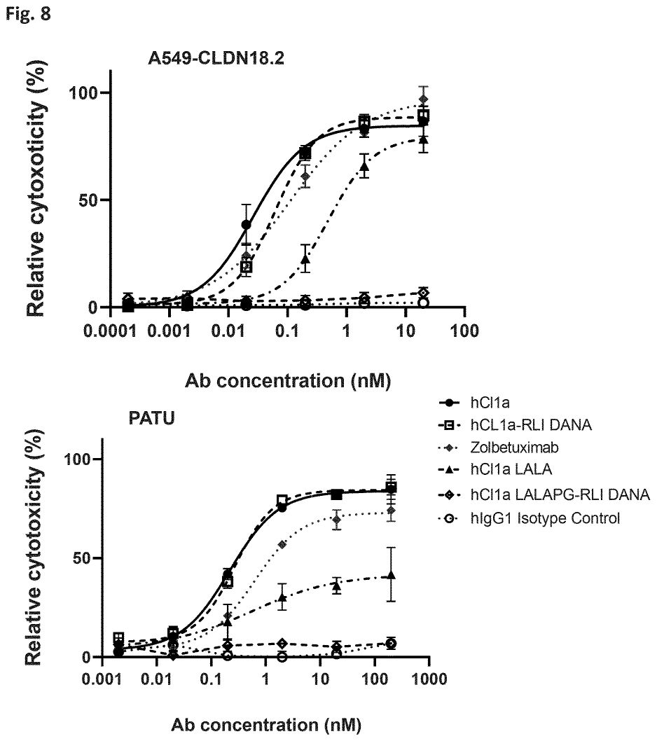

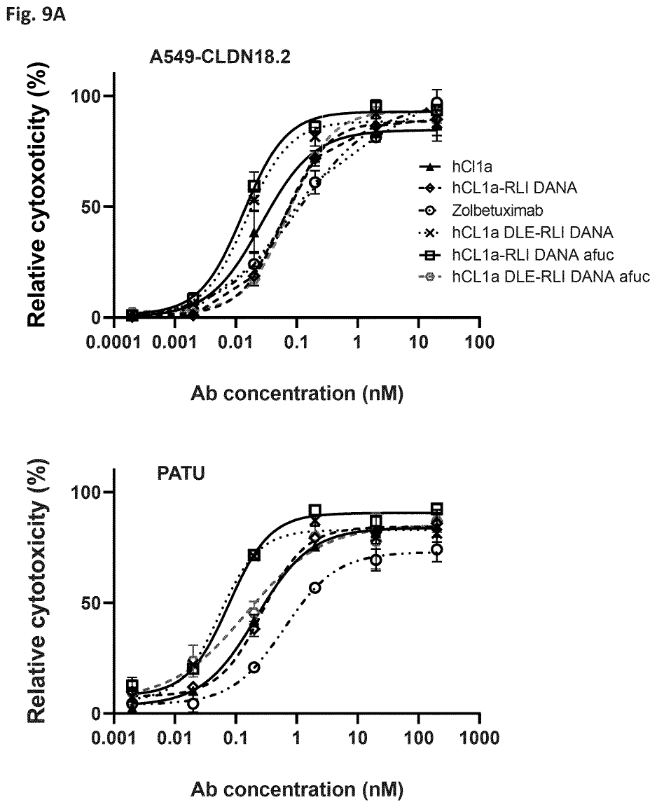

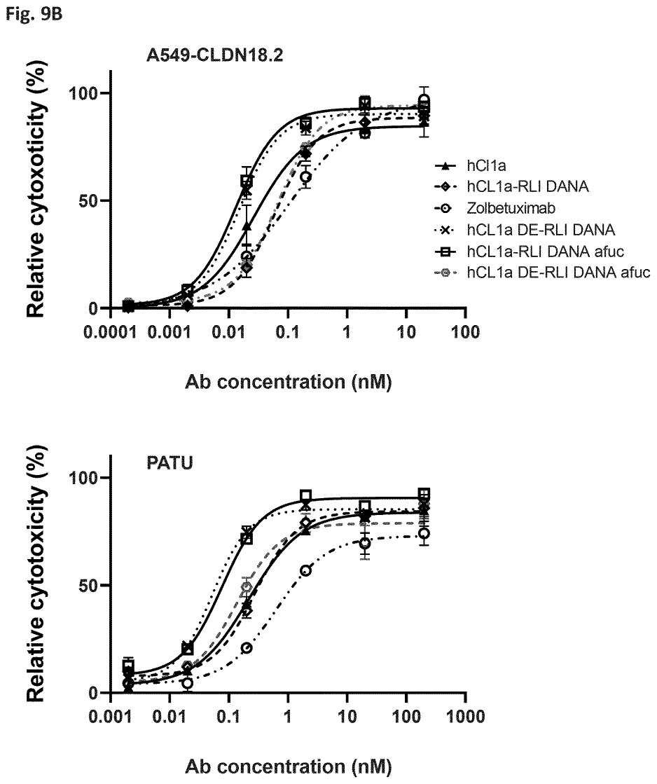

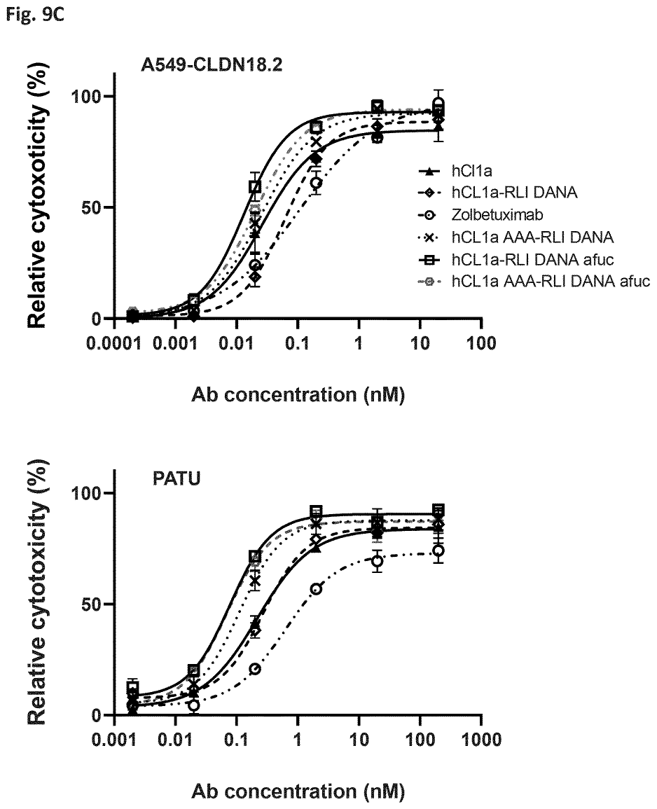

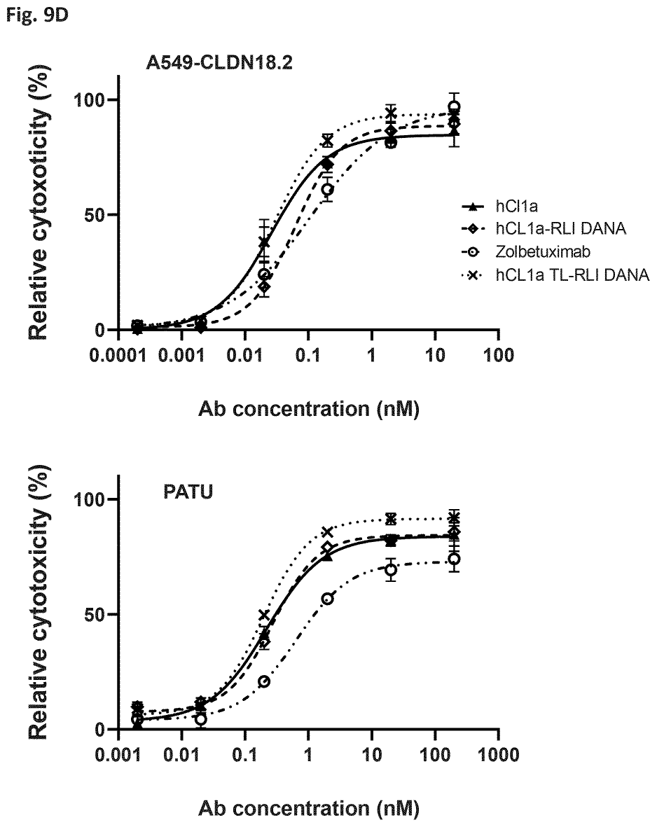

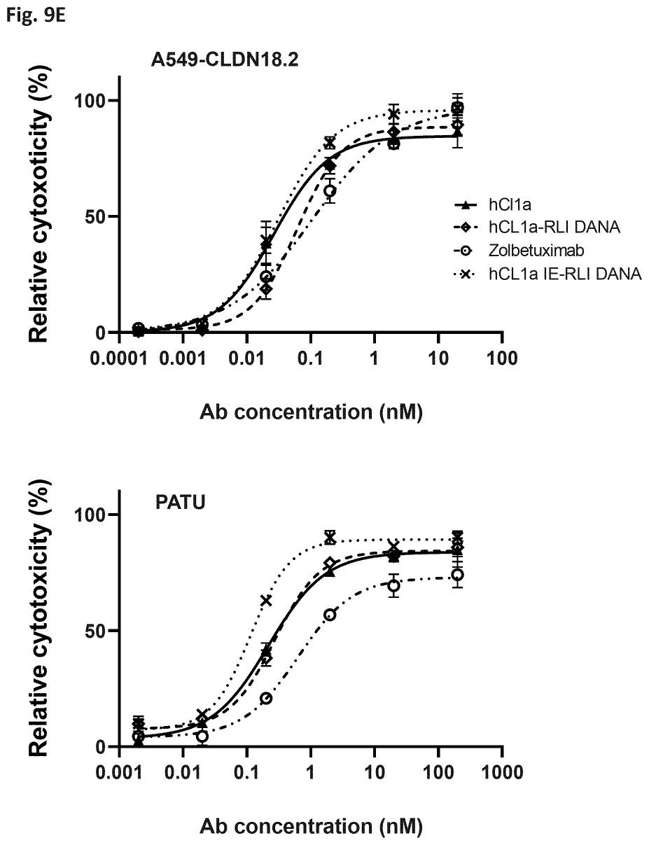

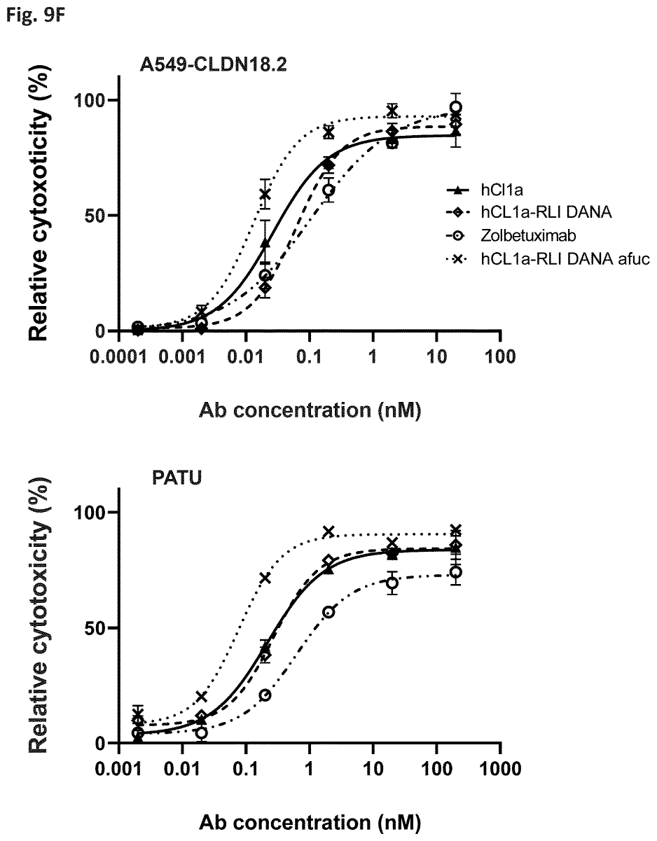

FIG. 8 show the ADCC activity of immunocytokines based on the hCl1a antibody with modified effector function. All the tested immunocytokines had heterodimeric Fc domains, with one RLI2_{AQ} conjugate fused to the C-terminus of one of the heavy chains. An exemplary immunocytokine directed against Claudin18.2 is built from SEQ ID NO: 35 (hCl1a heavy chain knob with AAA mutation fused to RLI2_{AQ} NA), SEQ ID NO: 36 (hCl1a heavy chain hole) and SEQ ID NO: 37 (hCl1a light chain). When immunocytokines with mutations of effector domain reducing ADCC were tested, the immunocytokine hCl1a LALAPG-RLI DANA showed nearly abolished ADCC activity when tested on A549-CLDN18.2 cells (upper panel) or PA-TU-8988S (lower panel) in the presence of NK92 cells, when compared to the hCl1a-DANA immunocytokine of hCl1a antibody alone. The hCl1a-LALA antibody showed also reduced ADCC activity when compared to the hCl1a antibody, however the ADCC activity was not fully abolished. The addition of the conjugate did not affect the ADCC activity of the immunocytokines when ADCC activity was reduced, when compared to the ADCC activity of the antibody alone. Table 17 recapitulates the ADCC EC50 values measured for each tested immunocytokine or antibody. The EC₅₀ values were determined using the Graphpad Prism Software with the built-in “log(AGONIST) vs. response−variable slope (four parameters)” EC50 determination. When immunocytokines with mutations of effector domain enhancing ADCC were tested, all the tested immunocytokines based on the hCl1a antibody with DLE, DE, AAA, TE or IE mutations in the Fc domain showed enhanced ADCC activity, when compared to the same immunocytokine without those mutations or the antibody alone (FIG. 9).

Afucosylation was also tested to enhance ADCC activity. FIG. 9F shows that, in A549-Cldn18.2 and PA-TU-8988S cells, the afucosylated immunocytokine hCl1a-DANA afuc has enhanced ADCC activity when compared to hCl1a-DANA, and comparable ADCC activity to the immunocytokines with the DE and DLE mutations described above. However, when afucosylation was combined with mutations of effector domain enhancing, afucosylation surprisingly negatively affected the ADCC enhancement induced by the DE or DLE mutations (see FIGS. 9B and A). Nevertheless, enhanced ADCC activity was maintained when afucosylation was combined with the AAA mutations (FIG. 9C)

TABLE 17
ADCC EC50 values for tested immunocytokines
and antibodies (RLI = RLI2_{AQ})

| | EC50 | |
|---|---|---|
| Antibody or ICK | A549-CLDN18.2 | PA-TU-8988S |
| Zolbetuximab | 0.106 | 0.628 |
| hCl1a | 0.02653 | 0.2196 |
| hCl1a LALA | 0.4899 | Not measurable |
| hCl1a LALAPG | Not measurable | 0.5635 |
| hCl1a-RLI DANA | 0.062 | 0.274 |
| hCl1a DLE-RLI DANA | 0.01514 | 0.05339 |
| hCl1a DE-RLI DANA | 0.0149 | 0.05198 |
| hCl1a AAA-RLI DANA | 0.02535 | 0.1116 |
| hCl1a TL-RLI DANA | 0.03036 | 0.1961 |
| hCl1a IE-RLI DANA | 0.03055 | 0.1157 |
| hCl1a-RLI DANA afuc | 0.01332 | 0.07711 |
| hCl1a DLE-RLI DANA afuc | 0.07504 | 0.1519 |
| hCl1a DE-RLI DANA afuc | 0.06663 | 0.1378 |
| hCl1a AAA-RLI DANA afuc | 0.02034 | 0.06905 |

### 16. Evaluation of Antibody Fc Binding to ADCC-Activating Receptors FcγIIIa V158, and FcγRIIIa F158 and ADCC-Inhibitory Receptor FcγRIIb by Surface Plasmon Resonance (SPR)

The human FcγRIIIa receptor (hFcγRIIIa; CD16a) exists as two polymorphic variants at position 158, hFcγRIIIaV 158 and hFcγRIIIaF158. FcγRIIIa activates ADCC activities, while FcγRIIb inhibits ADCC. The ADCC activity of the immunocytokines, when their affinity to the receptor is measured by SPR, can be expressed as the ratio of the EC50 binding affinity to FcγRIIIa to the EC50 binding affinity to FcγRIIb.

SPR experiments were performed on a Biacore 8K (Cytiva, Chicago, IL, USA), using CM5 sensor chips (Cytiva) with an immobilization using THE His tag antibodies (Genscript). FcγRIIIa V158, FcγRIIIa F158 or FcγRIIb protein were used for capture with a contact time of 30 sec at a flow rate of 10 μl/mm in a 1×HBS-EP+ running buffer. Association/dissociation rates were measured for each tested immunocytokine at a flow rate of 30 μl/min with concentration serial dilution in a suitable range with an association time/dissociation time of 300 s/300 s except for constructs with DLE and DE with and without afucosylation, where association/dissociation time of 120 s/1200 s was applied. Table 18 below summarizes the results of the SPR measurements.

TABLE 18
SPR data (RLI = RLI2_{AQ})

| | Kd FcγRIIIa | Kd FcγRIIIa | | | |
|---|---|---|---|---|---|
| Antibody or | V158 - | F158 - | Kd | A/I | A/I |
| immunocytokine | high affinity | low affinity | FcγRIIb | (V158) | (F158) |
| Zolbetuximab | 8.64 × 10⁻⁸ | 4.23 × 10⁻⁷ | 1.39 × 10⁻⁵ | 160.9 | 32.9 |
| hCl1a | 1.1 × 10⁻⁷ | 4.48 × 10⁻⁷ | 9.22 × 10⁻⁶ | 83.8 | 20.6 |
| hCl1a-RLI DANA | 9.91 × 10⁻⁸ | 3.81 × 10⁻⁷ | 6.58 × 10⁻⁶ | 66.4 | 17.3 |
| hCl1a DLE-RLI DANA | 5.71 × 10⁻⁹ | 1.16 × 10⁻⁸ | 2.65 × 10⁻⁶ | 464.1 | 228.4 |
| hCl1a DE-RLI DANA | 7.06 × 10⁻⁹ | 1.87 × 10⁻⁷ | 1.78 × 10⁻⁶ | 525.1 | 95.2 |
| hCl1a AAA-RLI DANA | 3.56 × 10⁻⁸ | 1.01 × 10⁻⁷ | 2.46 × 10⁻⁵ | 691.0 | 243.6 |
| hCl1a TL-RLI DANA | 4.76 × 10⁻⁸ | 1.39 × 10⁻⁷ | 2.9 × 10⁻⁶ | 60.9 | 20.9 |
| hCl1a IE-RLI DANA | 2.44 × 10⁻⁸ | 6.73 × 10⁻⁸ | 3.68 × 10⁻⁶ | 150.8 | 54.7 |
| hCl1a -RLI DANA afuc | 2.74 × 10⁻⁸ | 8.81 × 10⁻⁸ | 4.29 × 10⁻⁶ | 156.6 | 48.7 |
| hCl1a DLE-RLI DANA | 1.62 × 10⁻¹⁰ | 2.37 × 10⁻¹⁰ | 2.22 × 10⁻⁶ | 13703.7 | 9367.1 |
| hCl1a DE-RLI DANA | 1.85 × 10⁻¹⁰ | 3.74 × 10⁻¹⁰ | 1.7 × 10⁻⁶ | 9189.2 | 4545.5 |
| afuc | | | | | |
| hCl1a AAA-RLI | 1.73 × 10⁻¹⁰ | 4.61 × 10⁻⁸ | 1.75 × 10⁻⁵ | 1011.6 | 379.6 |
| DANA afuc | | | | | |

A/I ratio = (affinity towards FcgRIIIa)/(affinity towards FcgRIIb) were Affinity = 1/Kd.
“afuc” for afucosylated

The A/I ratio allows to evaluate the binding strength towards the ADCC-activating receptors (“A”; FcgRIII) compared to the binding strength towards the ADCC-inhibiting receptors (“B”; FcγRIIb). The higher the ratio, the stronger is the binding to the activating receptors of the antibody or immunocytokine.

The SPR data confirm that overall, all the immunocytokines with mutations enhancing ADCC show a higher A/I ration than the immunocytokine without mutations enhancing ADCC, a part of the TL mutations. The comparatively low A/I ratio for the TL mutations may be due to the increased glycosylation of such mutations (see example 17).

17. Stability/Developability of Immunocytokines Based on hCl1a with Enhanced ADCC Activity

Immunocytokines based on hCl1a having the DLE, DE, AAA, TL or IE mutation enhancing ADCC, or being afucosylated, where evaluated for their stability and developability, by evaluating the melting temperature of the C_{H}2 domain, sequence liabilities and glycosylation (N-Glycan) profiles.

Melting temperature of the C_{H}2 domain was measured by Differential scanning calorimetry (DSC) using a MicroCal PEAQ-DSC Automated system (Malvern Panalytical). In brief, the immunocytokine sample was diluted in its storage buffer to 1 mg/ml. The heating was performed from 20° C. to 100° C. at a rate of 1° C./min. Protein solution was then cooled in situ and an identical thermal scan was run to obtain the baseline for subtraction from the first scan.

For N-glycan analysis, the protein was firstly reduced with DTT, and then transfer to an HPLC column with glass-insert vial for injection. The protein was separated by reversed-phase chromatography and detected by Waters/XEVOG2XS-QTOF on-line LC-MS combined with UV detector. The molecular weight of detected glycan chains was matched with known N-glycan types, and the N-glycan relative abundance was calculated and represented by the intensity of the detected peaks.

Amino acid sequences of immunocytokine constructs bearing ADCC enhancement mutations were analysed for the presence of following additional sequence liabilities (not present in constructs without ADCC enhancement mutations) as described in Table 19.

TABLE 19
Known sequence liabilities.

| Sequence liabilities | Searched hotspots |
|---|---|
| N-glycosylation | NX[S/T] where X is any common |
| | amino acid except proline |
| Asparagine deamidation (Robinson | NG, NS, NN, NT, NH |
| and Robinson 2004, Lu et al. 2019) | |
| Aspartate isomerisation (Robinson | DG, DS, DD, DT, DH |
| and Robinson 2004, Lu et al. 2019) | |
| Unpaired cysteines | C |
| Methionine oxydation | M |

The TL mutation introduced a N-glycosylation sequence liability (mutation K392T in close proximity to N390 in the IgG1 sequence). No sequence liability was introduced by the other mutations (see Table 20).

TABLE 20
Stability and developability summary.

| | Sequence | | Melting |
|---|---|---|---|
| Modification | liabilities | N-glycans | temperature |
| WT | 4 | 4 | 4 |
| afucosylation | 4 | 4 | 4 |
| DLE | 4 | 4 | 2 |
| DE | 4 | 4 | 2 |
| AAA | 4 | 3 | 4 |
| TL | 1 | 1 | 4 |
| IE | 4 | 2 | 4 |

Score 4: Parameter is in the range expected for a mAb-based drug product;
Score 3: Careful monitoring/evaluation of quality attribute required during development;
Score 2: Considerable impact on timeline and/or cost is likely;
Score 1: High risk which cannot be controlled adequately.

Overall, afucosylation had no impact on stability and developability, and thus may be used to enhance ADCC activity of the immunocytokine. DLE and DE mutations caused a considerable decrease in Tm 1 (Melting temperature of the C_{H}2 domain) (see Table 21), potentially impacting the stability in solution of the immunocytokine. However, these mutations did not impact the glycosylation of the immunocytokines. The sequence liabilities introduced by the TL mutations resulted in the introduction of undesired sialylated and high mannose glycan species (see Table 22). These species may negatively impact the pK of the immunocytokines. Likewise, immunocytokines with the IE mutations had a high proportion of mannose species, potentially impacting their properties. Immunocytokines with the AAA mutations resulted in the increase of mannose species (see Table 22). However, production of afucosylated immunocytokine partially reverted the glycosylation to acceptable levels with regards to developability. Therefore, when enhancement of the immunocytokine based on hCl1a is desired, the AAA mutations, optionally combined with afucosylation, may be the recommended mutations affecting the least its stability and developability. Afucosylation had no impact on evaluated properties. DLE and DE mutations caused a considerable decrease in Tm, potentially destabilising the molecule. TL mutation introduced an additional glycosylation site into Fc. Construct with IE mutation had a high proportion of mannose species.

TABLE 21
Melting temperatures (“afuc” for afucosylated).

| Immunocytokine or antibody | Tm1 (° C.) |
|---|---|
| hCl1a | 69.5 |
| hCl1a-RLI DANA | 71.8 |
| hCl1a-RLI DANA afuc | 72.6 |
| hCl1a DLE-RLI DANA | 51.1 |
| hCl1a DLE-RLI DANA afuc | 51.3 |
| hCl1a DE-RLI DANA | 51.3 |
| hCl1a DE-RLI DANA afuc | 52.4 |
| hCl1a AAA-RLI DANA | 63.0 |
| hCl1a AAA-RLI DANA afuc | 64.9 |
| hCl1a TL-RLI DANA | 68.0 |
| hCl1a IE-RLI DANA | 58.3 |

TABLE 22
N-glycan analysis

| Immunocytokine or | Man5 species | | Sialic acid species | |
|---|---|---|---|---|
| antibody | CHOKI | FUT8KO | CHOKI | FUT8KO |
| hCl1a | 7.8% | Not tested | 0 | Not tested |
| hCl1a-RLI DANA | 10.1% | 5.4% | 0 | 0 |
| hCl1a DLE-RLI DANA | 1.6% | 0 | 0 | 0 |
| hCl1a DE-RLI DANA | 2.4% | 0 | 0 | 0 |
| hCl1a AAA-RLI DANA | 19.2% | 7.5% | 0 | 0 |
| hCl1a TL-RLI DANA | 16.4% | Not tested | 23.0% | Not tested |
| hCl1a IE-RLI DANA | 21.6% + 4% | Not tested | 0 | Not tested |
| | Man6 | | | |

### 18. Claudin18.2 Immunocytokine Mouse In Vivo Efficacy Studies

The purpose of this study is to test the in vivo therapeutic efficacy of hCl1a-RLI immunocytokines in a mouse model. Female NMRI nude mice are implanted at 5-7 weeks of age with pancreatic human cell line derived xenograft BXPC3 (ATCC CRL-1687™) exogenously expressing Claudin18.2 (BXPC3-CLDN18.2). Tumours are implanted by unilateral subcutaneous injection. The animals ae randomized based on the tumour volume around 100 mm³. Mice are allocated to different groups (n=7 per group) and treated according to the Table 23 at day 1. The animals are checked twice weekly for weight loss and tumour volume. Tumour volume is measured by calliper and is expressed in mm³ using the formula: “V=(L×W×W)/2, where V is tumour volume, L is tumour length (the longest tumour dimension) and W is tumour width (the longest tumour dimension perpendicular to L). Mice are euthanized reaching a tumour burden of 2000 mm³ or experiencing significant body weight loss (overall more than 30%, or more than 20% in two consecutive days).

TABLE 23
mouse treatment regimen (“afuc” for afucosylated)

| | | Total daily | | | mice/ |
|---|---|---|---|---|---|
| Group | Test item | dose | Schedule | Route | group |
| 1 | Vehicle (0.9% NaCl) | 5 mg/kg | D1 | i.v. | 7 |
| 2 | hCl1a-RLI NAx1 | 5 mg/kg | D1 | i.v. | 7 |
| 3 | hCl1a-RLI NAx1 afuc | 5 mg/kg | D1 | i.v. | 7 |
| 4 | hCl1a | 5 mg/kg | D1 | i.v. | 7 |
| 5 | Zolbetuximab | 5 mg/kg | D1 | i.v. | 7 |

### 19. Anti-PD-1 Antibody and SOT201 Synergize in Activation of CD8+ T Cells

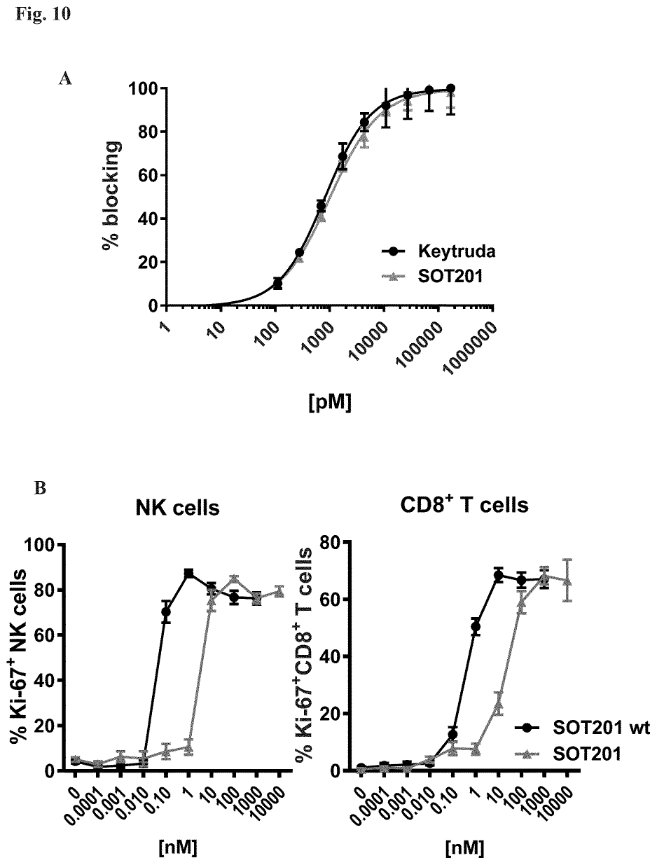

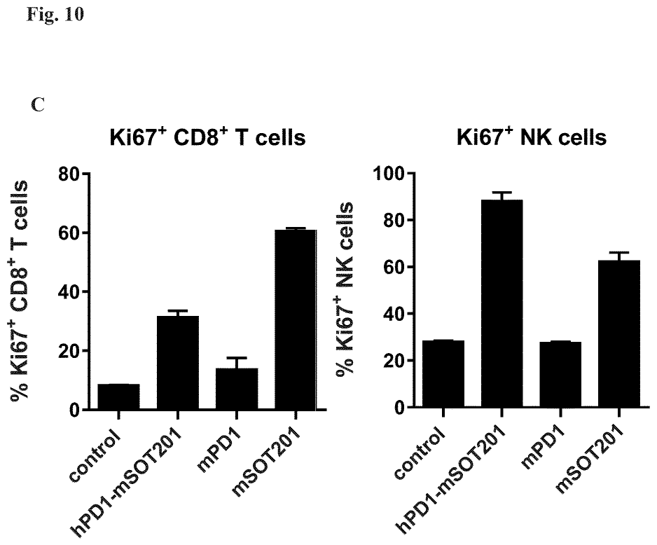

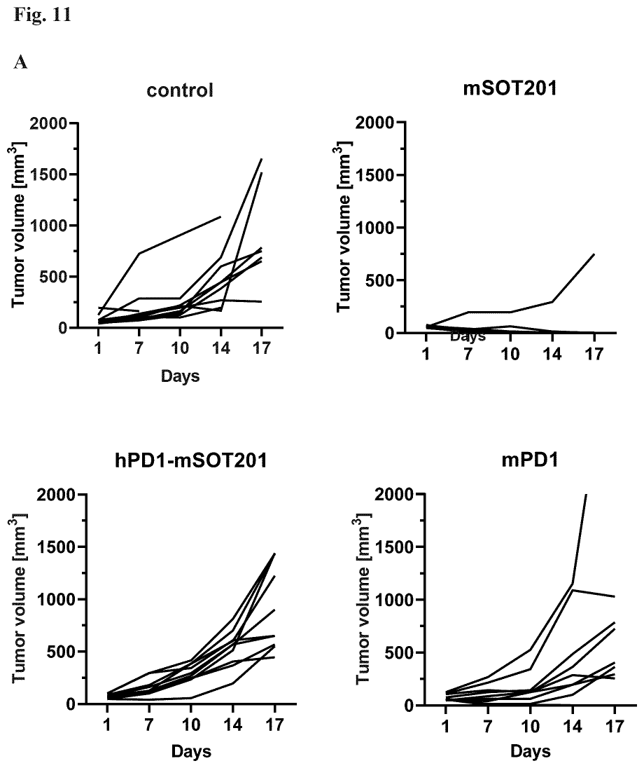

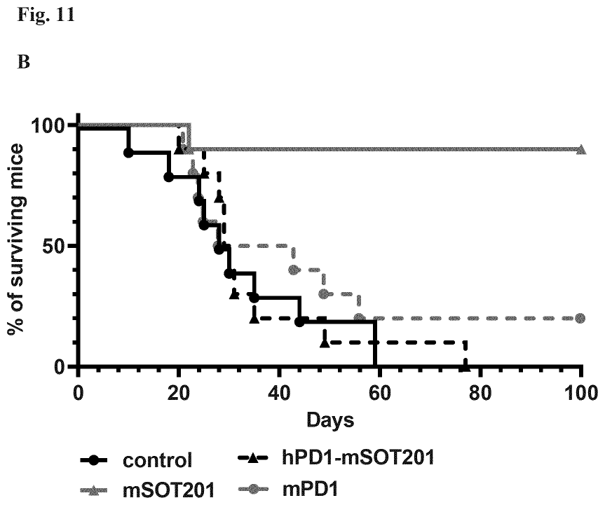

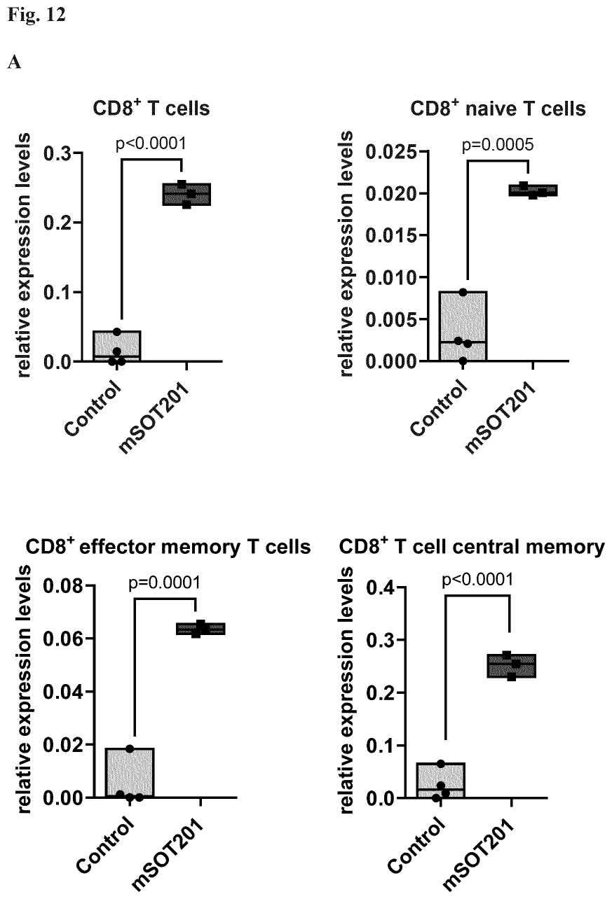

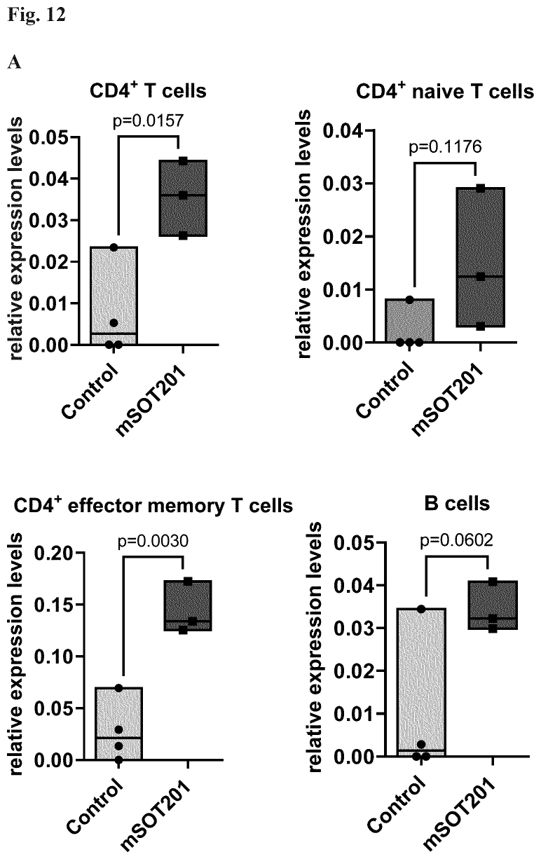

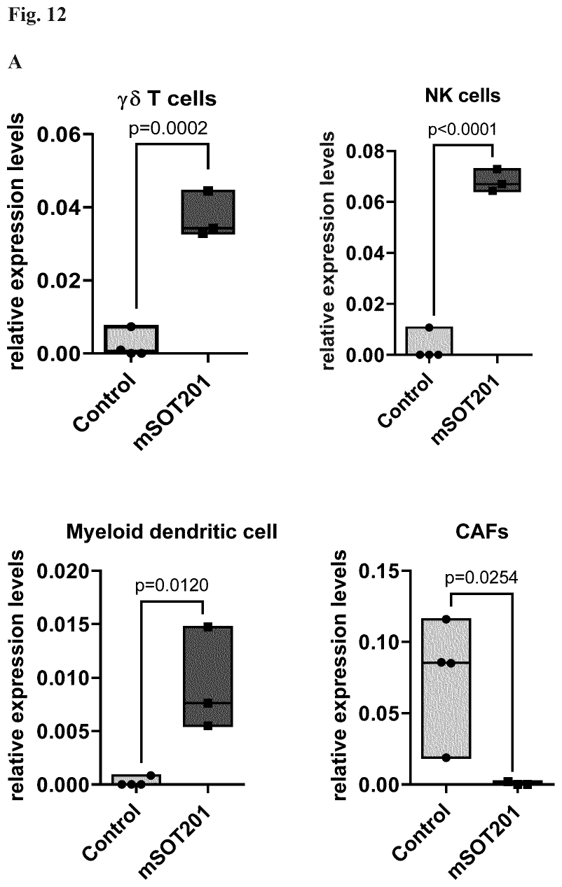

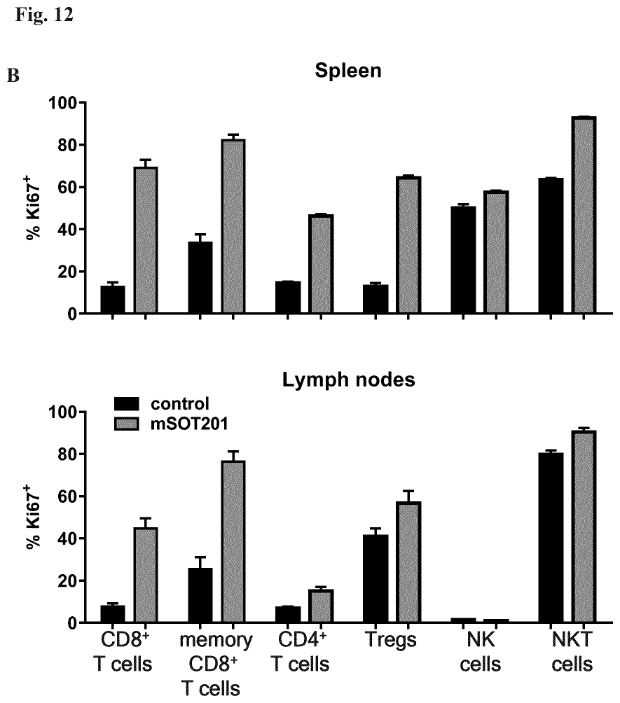

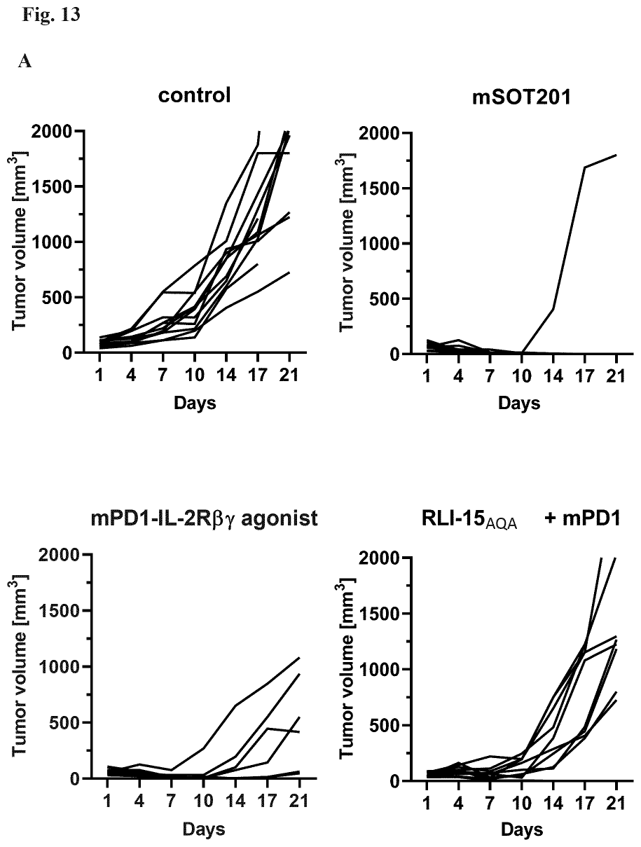

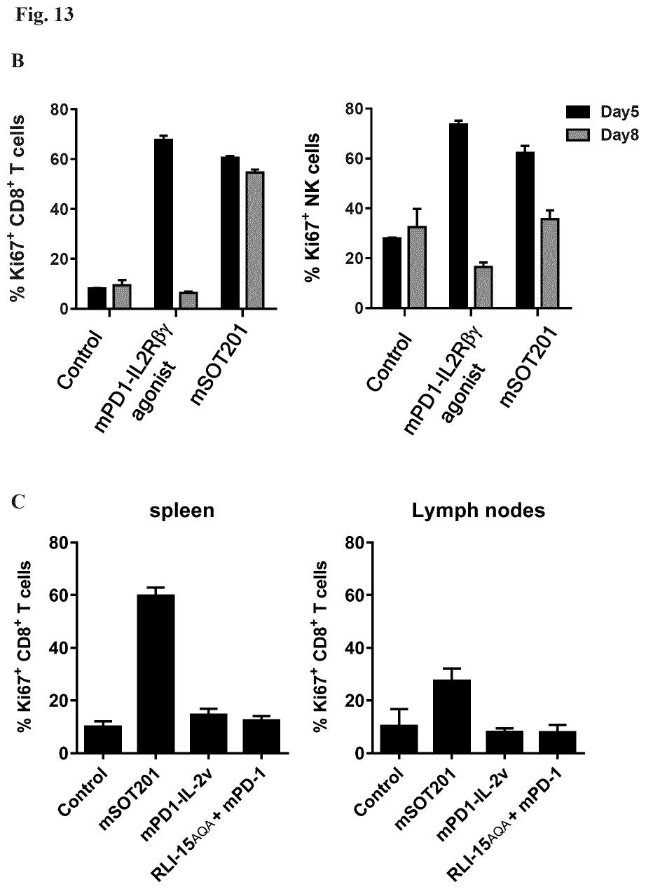

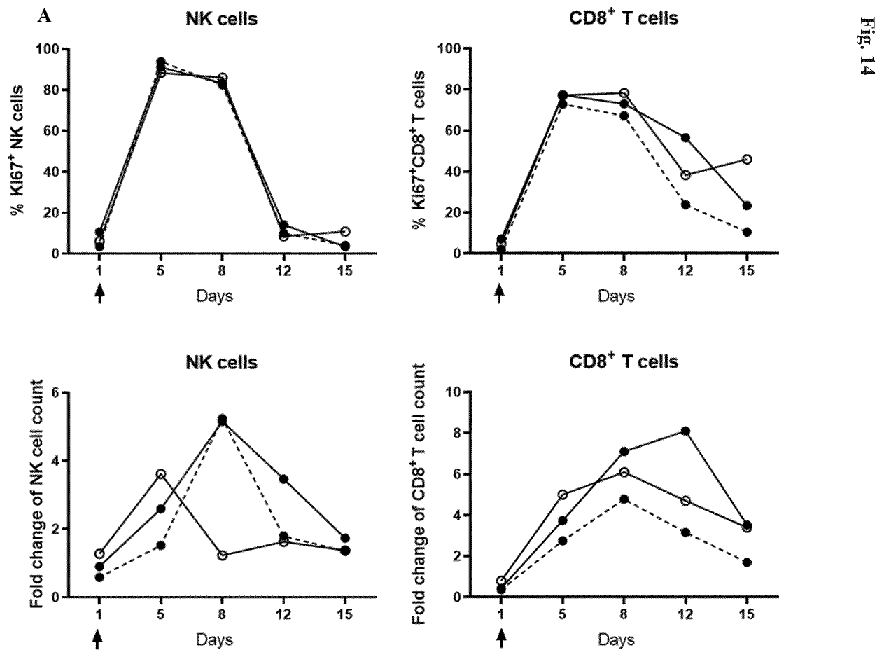

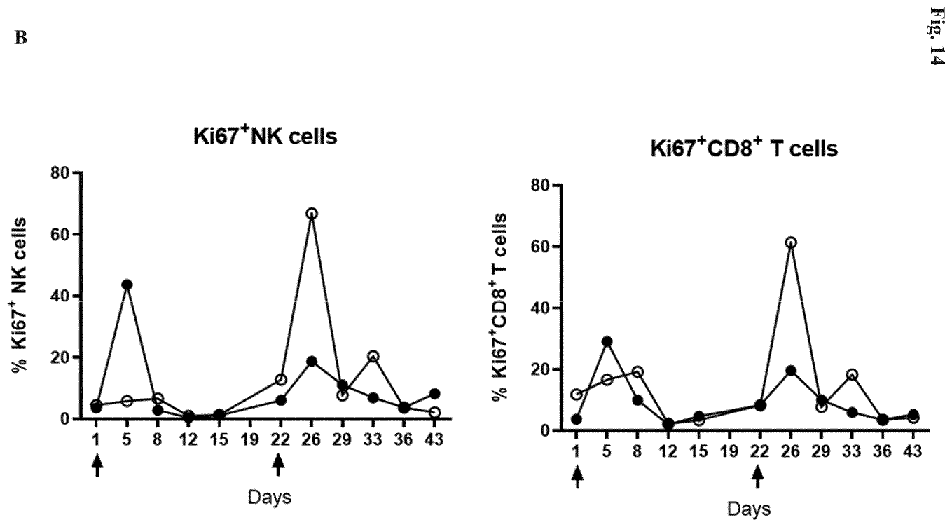

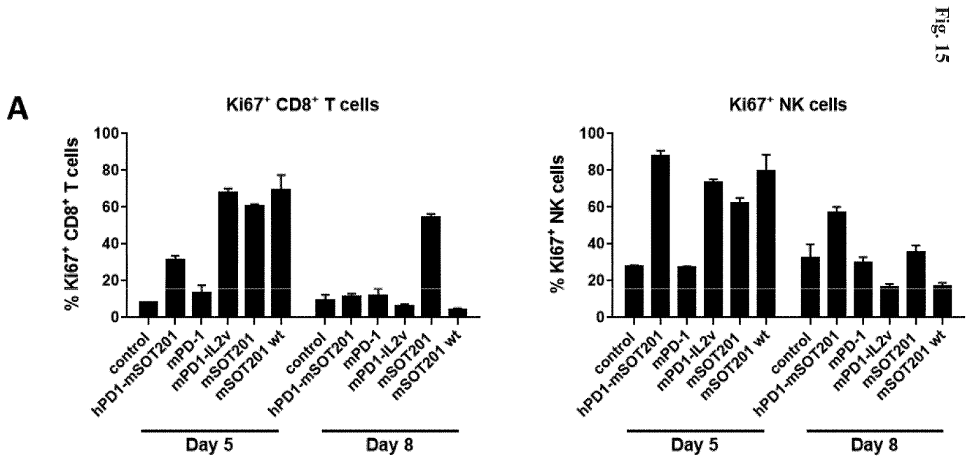

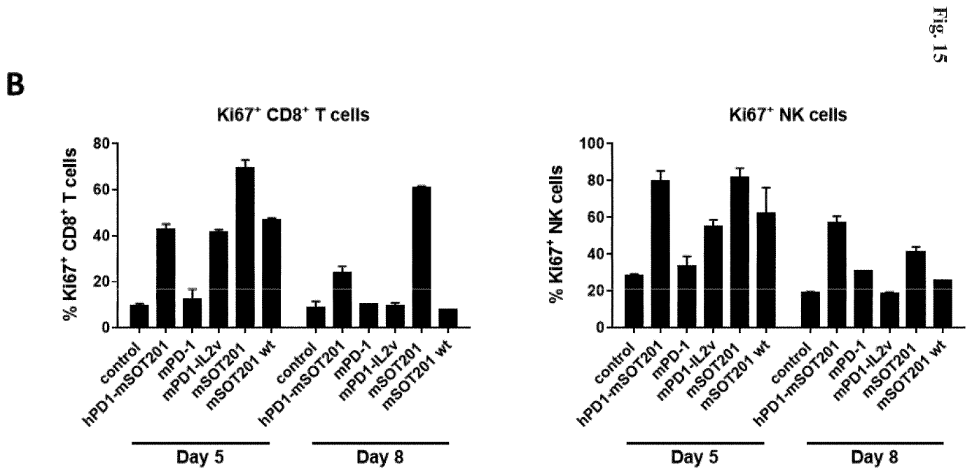

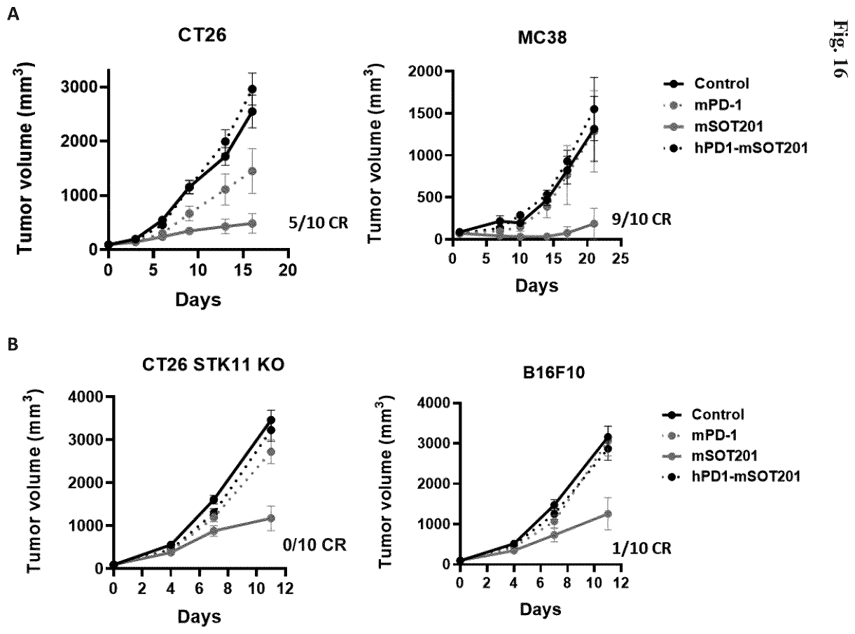

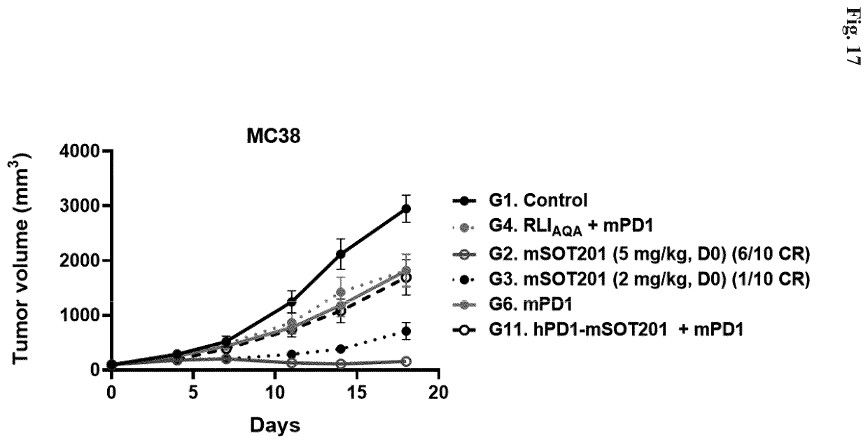

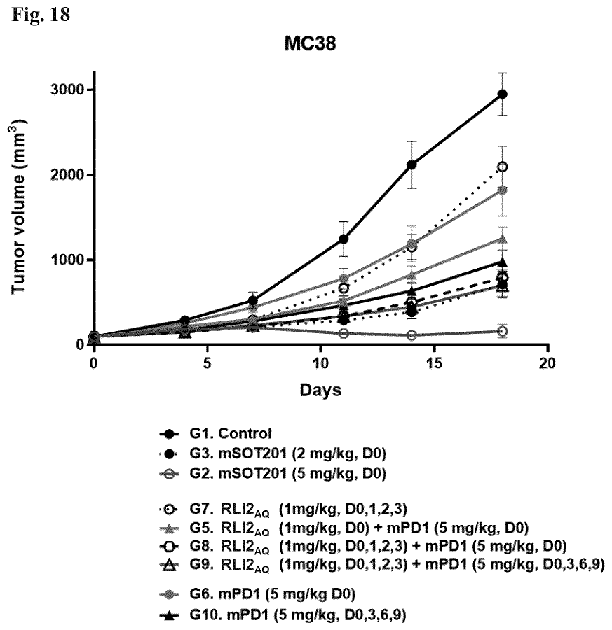

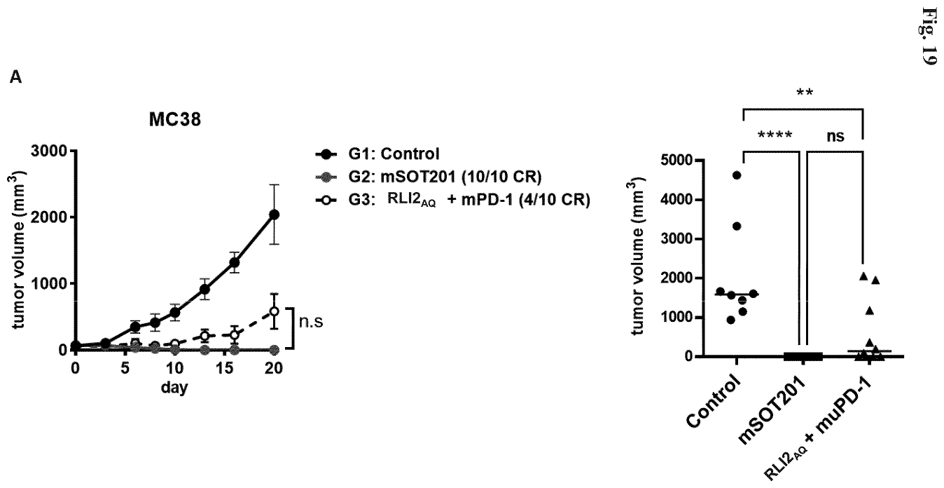

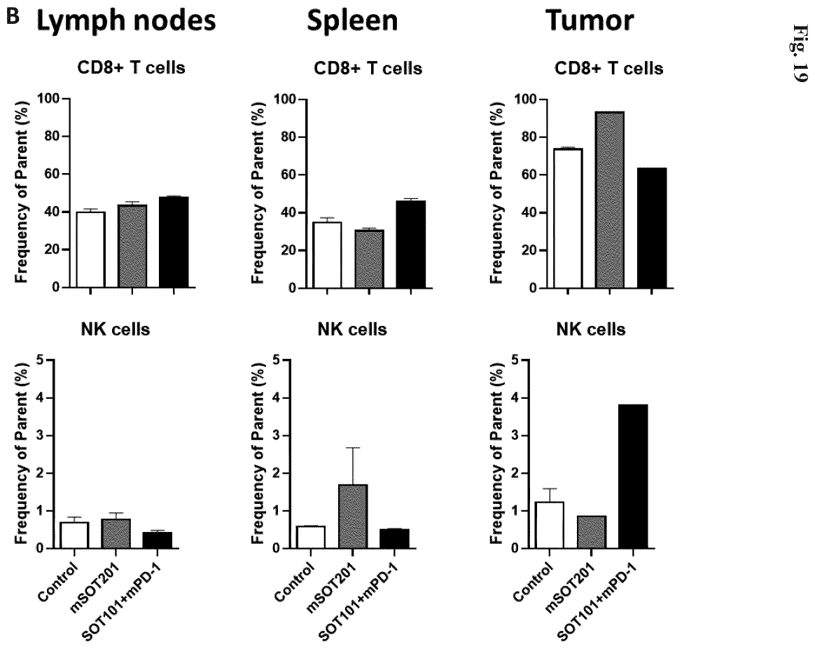

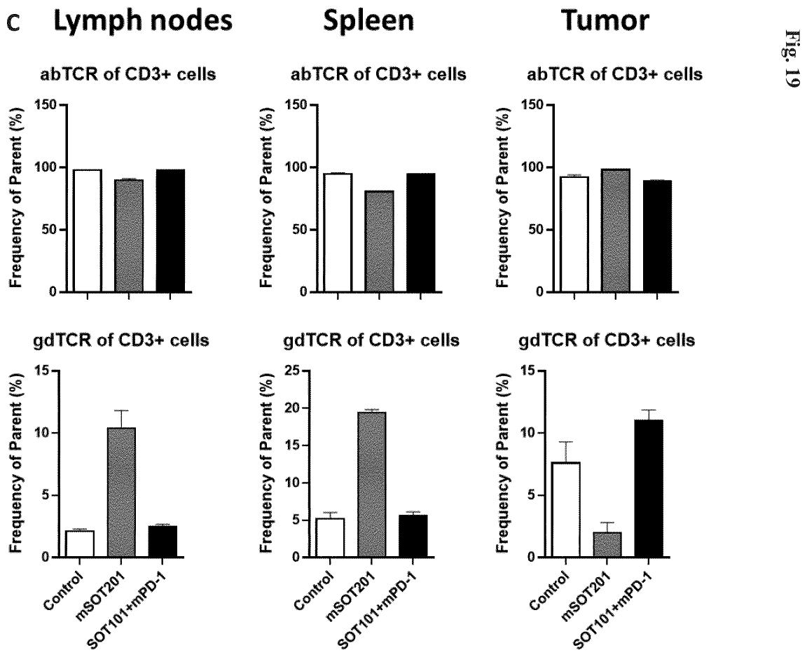

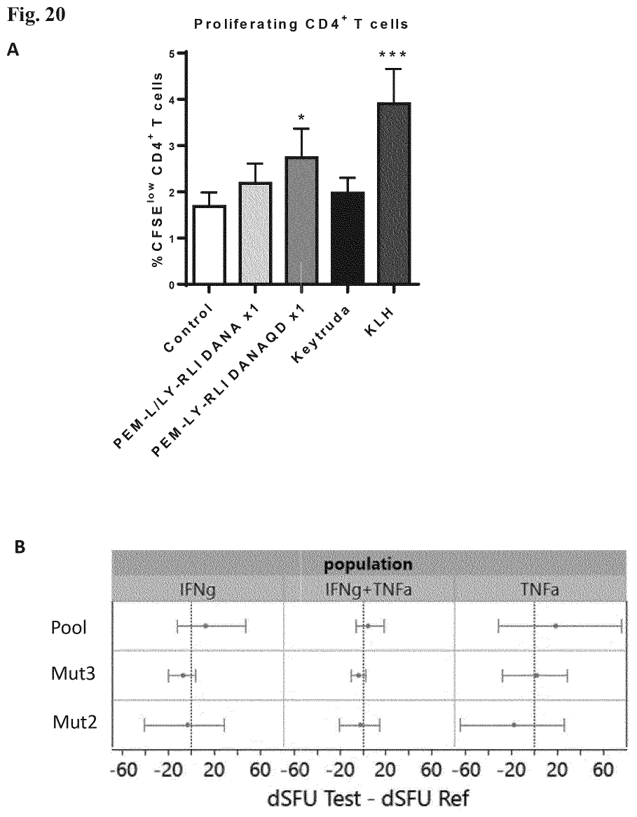

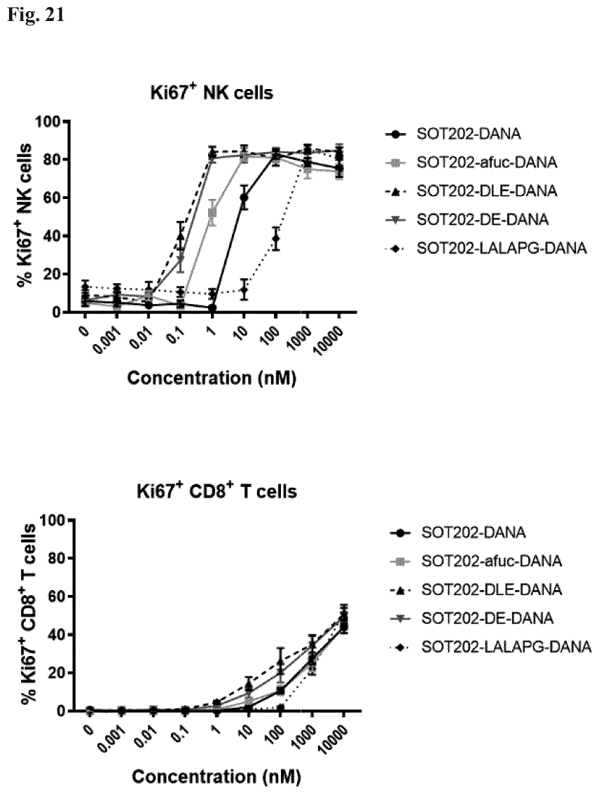

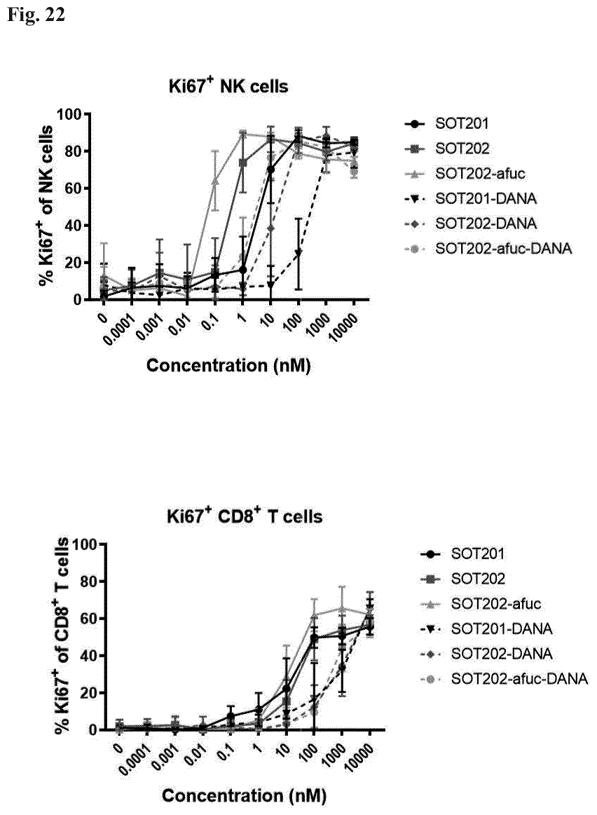

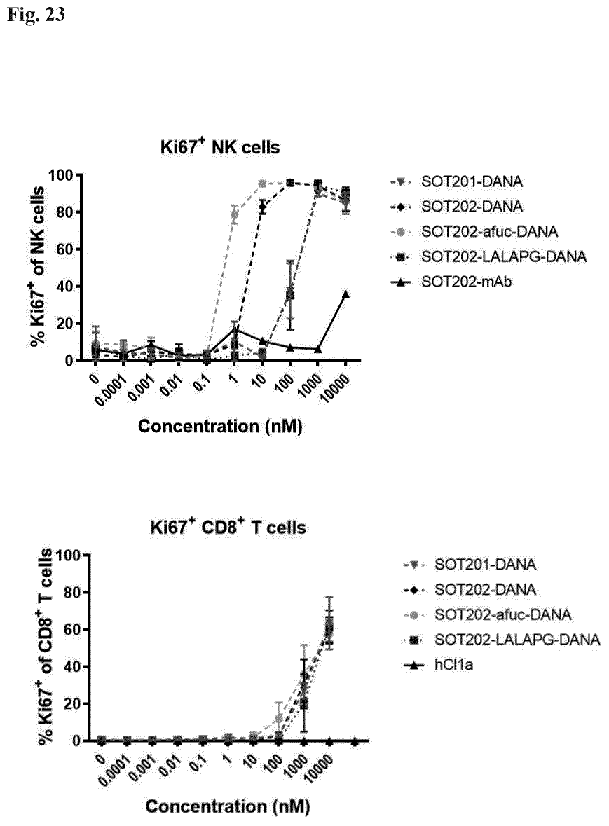

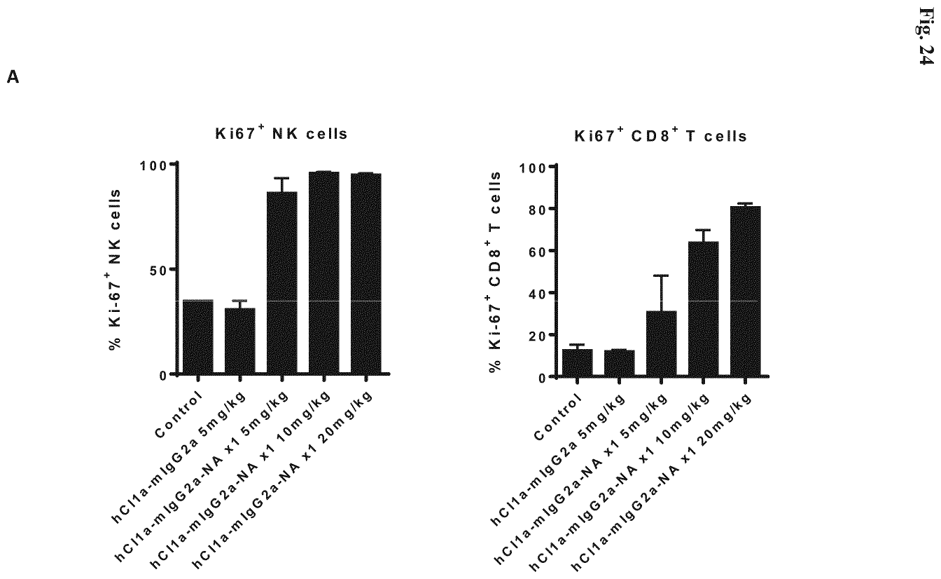

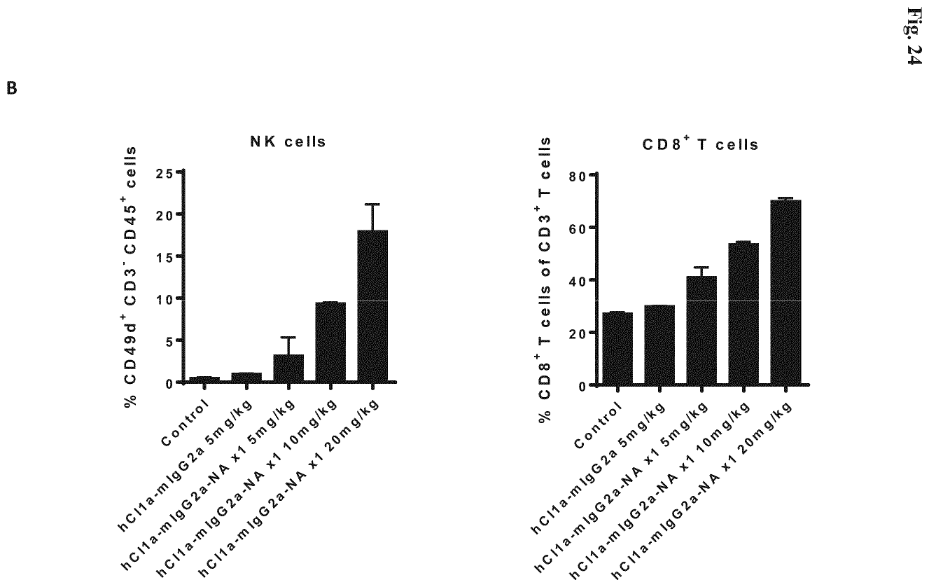

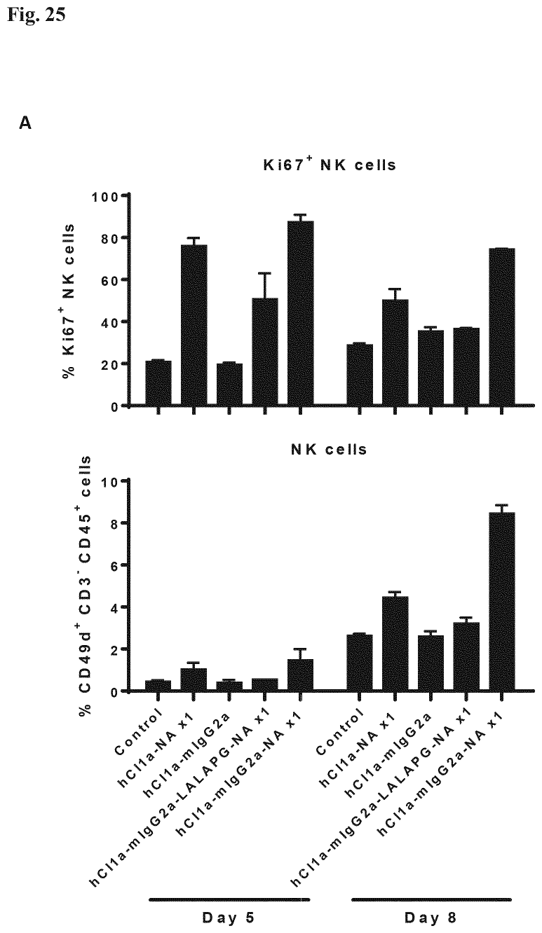

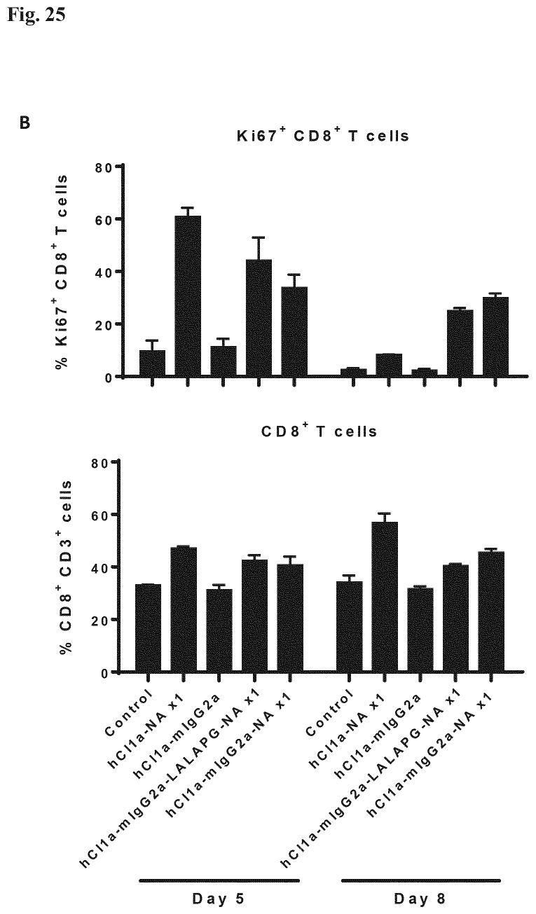

SOT201 is a heterodimeric immunocytokine with an antibody derived from the humanized IgG 4 pembrolizumab with T366W—knob/T366S, L368A, Y407V-hole substitutions, L235E substitution, and deleted terminal K of the heavy chains, fused to RLI-15_{AQA} at the C-terminus of the knob heavy chain, see SEQ ID NO: 22, SEQ ID NO: 38, SEQ ID NO: 24). SOT201 and Keytruda (pembrolizumab) were compared in the PD-1/PD-L1 blockade assay according to Example 1. FIG. 10A shows that SOT201 effectively blocks PD-1/PD-L1 interactions similarly to the anti-PD-1 antibody Keytruda. Determined K_{D} values for SOT201 and pembrolizumab are shown in Table 24.

TABLE 24
K_{D} values for SOT201 and pembrolizumab at 4° C. and 37° C.

| | |
|---|---|
| 4° C. binding | 37° C. binding |
| K_{D} [nM] | K_{D} [nM] |

| | 1 h | 4 h | 1 h | 4 h |
|---|---|---|---|---|
| SOT201 | 0.28 | 0.26 | 0.23 | 0.21 |
| Keytruda R | 0.23 | 0.23 | 0.22 | 0.21 |
| (pembrolizumab) | | | | |

Human PBMC from 11 healthy donors were stimulated for 7 days in vitro with SOT201 having the RLI2AQ N65A (RLI-15_{AQA}) variant or with a control molecule having identical antibody heavy and light chains as SOT201 but with the RLI2_{AQ} variant without a reduced binding of the IL-15 moiety to the IL-2/IL-15Rβγ (“SOT201 wt”). Cell proliferation was determined by measuring Ki-67⁺ NK cells and CD8⁺ T cells by flow cytometry analysis. SOT201 activates proliferation of NK and CD8⁺ T cells at higher EC50 concentration in comparison to the comparable immunocytokine molecule with an RLI-15 molecule without reduced receptor binding (SOT201 wt) (FIG. 10B).

A murine surrogate SOT201 (mSOT201, see SEQ ID NO: 39, SEQ ID NO: 40 and SEQ ID NO: 41) comprising the anti-murine PD-1 antibody RMP1-14 (BioXCell, Lebanon, NH, USA) with analogous substitutions for heterodimerization (E356K, N399K/K409E, K439D), ADCC silencing (D265A) and stabilization (dK) fused to RLI-15_{AQA} was compared to single activity controls represented by the monoclonal anti-murine PD-1 antibody RMP1-14 as such (mPD1) and the anti-human PD1 mouse IgG1-RLI-15_{AQA} (hPD1-mSOT201), which does not exert any PD-1 blocking activity in the C57BL/6 mouse, as an RLI-15_{AQA} control with a similar in vivo half-life as mSOT201. Cell proliferation (Ki67) was detected in spleen by flow cytometry 5 days after IV injection of compounds at equimolar amount to 5 mg/kg of mSOT201 in healthy C57BL/6 mice (n=2/group). The anti-PD-1 antibody and the RLI-15_{AQA} mutein moieties in the murine surrogate mSOT201 showed a synergistic effect on CD8⁺ T cell proliferation (FIG. 10C).

### 20. Tumor Regression in MC38 Mouse Model

C57BL/6 mice (hPD1-transgenic) were implanted with syngeneic MC38 cell line. Test agents mSOT201, hPD1-mSOT201 and mPD1 were injected IV on day 1 (randomization day, tumor volumes 80-100 mm³) (n=10/group) at equimolar amounts to 5 mg/kg mSOT201 and compared to control (NaCl). mSOT201 induced tumor regression in 9 out of 10 mice after a single IV administration, whereas in comparison the monoclonal anti-mouse PD-1 antibody (mPD1) and the anti-human PD-1 mouse IgG1-RLI-15 mutein immunocytokine (hPD1-mSOT201) exerting no anti-PD-1 effect in mice only showed minor effects on tumor growth compared to the control mice (FIG. 11A). Similarly, the synergistic activity of the anti-murinePD-1 antibody and the RLI-15_{AQA} mutein in the fusion protein (mSOT201) compared to the anti-mousePD-1 antibody alone (mPD1) or the anti-humanPD1 mouse IgG1-RLI-15 mutein immunocytokine (hPD1-mSOT201) as a control for the RLI-15_{AQA} mutein alone is shown in the surviving mice in the time course up to 100 days post treatment (FIG. 11B).

### 21. Induction of Pathways and Genes Connected to Anti-Tumor Immunity in MC38 Tumors and Activation of Immune Cells in Spleen and Lymph Nodes

RNA isolation: RNA samples were isolated from tumors of syngeneic MC38 tumor bearing C57BL/6 mice 7 days after a single IV administration of mSOT201 (5 mg/kg). 3 mice were treated with mSOT201 (5 mg/kg) IV on day 1 (randomization day, tumor volumes 80-100 mm3), 4 control mice were left untreated. RNA was isolated from tumour tissue by using RNeasy MicroKit. The quality of RNA samples was checked using the Agilent Bioanalyzer RNA Nano Chip and the Qubit HS RNA assay.

RNA seq analysis: The sequencing libraries were prepared from RNA samples by the SMARTer® Stranded Total RNA-Seq Kit v3—Pico Input Mammalian Kit (Takara Bio USA, Inc.), library quality control was performed employing the capillary gel electrophoresis system (Agilent Bioanalyzer with the HS DNA chip) and the Qubit HS DNA Assay, and sequencing was done on NovaSeq 6000 using the NovaSeq 6000 300 cycles Reagent Kit in 2×151 bp run.

Data analysis: Raw data were processed according to the standard RNA-seq pipeline including the following steps: quality control (via FastQC and FastqScreen), adapter trimming (trimmed 8 bp in Read2 by using seqtk), mapping to the reference genome GRCm39 (using HISAT2) and transcript counting (with ht-seq). The obtained output, quantification files containing the number of transcripts for each sample, were further processed via R packages and ggplot2, tydiverse, dplyr. Raw counts were normalized via DESeq2 median of ration normalization. Differential gene expression analysis was performed using DESeq2 (version 1.24.0) in R (abs(log 2FC)=1, FDR<0.05). Heatmaps were created using ComplexHeatmap package in R. Functional and enrichment analysis of DEGs was performed using the ClusterProfiler and the web-based tool Gene ontology (GO). To calculate TPM values for cell population analysis, salmon tool was used on trimmed fastq files. Analysis of cell population was performed by TIMER 2.0 and xCell tools.

Results: Differential expression analysis (abs(log 2FC)=1, FDR<0.05) resulted in upregulation of 800 mouse genes and downregulation of 1910 mouse genes in mSOT201-treated tumors compared to control samples. Enrichment analysis of gene Ontology (GO) terms mainly identified upregulated DEGs linked to activation of ap T cells, γδ T cells, B cells, NK cells, cytotoxicity, cell killing, cytokine production, cell chemotaxis and cell adhesion while downregulated genes were linked to tumor development and tumor signaling. These data indicate that mSOT201 activates both innate as well as adaptive immunity in the tumor microenvironment. Next, we employed “metagene” markers to estimate the relative abundance of different immune cell populations in the tumor microenvironment. In line with the whole-transcriptome findings, mSOT201-treated samples were enriched for gene sets associated with CD8⁺ T cells (p<0.001), CD8⁺ naïve T cells (p<0.0005), CD8+effector memory T cells (p=0.001), CD8⁺ T cell central memory (p<0.001), γδ T cells (p=0.0002), NK cells (p<0.001), CD4⁺ T cells (p=0.0157), CD4⁺ naïve T cells (p=0.1176), CD4+effector memory T cells (p=0.003), B cells (p=0.0602), myeloid dendritic cells (p=0.0120). On the other hand, the gene sets associated with cancer-associated fibroblast was markedly reduced (p=0.0254) (FIG. 12A).

mSOT201 induced proliferation of selected immune cell populations in spleen and lymph nodes in MC38 tumor bearing mice (FIG. 12B). Cell proliferation (Ki67) was detected by flow cytometry on day 7 after the mSOT201 treatment of the established tumors (80-100 mm³) (n=2).

### 22. EC50 Values of Different IL2/IL-15Rβγ Agonists on Kit225 Cells

EC50 values of RLI-15 (SOT101), SOT201 (PEM-RLI-15_{AQA}), hPD-1-IL-2v and αhPD1-IL-15m M1 were determined as described in Example 1. In hPD-1-IL-2v one IL-2 mutein IL-2v (SEQ ID NO: 43) is fused to the C-terminus of one heavy chain of an anti-humanPD-1 antibody as described in WO 2018/184964a1 (with sequences of Seq id no.: 22, 23 and 25 therein). In αhPD1-IL-15m M1 one IL-15 mutein with the mutations N1A-D30N-E46G-V49R (SEQ ID NO: 44) is fused to the C-terminus of one heavy chain of an anti-humanPD-1 antibody as described in WO 2019/166946a1 (see FIG. 1D therein, SEQ ID NO: 89, 74 and 65 therein). EC50 values are show in Table 25.

TABLE 25
EC50 of selected IL-2/IL-15Rβγ agonists on kit225 cells

| | EC50 [pM] |
|---|---|
| SOT101 (RLI-15) | 35 |
| SOT201 (PEM-RLI-15_{AQA}) | 15160 |
| PD1-IL-2v | 3018 |
| αhPD1-IL-15m M1 | 3307 |

A further interesting candidate to be tested is the αhPD1-IL-15m M2 with on IL-15 mutein with mutations N1G-D30N-E46G-V49R-E64Q (SEQ ID NO: 45) is fused to the C-terminus of one heavy chain of an anti-humanPD-1 antibody as described in WO 2019/166946a1 (see FIG. 1C therein, Seq id no: 90, 74 and 65 therein).

Accordingly, SOT201 has a substantially lower EC50 on kit225 cells than PD1-IL-2v and αhPD1-IL-15m M1, expected to allow for higher dosing and longer half-life in vivo to exert also a stronger and longer lasting effect with respect to the activity disrupting the anti-PD-1/PD-L1 interaction.

23. Comparison of mSOT201 with mPD1-IL-2Rβγ Agonist in the MC38 Tumor Model

mSOT201 (mouse SOT201 surrogate) was compared to control (NaCl), the anti-murinePD-1 antibody RMP1-14 fused to the IL-2v IL-2 mutein (mPD1-IL-2Rβγ agonist) and the combination of the RLI-15_{AQA} and the mPD1 antibody in the MC38 tumor model in a single IV administration as described in Example 20. The dosing of mPD1-IL-2Rβγ was selected to match the NK and CD8⁺ T cell proliferation on day 5 of 5 mg/kg of mSOT201 after IV administration in healthy C57/BL6 mice, resulting in an equivalent dose of 0.25 mg/kg mPD1-IL-2Rβγ. Cell proliferation (Ki67+) was detected by flow cytometry. mSOT201 induced activation of CD8⁺ T cells and NK cells which persisted up to day 8 in contrast to the mPD1-IL-2Rβγ agonist (FIG. 13B).

mPD1-IL-2Rβγ is an IL-2/IL-15Rβγ agonist where the IL-2 mutein IL-2v (SEQ ID NO: 43) comprises the substitutions F42A, Y45A and L72G relative to the IL-2 sequence reducing the affinity to the IL-2Rα (see WO 2018/184964A1, e.g., bridging para. of pages 27 and 28) and the further substitutions T3A to eliminate O-glycosylation at position 3 (bridging para. of pages 28 and 29) and C125A to increase expression or stability (page 30, 3^{rd} para.).

The murine surrogate of SOT201 (mSOT201) induced tumor regression in 9 out of 10 MC38 tumor-bearing mice after a single IV administration comparing to 5 out of 10 for the mPD1-IL-2Rβγ agonist, whereas the combination of the RLI-15_{AQA} with the mPD1 antibody only led to a delay of tumor growth compared to the control mice (FIG. 13A).

mSOT201 induced proliferation of NK and CD8⁺ T cells in MC38 tumor bearing mice which persisted 7 days after dosing in contrast to the mPD1-IL-2Rβγ agonist and the equimolar amount of RLI-15_{AQA} in combination with mPD 1. The treatment of MC38 tumors was at randomization day 1, tumor volumes 100 mm³ (n=10/group).

Further, mSOT201_induced a strikingly longer activation of CD8⁺ T cells and NK cells still persisting at day 8 in contrast to mPD1-IL-2Rβγ agonist, which showed marked reductions of proliferating cells at day 8 (FIG. 13B).

SOT201 also induced proliferation of NK and CD8⁺ T cells in spleen and lymph nodes of MC38 tumor bearing mice which persisted 7 days after dosing in contrast to mPD1-IL-2v and the equimolar amount of the combination of RLI-15_{AQA} and the mPD1 antibody (FIG. 13C).

### 24. PK Profile of SOT201 in Cynomolgus Monkeys

SOT201 was administered IV at 0.6 mg/kg on day 1 to cynomolgus monkeys and proliferation (Ki67+) and absolute cell numbers of NK and CD8⁺ T cells were determined over time by flow cytometry and haematology. SOT201 induced high proliferation and expansion of NK (˜90% at day 5) and CD8⁺ T cells (about 80% at day 5) in blood of cynomolgus monkeys after an IV administration (FIG. 14A). Pharmacokinetic parameters are shown in Table 26.

TABLE 26
Pharmacokinetic parameters of SOT201 in cynomolgus monkeys

| | Dose | No. and sex | AUCₗₐₛₜ | Cₘₐₓ | Tₘₐₓ | T_{1/2} |
|---|---|---|---|---|---|---|
| Day | [mg/kg] | of animals | [ng · h/ml] | [ng/ml] | [h] | [h] |
| 1 | 0.6 | 3F | 456,751.96 | 14,633.52 | 1 | 17.63 |

SOT201 induced activation of NK and CD8⁺ T cells after a repetitive IV administration in cynomolgus monkeys (FIG. 14B)

### 25. PD Activity of Mouse SOT201 Surrogates

The first aim of the study was to evaluate whether the treatment with mouse surrogate molecule mSOT201 (see Example 19) has an additive/synergistic effect on the CD8⁺ T cell proliferation, when compared to the treatment with hPD1-mSOT201 or mPD-1 in C57BL/6 mice. The second aim of the study was to compare the pharmacodynamic activity of mSOT201 wt mouse surrogate molecule with a mouse surrogate molecule mPD1-IL2v in C57BL/6 mice. The description of tested mouse surrogate molecules is described in Table 27. PD activity was evaluated on day 5 and day 8. FACS analysis was performed as described above.

TABLE 27
Description of the mouse surrogate molecules

| Molecule | Molecule characteristics |
|---|---|
| mSOT201 | Mouse anti-PD-1 antibody (clone RMP1-14) mouse IgG1-D265A |
| | RLI-15_{AQA} genetically fused to anti-PD-1 antibody |
| hPD1-mSOT201 | Human anti-PD-1 antibody (pembrolizumab) mouse IgG1-D265A |
| | RLI-15AQA genetically fused to anti-PD-1 antibody |
| mPD-1 | Mouse anti-PD-1 antibody (clone RMP1-14) mouse IgG1-D265A |
| mSOT201 wt | Mouse anti-PD-1 antibody (clone RMP1-14) mouse IgG1-D265A |
| | RLI2 genetically fused to anti-PD-1 antibody |
| mPD1-IL2v | Mouse anti-PD-1 antibody (clone RMP1-14) mouse IgG1-D265A |
| | IL2v: IL-2 mutein with T3A F42A Y45A L72G C125A substitutions |
| | genetically fused to anti-PD-1 antibody (see SEQ ID NO: 51, SEQ ID NO: 52, |
| | SEQ ID NO: 53) |

TABLE 28
Potency of mouse surrogates in comparison
to human molecules in kit225 assay

| | EC50 | Relative potency to |
|---|---|---|
| | kit225 cells [pM] | SOT101 |
| SOT101 | 35.9 | — |
| SOT201 | 9998 | 0.36% |
| hPD1-mSOT201 | 19983 | 0.18% |
| mSOT201 | 26786 | 0.13% |
| SOT201 wt (with RLI2_{AQ}) | 248.8 | 14.43% |
| mSOT201 wt (with RLI2_{AQ}) | 558.4 | 6.42% |
| IL-2 | 672.9 | 5.33% |
| mPD1-IL2v | 3082 | 1.16% |
| PD1-IL2v | 3018 | 1.19% |

As pembrolizumab does not recognize the murine PD-1, the hPD-1-mSOT201 represents a control for an RLI-15_{AQA} bound to a non-binding antibody with a similar PK profile and therefore reflects the PD activity of the RLI-15_{AQA} Molecule with such PK profile. The mPD-1 molecule reflects the PD activity of the anti-PD-1 antibody alone. With respect to the activation of CD8⁺ T cells, mSOT201 shows a more than additive effect (i.e. synergistic) compared to its single component surrogates hPD1-mSOT201 and mPD-1 at Day 5 and even more at Day 8 dosed at equimolar amounts. In comparison, both mPD1-IL2v and mSOT201 wt (both having a more active IL-2/IL-15Rβγ agonist), dosed lower given their expected high activity at Day 5, show a bit higher activation of CD8⁺ T cells on Day 5, but such effect is only short lasting, as at Day 8 activation of CD8⁺ T cells is much stronger for mSOT201. Looking at activated NK cells, differences are not so pronounced. As expected, mPD-1 does not activate NK cells, whereas hPD1-mSOT201, mPD1-IL2v, mSOT201 and mSOT201 wt strongly activate at Day 5, with mSOT201 somewhat weaker than the others. At Day 8, again mSOT201 exhibits a stronger activation of NK cells compared to mPD1-IL2v and mSOT201 wt. (FIG. 15 A)

A similar picture was observed, when to mSOT201, hPD1-mSOT201 and mPD-1 were dosed at double the amounts of A, whereas mSOT201 wt and mPD1-IL2v were dosed at lower amounts (see FIG. 15 B), as they likely already had reached maximal activation of cells in experiment A. As expected, mSOT201 wt and mPD1-IL2v showed a reduced activation of both CD8⁺ T cells and NK cells, which again was down at control level at Day 8 for CD8⁺ T cells.

These data show, that SOT201 having a marked reduced binding to IL-2/IL-15Rβγ together with its anti-PD-1 moiety is both a strong and long-lasting activator of NK and CD8⁺ T cells, whereas molecules with higher IL-2/IL-15Rβγ agonistic activity show a much shorter activation especially of CD8⁺ T cells. It is hypothesized that the avidity effect of simultaneously binding PD-1 and the IL-2/IL-15Rβγ of PD-1 expressing CD8⁺ T cells in cis (i.e., on the same CD8⁺ T cell) or in trans (i.e., between different CD8⁺ T cells in close proximity) leads to such preferential activation of CD8⁺ T cells.

26. Anti-Tumor Efficacy Activity of mSOT201 in PD-1 Sensitive and PD-1 Treatment Resistant Mouse Models

The aim of the study was to evaluate the anti-tumor activity of mSOT201 in anti-PD-1 treatment sensitive (CT26, MC38) and in anti-PD-1 treatment resistant (B16F10, CT26 STK11 ko) mouse models. The description of tested mouse surrogate molecules is described in Table 29.

TABLE 29
Description of the mouse surrogate molecules

| Molecule | Molecule characteristics |
|---|---|
| mSOT201 | Mouse anti-PD-1 antibody (clone RMP1-14) mouse IgG1-D265A |
| | RLI-15_{AQA} genetically fused to anti-PD-1 antibody |
| hPD1-mSOT201 | Human anti-PD-1 antibody (pembrolizumab) mouse IgG1-D265A |
| | RLI-15_{AQA} genetically fused to anti-PD-1 antibody |
| mPD-1 | Mouse anti-PD-1 antibody (clone RMP1-14) mouse IgG1-D265A |

The murine surrogate molecule of SOT201-mSOT201—as compared to its single component surrogates mPD-1 and hPD1-mSOT201 shows a synergistic effect in the tested PD-1 sensitive tumor models CT26 and MC38 with 5 out of 10 and 9 out of 10 complete responses. (FIG. 16 A)

Even in tumor models known to be resistant to anti-PD-1 therapy, mSOT201 showed a synergistic effect compared to its single components, although the therapeutic effect was not as strong as for the sensitive models showing only 1 complete response out of 10 mice for the B16F10 model. (FIG. 16 B)

27. Anti-Tumor Efficacy Activity of mSOT201 vs RLI-15_{AQA} Mutein+Anti-PD-1 Antibody

The aim of the study was to evaluate the anti-tumor activity of mSOT201 vs. RLI-15 AQA mutein+anti-PD-1 treatment in MC38 mouse models. The description of tested mouse surrogate molecules is described in Table 30.

TABLE 30
Description of the mouse surrogate molecules

| Molecule | Molecule characteristics |
|---|---|
| mSOT201 | Mouse anti-PD-1 antibody (clone RMP1-14) mouse IgG1-D265A |
| | RLI-15_{AQA} genetically fused to anti-PD-1 antibody |
| hPD1-mSOT201 | Human anti-PD-1 antibody (pembrolizumab) mouse IgG1-D265A |
| | RLI-15_{AQA} genetically fused to anti-PD-1 antibody |
| RLI-15 AQA | RLI-15 mutein |
| mPD-1 | Mouse anti-PD-1 antibody (clone RMP1-14) mouse IgG1-D265A |

The fusion of the anti-PD-1 moiety with the IL-2/IL-15βγ agonist RLI-15_{AQA} (at two doses, G2 and G3) showed a strong synergistic effect compared to the combination of the individual equimolar components (G4: RLI-15_{AQA}+mPD1, or G11: hPD1-mSOT201+mPD1), see FIG. 17. It is hypothesized that the temporal and spatial linkage of activation of PD-1 positive immune cells is mechanistically stronger than the activation of immune cells by the individual components.

28. Anti-Tumor Efficacy Activity of mSOT201 vs SOT101+Anti-PD-1 Antibody

The aim of the study was to evaluate the anti-tumor activity of mSOT201 vs SOT101+anti-PD-1 treatment in the MC38 mouse model. The description of tested mouse surrogate molecules is described in the Table 31.

TABLE 31
Description of the mouse surrogate molecules

| Molecule | Molecule characteristics |
|---|---|
| mSOT201 | Mouse anti-PD-1 antibody (clone RMP1-14) mouse IgG1-D265A |
| | RLI-15_{AQA} genetically fused to anti-PD-1 antibody |
| RLI2AQ | RLI-15 having G78A/N79Q stabilizing mutations but no reduced IL- |
| | 2/IL-15Rβγ binding |
| mPD-1 | Mouse anti-PD-1 antibody (clone RMP1-14) mouse IgG1-D265A |

A single dose of mSOT201 of 2 mg/kg (G3) showed about the same therapeutic effect as combined therapies with 4 administrations of 1 mg/kg RLI2_{AQ}+a single dose of 5 mg/kg mPD1 (G8) or with 4 administrations of 1 mg/kg RLI2_{AQ}+a four doses of 5 mg/kg mPD1 (G9). However, a single dose of mSOT201 of 5 mg/kg (G2) outperforms the multiple administrations of the individual components (G8 and G9). (see FIG. 18)

29. Mechanistical Studies on Differences in Immune Cell Activation Under of mSOT201 vs SOT101+Anti-PD-1 Antibody Treatment

The aim of the study was to evaluate the anti-tumor activity of a similar efficacious dose of mSOT201 vs SOT101+anti-PD-1 treatment in the MC38 mouse model. The description of tested mouse surrogate molecules is described in the Table 31.

Differences in the relative number of various immune cell populations upon both treatments were detected in tumor, spleen and lymph nodes. The relative expansion of CD8⁺ T cells and αβTCR bearing CD3⁺ cells did not change between both treatments in spleen and lymph nodes. However, in tumor mSOT201 induced a higher relative increase in CD8⁺ T cells whereas the combined RLI2_{AQ}+anti-PD-1 treatment increased more NK cells. Interestingly, mSOT201 induced a higher percentage of γδTCR bearing CD3⁺ cells in spleen and lymph nodes, while the combined RLI2_{AQ}+anti-PD-1 treatment induced a higher percentage of γδTCR bearing CD3⁺ cells mainly in tumors. (see FIG. 19).

### 30. DC-T Cell-Based Assay and Fluorospot Assay for Determining Immunogenicity

### DC-T Cell Based Assay for Determining Immunogenicity

Buffy coats were obtained from healthy donors. The blood was diluted with PBS-EDTA (to get 175 mL of diluted blood) and PBMCs were isolated by Ficoll Paque gradient (15 mL Ficoll+35 mL diluted blood). CD14⁺ monocytes were isolated using EasySep™ Human CD14 Positive Selection Kit II (17858, StemCell) according to manufacturer's instructions. CD14⁻ fraction was pipetted into a new falcon tube, the rest was centrifuged at 1200 rpm, 10 min, then resuspended in CryoStore media, frozen and temporarily stored at −80° C. Isolated CD14⁺ monocytes were resuspended in DC media (CellGro supplemented with IL-4 and GM-CSF). Cells were incubated at 37° C. with 5% CO₂ for 5 days, harvested and seeded into 48-well plates. iDCs were loaded with proteins for 4 h and maturated with a cytokine cocktail (TNF-α, IL-1β plus IL-4 and GM-CSF) overnight. Washing followed for 4 times with PBS and T cell medium. Cells were co-cultured with autologous, CFSE stained CD4⁺ T cells at a 1:10 ratio (negative magnetic separation) and cultivated for 7 days. CFSE dilution was detected by flow cytometry.

Mix of antibodies and viability dye used for evaluation of CD4⁺ T cell proliferation via flow cytometry (T cells stained with CFSE (FITC)):

| Marker | Fluorochrome | μl/sample | Vendor | Cat. No. | Clone |
|---|---|---|---|---|---|
| CD3 | APC-eFluor 780 | 2 | TFS | 47-0037-42 | OKT3 |
| CD4 | PE-Cy7 | 2 | TFS | 25-0049-42 | RPA-T4 |
| CD8 | PerCP-Cy5.5 | 2 | Biolegend | 344710 | SK1 |
| Zombie Aqua | BV510 | 1 | BioLegend | 423102 | |
| PBS | | 43 | | | |

The aim of the study was to assess the immunogenicity risk of pembrolizumab-based immunocytokines bearing one RLI-15 mutein (PEM-RLI-15 candidate molecules) in vitro. The DC-T cell assay method was used for this purpose, where the test products were first incubated with immature dendritic cells (iDCs) leading to later presentation to autologous T cells as processed peptides of the candidate molecules loaded on the MHC molecules of the matured DCs (mDCs). After a 7-day co-incubation period, T cell proliferation was measured as a surrogate marker for anti-drug antibody formation. The detection of T cell proliferation induced by DCs was used to mitigate the stimulatory activity of the RLI-15 component in the test system that can have a strong influence on the result, which shall not be attributed to immunogenicity. Keyhole limpet hemocyanin (KLH) was used as a positive control, as KLH is known to induce a strong immune response induction. Pembrolizumab was used as a negative control. Control DCs loaded with no protein were used as control for assessment of unspecific T cell proliferation.

TABLE 32
PEM-RLI-15 candidate molecules for DC-T cell-based assay

| Molecule | Molecule characteristics |
|---|---|
| pembrolizumab | humanized anti-PD-1 antibody, Manufacturer MSD |
| (KEYTRUDA ®) | |
| PEM L-RLI DANA x1 | RLI-15 mutations: D158A (D61A), N162A (N65A), G175A, |
| | N176Q |
| | Fc modification: L235E, S228P, T366S, L368A, Y407V, |
| | T366W |
| PEM LY-RLI DANA x1 | RLI-15 mutations: D158A (D61A), N162A (N65A), G175A, |
| | N176Q |
| | Fc modification: L235E, M252Y/S254T/T256E, S228P, |
| | T366S, L368A, Y407V, T366W |
| PEM LY-RLI DANAQD | RLI-15 mutations: D158A (D61A), N162A (N65A), Q198D |
| x1 | (Q101D), G175A, N176Q |
| | Fc modification: L235E, M252Y/S254T/T256E, S228P, |
| | T366S, L368A, Y407V, T366W |

PEM-RLI-15 candidate molecules according to Table 32 were used at two concentrations each for the stimulation of iDCs. Maturation of DCs was induced by proinflammatory cytokines. After 24 h, mDCs were washed and incubated with autologous CD4⁺ T cells that were pre-stained with CFSE. Proliferation of T cells was evaluated based on CFSE detection by flow cytometry after 7 days.

The assay could not be conducted with SOT201 (PEM L-RLI N65A x1), due still too high activity of the RLI N65A mutein leading to the direct T cell activation and spill over the RLI-15 activity.

DCs generated from human CD14⁺ monocytes (11 healthy donors from 3 separate experiments) were incubated with 10 μg/ml (not shown) or 50 μg/ml PEM-RLI-15 candidate molecules, pembrolizumab or KLH for 24 h in the presence of maturation signal (proinflammatory cytokines TNFα and IL-1β). Washed mDCs loaded with proteins were subsequently cultured with autologous, CFSE stained CD4⁺ T cells. T cell proliferation was measured after 7 days by flow cytometry. Proportion of proliferating CD4⁺ T cells was evaluated based on CFSE signal, where CFSE^{low} cells were considered as cycling cells. KLH was used as a positive control, pembrolizumab as a negative control (see FIG. 20A). The PEM-RLI-15 candidate molecule PEM L-RLI DANA x1/PEM LY-RLI DANA x1 did not induce significant proliferation of T cells compared to the negative control reflecting a low immunogenicity risk (positive response detected in 1 out of 11 donors). Candidate molecule PEM LY-RLI DANAQD x1 induced significant proliferation of T cells compared to negative control (p=0.0208, Paired t test), pointing to potential immunogenicity risk (positive response detected in 4 out of 11 donors) for this RLI-15 mutein having 3 mutations for reducing the binding to IL-2/IL-15Rβγ.

### FluoroSpot Assay for Determining Immunogenicity

As a too active RLI-15 mutein is stimulating the immune response, the DC-T cell assay is not suitable to test the immunogenicity of the RLI-15_{AQA} as compared to RLI-15 (wildtype sequence). Accordingly, pairs of peptides having introduced substitutions were generated spanning the substitutions and tested in the Fluorospot assay.

TABLE 33
Tested peptides

| peptide | RLI-15 | Substitutions | sequence |
|---|---|---|---|
| WT2 | RLI-15_{AQ} | wt\|G₁₇₅A\|N₁₇₆Q | ELQVISLESGDASIHDTVENLIILANNSLSSNAQV |
| Mut2 | RLI-15_{AQA} | N₆₅A\|G₁₇₅A\|N₁₇₆Q | ELQVISLESGDASIHDTVEALIILANNSLSSNAQV |
| WT3 | RLI-15 | N₆₅A\|wt\|wt | VEALIILANNSLSSNGNVTESGCKECEELEEK |
| | NA | | |
| Mut3 | RLI-15_{AQA} | N₆₅A\|G₁₇₅A\|N₁₇₆Q | VEALIILANNSLSSNAQVTESGCKECEELEEK |

PBMCs, isolated from each of 40 healthy donors, were retrieved from cryogenic storage and thawed in culture media. CD8 cells were depleted from PBMCs using negative bead selection. CD8-depleted PBMCs were seeded into cell culture plates in RPMI+10% huAB serum and subsequently pulsed with the pooled test peptides, while further cultured in cytokine-supplemented medium. After overnight culture and on day 4 of culture, medium was refreshed containing supporting cytokines IL-7 and IL-2. After 7 days of culture, the enriched CD8-depleted PBMCs were harvested and left to rest overnight. On day 8, the PBMCs were seeded and re-stimulated on the IFN-γ/TNF-α FluoroSpot plates in the presence or absence of the peptide pools and the control molecules. After overnight incubation, T cell activation was assessed by measuring IFN-γ and TNF-α with the Mabtech IRIS FluoroSpot Reader. FIG. 20 B shows that for all test conditions, the confidence intervals overlap with 0 meaning that there is no evidence of a shift in the mean dSFU comparing mutant peptides with the paired wildtype sequence. Therefore, for both the N65A substitution and the G175A/N176Q pair of substitutions, a relevant increase in the immunogenicity is not seen.

### 31. Potency of Different Anti-PD-1 IL-2/IL-5Rβγ Agonist Immunocytokines

The following anti-PD-1 IL-2/IL-15Rβγ agonist immunocytokines (Table 34) were made to compare their activities.

TABLE 34
anti-PD-1 IL-2/IL-15Rβγ agonist immunocytokines

| Molecule | Molecule characteristics |
|---|---|
| SOT201 | heterodimeric anti-PD-1 antibody (derived from IgG4 pembrolizumab with |
| | reduced FcγR binding) |
| | fused C-terminally to one RLI-15_{AQA} |
| | (SEQ ID NO: 22, SEQ ID NO: 38, SEQ ID NO: 24) |
| PD1-IL2v | heterodimeric anti-PD-1 antibody (abolished FcγR binding through LALAPG |
| | substitutions) |
| | fused C-terminally to one IL-2 mutein with T3A, F42A, Y45A, L72G, C125A |
| | substitutions (Klein, Codarri-Deak et al. 2019) |
| | (SEQ ID NO: 51, SEQ ID NO: 52, SEQ ID NO: 53) |
| αhPD1-IL15m | heterodimeric anti-PD-1 antibody (abolished FcγR binding through LALAGA |
| (M2) | substitutions) |
| | fused C-terminally to one IL-15 mutein with N1G, D30N, E46G, V49R, E64Q |
| | (Xu, Carrascosa et al. 2021) |
| | (SEQ ID NO: 60, SEQ ID NO: 61, SEQ ID NO: 62) |
| αhPD1-IL15ml | heterodimeric anti-PD-1 antibody (abolished FcγR binding through LALAGA |
| (M1) | substitutions) |
| | fused C-terminally to one IL-15 mutein with N1A, D30N, E46G, V49R |
| | (SEQ ID NO: 57, SEQ ID NO: 58, SEQ ID NO: 59) |
| Kadmon | heterodimeric anti-PD1 antibody (abolished FcγR binding through LALAPG |
| | substitutions) |
| | N-terminally fused to one RLI-15_{N65S} mutein (Lu, Polonskaya et al. 2020) |
| | (SEQ ID NO: 63, SEQ ID NO: 64, SEQ ID NO: 65) |

The potency of the anti-PD-1 IL-2/IL-15Rβγ agonist immunocytokines was determined on kit225 cells (see Table 35) and hPBMC (see Table 36).

TABLE 35
Potency of anti-PD-1 IL-2/IL-15Rβγ
agonist immunocytokines in kit225 assay

| | EC50 | Relative potency |
|---|---|---|
| | kit225 cells [pM] | to SOT201 |
| SOT201 | 17917 | — |
| PD1-IL2v | 3938 | 455% |
| αhPD1-IL15m2 (M2) | 25372 | 70.62% |
| αhPD1-IL15m1 (M1) | 2682 | 668% |

TABLE 36
Potency of anti-PD-1 IL-2/IL-15Rβγ agonist immunocytokines on hPBMC

| | | | | Relative |
|---|---|---|---|---|
| | EC50 | Relative potency | EC50 | potency |
| | Ki67⁺CD8⁺ | CD8⁺ T cells to | Ki67⁺NK cells | NK cells to |
| | T cells [pM] | SOT201 | [pM] | SOT201 |
| SOT201 | 18828 | — | 5120 | — |
| PD1-IL2v | 4789 | 366 | 858 | 611 |
| αhPD1-IL15m2 (M2) | 6423 | 95 | 7004 | 69 |
| αhPD1-IL15m1 (M1) | 6925 | 430 | 728 | 686 |

### 32. PD-1/PD-L1 Blocking Activity of Anti-PD-1 IL-2/L-5Rβγ Agonist Immunocytokines

To assess the blocking activity of the PD-1/PD-L1 axis, of anti-PD-1 IL-2/IL-15Rβγ agonist immunocytokines were tested using the PD-1/PD-L1 Blockade Bioassay (Promega, No. 11250) as described above. Results are shown in Table 37.

TABLE 37
PD-1/PD-L1 blocking anti-PD-1 IL-2/IL-15Rβγ agonist
immunocytokines in Promega blocking assay

| | EC50 | Relative potency vs |
|---|---|---|
| Sample | [pM] | Keytruda ® |
| Keytruda ® | 1765 | — |
| SOT201 | 2773 | 63.65% |
| PD1-IL2v | 4568 | 38.63% |
| αhPD1-IL15m (M2) | 6580 | 26.82% |

SOT201 shows the highest PD-1/PD-L1 blocking activity of the three tested anti-PD-1 IL-2/IL-15Rβγ agonist immunocytokines.

33. Potency of Human and Mouse Surrogate SOT202 Molecules with Modified Effector Functions on Kit225 Cells

SOT202 is a heterodimeric immunocytokine with an antibody derived from the humanized IgG 1 hCl1a with T366W—knob/T366S, L368A, Y407V-hole substitutions, and deleted terminal K of the heavy chains, fused to RLI-15_{AQA} at the C-terminus of the knob heavy chain (see SEQ ID NO: 50, SEQ ID NO: 49 and SEQ ID NO: 37). In the following examples, the term SOT202-XXX indicates molecules where further mutations of modification have been made to SOT202, such as the DANA mutation in RLI2 as shown in Table 11. For clarity, SOT202-DANA differs from SOT202 only by the additional DA (D61A) mutation, as SOT202 already contains the NA (N65A) mutation (numbers refer to IL-15 numbering). Mutation in the effector domain of the IgG1 molecule modifying ADCC properties of the antibody such as the AAA, DE and DLE mutations as shown in are listed in Table 2.

Table 2. The term “afuc” stands for an afucosylation IgG1 molecule. Afucosylated antibodies have also modified ADCC properties.

The activity of human and murine surrogate SOT202 ADCC-modified molecules on the induction of proliferation of kit225 cells was assessed as described in Example 1, and the EC50 and relative potency compared to SOT101 is shown in Table 38 and Table 39. The murine SOT202 was generated by replacing the human hIgG1 constant domain of SOT202 by its murine equivalent of mIgG2a (mSOT202: SEQ ID NO: 51, SEQ ID NO: 67 and SEQ ID NO: 68; mSOT2020 LALAPG: SEQ ID NO: 69, SEQ ID NO: 70 and SEQ ID NO: 68; mSOT202 isotype: mSOT202 isotype HC knob, SEQ ID NO: 72 and SEQ ID NO: 73; mSOT202 LALAPG isotype: SEQ ID NO: 74, SEQ ID NO: 75. SEQ ID NO: 73).

TABLE 38
Potency of human SOT202 ADCC-modified
molecules on kit225 cells

| | EC50 | Relative potency |
|---|---|---|
| Sample | kit225 cells [pM] | to SOT101 |
| SOT101 | 44.42 | — |
| SOT202 (hCl1a-NA x1) | 14269 | 0.31% |
| SOT202-afuc (hCl1a-afuc-NA | 14316 | 0.31% |
| x1) | | |

| | EC50 | Relative potency to |
|---|---|---|
| Sample | kit225 cells [pM] | PEM-RLI NQD x1 |
| PEM-RLI NQD x1 | 47198 | — |
| SOT202-DANA | 678300 | 6.96% |
| SOT202-afuc-DANA | 463652 | 10.18% |
| SOT202-AAA-DANA | 426399 | 11.07% |
| SOT202-DE-DANA | 534132 | 8.84% |
| SOT202-DLE-DANA | 646221 | 7.30% |

This potency assay shows that SOT202 displays the same potency on kit225 cells as SOT201 (see Table 28) and that ADCC modifications did not affect the potency of the immunocytokines. Therefore, the toolbox allows to tune ADCC activity of the antibodies without affecting the potency of the immunocytokines with respect to activation of kit225 cells.

TABLE 39
Potency of human SOT202 molecules and
mouse SOT202 surrogates on kit225 cells

| | EC50 | Relative potency |
|---|---|---|
| | kit225 cells [pM] | to SOT101 |
| SOT101 | 31.49 | — |
| SOT202 (hCl1a-NA x1) | 9610 | 0.33% |
| mSOT202 | 24011 | 0.13% |
| SOT202 LALAPG | 7828 | 0.40% |
| mSOT202 LALAPG | 17969 | 0.18% |
| SOT202 LALAPG isotype | 13792 | 0.23% |
| mSOT202 LALAPG isotype | 10798 | 0.29% |
| SOT202 isotype | 9560 | 0.33% |
| mSOT202 isotype | 12475 | 0.25% |

As for human SOT202, the ADCC modification (LALAPG mutation) did not affect the potency of the mouse SOT202 surrogates with regards to activation of kit225 cells. However, mouse SOT202 surrogates are less potent than their human counterparts, which likely is due to kit225 cells expressing no CD16 required for co-signaling with IL-15Rγ on human NK cells and mouse NK cells.

### 34. Potency of Human SOT202 ADCC-Modified Molecules on Human NK and CD8⁺ T Cells

The activity of human SOT202 ADCC-modified molecules on the induction of proliferation of human NK and CD8⁺ T cells was assessed as described in Example 1 (hPBMC potency assay), and the EC50 and relative potency compared to S8T202 is shown in FIG. 21 and Table 40.

TABLE 40
Potency of human SOT202 ADCC-modified
molecules on human NK and CD8⁺ T cells

| | EC50 | Relative potency | EC50 | Relative potency |
|---|---|---|---|---|
| | Ki67⁺CD8⁺ | CD8⁺ T cells to | Ki67⁺ NK cells | NK cells to |
| | T cells [pM] | SOT202-DANA | pM | SOT202-DANA |
| SOT202-DANA | 454350.00 | — | 6742.50 | — |
| SOT202-afuc-DANA | 547200.00 | 83% | 783.45 | 861% |
| SOT202-DLE-DANA | 100170.00 | 454% | 117.90 | 5719% |
| SOT202-DE-DANA | 198300.00 | 229% | 186.65 | 3612% |
| SOT202-LALAPG- | 1157000.00 | 39% | 126500.00 | 5% |
| DANA | | | | |

SOT202-DANA with DLE and DE mutation enhancing ADCC greatly increased the human NK cell activity when compared to SOT202-DANA without ADCC-modifications. Afucosylated SOT202 also increased ADCC activity, but to a lesser extent than the DE and DLE mutations. On the other hand, mutations reducing ADCC, such as the LALAPG mutations, almost abolished activation of NK cells. These mutations had only minor effects on CD8⁺ T cells activation. Without being bound by a theory, it is assumed that higher binding to CD16 receptors via enhancing mutations synergizes with the IL-15Rβγ signaling.

### 35. Potency of Human SOT202 Molecules on Human NK and CD8⁺ T Cells Compared to SOT201 Molecules

The activity of human SOT202 molecules on the induction of proliferation of human NK and CD8⁺ T cells was compared to the activity of SOT201. EC50 and relative potency compared to SOT202 and SOT201 is shown in FIG. 22 and Table 41.

TABLE 41
Potency of human SOT202 molecules on human
NK and CD8⁺ T cells, compared to SOT201

| | EC50 | Relative potency | EC50 | Relative potency |
|---|---|---|---|---|
| | Ki67⁺CD8⁺ | CD8⁺ T cells to | Ki67⁺NK | NK cells to |
| | T cells [pM] | SOT201 | cells [pM] | SOT201 |
| SOT201 (PEM L-RLI | 10850.00 | — | 3333.00 | — |
| NAx1) | | | | |
| SOT202 (hCl1a-NA x1) | 22360.00 | 49% | 345.70 | 964% |
| SOT202-afuc (hCl1a- | 12930.00 | 84% | 79.79 | 4177% |
| afuc-NA x1) | | | | |
| SOT201-DANA (PEM L- | 628600.00 | 2% | 190300.00 | 2% |
| RLI DANA x1) | | | | |
| SOT202-DANA | 703300.00 | 2% | 12300.00 | 27% |
| SOT202-afuc-DANA | 382100.00 | 3% | 2232.00 | 149% |

The activity of human SOT202 molecules on the induction of proliferation of human NK and CD8⁺ T cells was compared to the activity of SOT201-DANA. EC50 and relative potency compared to SOT202 and SOT201 is shown in FIG. 23 and Table 41. Decreased stimulatory activity of molecules with DANA mutations, compared to molecules with NA mutations only, confirms the lower stimulatory activity of this mutation as already described in previous examples. SOT202 molecules (SOT202 having the NA mutation) with enhanced ADCC activity via afucosylation increase NK cells activity, but not CD8⁺ T cells activity, and confirms the results shown in Example 34. SOT201 is based on an IgG4 antibody, and as such, an IgG4 antibody, has intrinsic low ADCC activity. Again, without being bound by a theory, it is assumed that higher binding to CD16 receptors of afucosylated molecules synergizes with the IL-15Rβγ signaling.

TABLE 42
Potency of human SOT202 molecules on human
NK and CD8⁺ T cells, compared to SOT201-DANA

| | EC50 | Relative potency | EC50 | Relative potency |
|---|---|---|---|---|
| | Ki67⁺CD8⁺ | CD8⁺ T cells to | Ki67⁺NK cells | NK cells to |
| | T cells [pM] | SOT201-DANA | [pM] | SOT201-DANA |
| SOT201-DANA | 1158000.00 | — | 117700.00 | — |
| (PEM L-RLI DANA | | | | |
| x1) | | | | |
| SOT202-DANA | 975800.00 | 119% | 3697.00 | 3184% |
| SOT202-afuc-DANA | 539000.00 | 215% | 524.40 | 22445% |
| SOT202-LALAPG- | 1237000.00 | 94% | 129000.00 | 91% |
| DANA | | | | |

SOT202 and SOT201 molecules have the same potency on human CD8⁺ T cells, but not on NK cells. Afucosylation increased human NK cell activity.

36. mSOT202 Activates Immune Cells in Spleen of Healthy C57BL/6 Mice

A murine SOT202 was generated by replacing the human hIgG1 constant domain of SOT202 by its murine equivalent of mIgG2a (SEQ ID NO: 66, SEQ ID NO: 67 and SEQ ID NO: 68. Cell proliferation (Ki67) was detected in spleen by flow cytometry 5 days after IV injection of compounds at 5, 10 or 20 mg/kg of mSOT202 in healthy C57BL/6 mice. mSOT202 showed dose-dependent stimulation of NK and CD8⁺ T cells (FIGS. 24 (A) and (B)).

37. mSOT202 Induces Synergy Between ADCC Activity and the RLI2 Stimulation on NK Cell Proliferation

Cell proliferation (Ki67) was detected in spleen by flow cytometry 5 days and 10 days after IV injection of mSOT202 molecules at 5 mg/kg in healthy C57BL/6 mice. The proliferation activity on NK cells of mSOT202 (hCl1a-mIgG2a-NA 1x) was higher than the effect of hCl1a-mIgG2a (molecule without RLI2) added to the effect of mSOT202-LALAPG (hCl1a-mIgG2a-LALAPG-NA 1x, having no ADCC activity) showing a synergy between the ADCC activity of the antibody in mSOT202 and the proliferation activity of RLI2 (FIG. 25(A)) believed to be due to CD16 signalling. ADCC may therefore contribute to the increase activity of NK cells. Synergy could not be measured in this experimental model for CD8⁺ T cell stimulation (FIG. 25 (B)).

### LITERATURE

Abramson, H. N. (2018). “Monoclonal Antibodies for the Treatment of Multiple Myeloma: An Update.” Int J Mol Sci 19(12).
Ahmed, A. A., et al. (2016). “Structural characterization of GASDALIE Fc bound to the activating Fc receptor FcgammaRIIIa.” J Struct Biol 194(1): 78-89.
Alegre, M. L., et al. (1992). “Effect of a single amino acid mutation on the activating and immunosuppressive properties of a “humanized” OKT3 monoclonal antibody.” J Immunol 148(11): 3461-3468.
An, Z., et al. (2009). “IgG2m4, an engineered antibody isotype with reduced Fc function.” MAbs 1(6): 572-579.
Bernett, M. J., et al. (2018). “Abstract 5565: Potency-reduced IL15/IL15Rα heterodimeric Fc-fusions display enhanced in vivo activity through increased exposure.” Cancer Research 78(13 Supplement): 5565-5565.
Bolt, S., et al. (1993). “The generation of a humanized, non-mitogenic CD3 monoclonal antibody which retains in vitro immunosuppressive properties.” Eur J Immunol 23(2): 403-411.
Chester, C., et al. (2018). “Immunotherapy targeting 4-1BB: mechanistic rationale, clinical results, and future strategies.” Blood 131(1): 49-57.
Choi, H. J., et al. (2013). “A heterodimeric Fc-based bispecific antibody simultaneously targeting VEGFR-2 and Met exhibits potent antitumor activity.” Mol Cancer Ther 12(12): 2748-2759.
Choi, H. J., et al. (2015). “Crystal structures of immunoglobulin Fc heterodimers reveal the molecular basis for heterodimer formation.” Mol Immunol 65(2): 377-383.
Chu, S. Y., et al. (2008). “Inhibition of B cell receptor-mediated activation of primary human B cells by coengagement of CD19 and FcgammaRIIb with Fc-engineered antibodies.” Mol Immunol 45(15): 3926-3933.
Clemenceau, B., et al. (2013). “The human natural killer cytotoxic cell line NK-92, once armed with a murine CD16 receptor, represents a convenient cellular tool for the screening of mouse mAbs according to their ADCC potential.” MAbs 5(4): 587-594.
Conlon, K. C., et al. (2019). “Cytokines in the Treatment of Cancer.” J Interferon Cytokine Res 39(1): 6-21.
Dall'Acqua, W. F., et al. (2002). “Increasing the affinity of a human IgG1 for the neonatal Fc receptor: biological consequences.” J Immunol 169(9): 5171-5180.
Darvin, P., et al. (2018). “Immune checkpoint inhibitors: recent progress and potential biomarkers.” Exp Mol Med 50(12): 165.
Davis, J. H., et al. (2010). “SEEDbodies: fusion proteins based on strand-exchange engineered domain (SEED) CH3 heterodimers in an Fc analogue platform for asymmetric binders or immunofusions and bispecific antibodies.” Protein Eng Des Sel 23(4): 195-202.
De Sousa Linhares, A., et al. (2018). “Not All Immune Checkpoints Are Created Equal.” Frontiers in Immunology 9(1909).
Diebolder, C. A., et al. (2014). “Complement is activated by IgG hexamers assembled at the cell surface.” Science 343(6176): 1260-1263.
Dolgin, E. (2020). “Antibody engineers seek optimal drug targeting TIGIT checkpoint.” Nat Biotechnol 38(9): 1007-1009.
Edgar, R. C. (2004). “MUSCLE: multiple sequence alignment with high accuracy and high throughput.” Nucleic Acids Res 32(5): 1792-1797.
Elliott, J. M., et al. (2014). “Antiparallel conformation of knob and hole aglycosylated half-antibody homodimers is mediated by a CH2-CH3 hydrophobic interaction.” J Mol Biol 426(9): 1947-1957.
Forero-Torres, A., et al. (2012). “Results of a phase 1 study of AME-133v (LY2469298), an Fc-engineered humanized monoclonal anti-CD20 antibody, in FcgammaRIIIa-genotyped patients with previously treated follicular lymphoma.” Clin Cancer Res 18(5): 1395-1403.
Fu, Y., et al. (2020). “Therapeutic strategies for the costimulatory molecule OX40 in T-cell-mediated immunity.” Acta Pharmaceutica Sinica B 10(3): 414-433.
Giron-Michel, J., et al. (2005). “Membrane-bound and soluble IL-15/IL-15Ralpha complexes display differential signaling and functions on human hematopoietic progenitors.” Blood 106(7): 2302-2310.
Godar, M., et al. (2018). “Therapeutic bispecific antibody formats: a patent applications review (1994-2017).” Expert Opinion on Therapeutic Patents 28(3): 251-276.
Goujon, M., et al. (2010). “A new bioinformatics analysis tools framework at EMBL-EBI.” Nucleic Acids Res 38(Web Server issue): W695-699.
Gunasekaran, K., et al. (2010). “Enhancing antibody Fc heterodimer formation through electrostatic steering effects: applications to bispecific molecules and monovalent IgG.” J Biol Chem 285(25): 19637-19646.
Hangasky, J. A., et al. (2020). “Interleukin 15 Pharmacokinetics and Consumption by a Dynamic Cytokine Sink.” Frontiers in Immunology 11(1813).
Hon, T., et al. (1987). “Establishment of an interleukin 2-dependent human T cell line from a patient with T cell chronic lymphocytic leukemia who is not infected with human T cell leukemia/lymphoma virus.” Blood 70(4): 1069-1072.
Idusogie, E. E., et al. (2001). “Engineered antibodies with increased activity to recruit complement.” J Immunol 166(4): 2571-2575.
Lazar, G. A., et al. (2006). “Engineered antibody Fc variants with enhanced effector function.” Proc Natl Acad Sci USA 103(11): 4005-4010.
Leabman, M. K., et al. (2013). “Effects of altered FcgammaR binding on antibody pharmacokinetics in cynomolgus monkeys.” MAbs 5(6): 896-903.
Leaver-Fay, A., et al. (2016). “Computationally Designed Bispecific Antibodies using Negative State Repertoires.” Structure 24(4): 641-651.
Lo, M., et al. (2017). “Effector-attenuating Substitutions That Maintain Antibody Stability and Reduce Toxicity in Mice.” J Biol Chem 292(9): 3900-3908.
Lu, X., et al. (2019). “Deamidation and isomerization liability analysis of 131 clinical-stage antibodies.” MAbs 11(1): 45-57.
Merchant, A. M., et al. (1998). “An efficient route to human bispecific IgG.” Nat Biotechnol 16(7): 677-681.
Mimoto, F., et al. (2013). “Novel asymmetrically engineered antibody Fc variant with superior FcgammaR binding affinity and specificity compared with afucosylated Fc variant.” MAbs 5(2): 229-236.
Moore, G. L., et al. (2011). “A novel bispecific antibody format enables simultaneous bivalent and monovalent co-engagement of distinct target antigens.” MAbs 3(6): 546-557.
Moore, G. L., et al. (2010). “Engineered Fc variant antibodies with enhanced ability to recruit complement and mediate effector functions.” MAbs 2(2): 181-189.
Natsume, A., et al. (2008). “Engineered antibodies of IgG1/IgG3 mixed isotype with enhanced cytotoxic activities.” Cancer Res 68(10): 3863-3872.
Needleman, S. B. and C. D. Wunsch (1970). “A general method applicable to the search for similarities in the amino acid sequence of two proteins.” J Mol Biol 48(3): 443-453.
Nellis, D. F., et al. (2012). “Characterization of recombinant human IL-15 deamidation and its practical elimination through substitution of asparagine 77.” Pharm Res 29(3): 722-738.
Nordstrom, J. L., et al. (2011). “Anti-tumor activity and toxicokinetics analysis of MGAH22, an anti-HER2 monoclonal antibody with enhanced Fcgamma receptor binding properties.” Breast Cancer Res 13(6): R123.
Pearson, W. R. and D. J. Lipman (1988). “Improved tools for biological sequence comparison.” Proc Natl Acad Sci USA 85(8): 2444-2448.
Pini, A., et al. (1998). “Design and use of a phage display library. Human antibodies with subnanomolar affinity against a marker of angiogenesis eluted from a two-dimensional gel.” J Biol Chem 273(34): 21769-21776.
Richards, J. O., et al. (2008). “Optimization of antibody binding to FcgammaRIIa enhances macrophage phagocytosis of tumor cells.” Mol Cancer Ther 7(8): 2517-2527.
Ring, A. M., et al. (2012). “Mechanistic and structural insight into the functional dichotomy between IL-2 and IL-15.” Nat Immunol 13(12): 1187-1195.
Robinson, N. E. and A. B. Robinson (2004). “Prediction of primary structure deamidation rates of asparaginyl and glutaminyl peptides through steric and catalytic effects.” J Pept Res 63(5): 437-448.
Robinson, T. O. and K. S. Schluns (2017). “The potential and promise of IL-15 in immuno-oncogenic therapies.” Immunol Lett 190: 159-168.
Rother, R. P., et al. (2007). “Discovery and development of the complement inhibitor eculizumab for the treatment of paroxysmal nocturnal hemoglobinuria.” Nat Biotechnol 25(11): 1256-1264.
Shang, L., et al. (2014). “Selective antibody intervention of Toll-like receptor 4 activation through Fc gamma receptor tethering.” J Biol Chem 289(22): 15309-15318.
Shields, R. L., et al. (2001). “High resolution mapping of the binding site on human IgG1 for Fc gamma RI, Fc gamma RII, Fc gamma RIII, and FcRn and design of IgG1 variants with improved binding to the Fc gamma R.” J Biol Chem 276(9): 6591-6604.
Smith, T. F. and M. S. Waterman (1981). “Comparison of biosequences.” Advances in Applied Mathematics 2(4): 482-489.
Sola, R. J. and K. Griebenow (2009). “Effects of glycosylation on the stability of protein pharmaceuticals.” J Pharm Sci 98(4): 1223-1245.
Solomon, B. L. and I. Garrido-Laguna (2018). “TIGIT: a novel immunotherapy target moving from bench to bedside.” Cancer Immunol Immunother 67(11): 1659-1667.
Soman, G., et al. (2009). “MTS dye based colorimetric CTLL-2 cell proliferation assay for product release and stability monitoring of interleukin-15: assay qualification, standardization and statistical analysis.” J Immunol Methods 348(1-2): 83-94.
Spiess, C., et al. (2015). “Alternative molecular formats and therapeutic applications for bispecific antibodies.” Mol Immunol 67(2 Pt A): 95-106.
Stavenhagen, J. B., et al. (2007). “Fc optimization of therapeutic antibodies enhances their ability to kill tumor cells in vitro and controls tumor expansion in vivo via low-affinity activating Fcgamma receptors.” Cancer Res 67(18): 8882-8890.
Tao, M. H. and S. L. Morrison (1989). “Studies of aglycosylated chimeric mouse-human IgG. Role of carbohydrate in the structure and effector functions mediated by the human IgG constant region.” J Immunol 143(8): 2595-2601.
Thaysen-Andersen, M., et al. (2016). “Recombinant human heterodimeric IL-15 complex displays extensive and reproducible N- and O-linked glycosylation.” Glycoconj J 33(3): 417-433.
Toutain, P. L. and A. Bousquet-Melou (2004). “Plasma terminal half-life.” J Vet Pharmacol Ther 27(6): 427-439.
Vafa, O., et al. (2014). “An engineered Fc variant of an IgG eliminates all immune effector functions via structural perturbations.” Methods 65(1): 114-126.
Villa, A., et al. (2008). “A high-affinity human monoclonal antibody specific to the alternatively spliced EDA domain of fibronectin efficiently targets tumor neo-vasculature in vivo.” Int J Cancer 122(11): 2405-2413.
Von Kreudenstein, T. S., et al. (2013). “Improving biophysical properties of a bispecific antibody scaffold to aid developability: quality by molecular design.” MAbs 5(5): 646-654.
Vonderheide, R. H. (2020). “CD40 Agonist Antibodies in Cancer Immunotherapy.” Annual Review of Medicine 71(1): 47-58.
Waldmann, T. A. (2015). “The shared and contrasting roles of IL2 and IL15 in the life and death of normal and neoplastic lymphocytes: implications for cancer therapy.” Cancer Immunol Res 3(3): 219-227.
Waldmann, T. A., et al. (2020). “IL-15 in the Combination Immunotherapy of Cancer.” Frontiers in Immunology 11(868).
Waldmeier, L., et al. (2016). “Transpo-mAb display: Transposition-mediated B cell display and functional screening of full-length IgG antibody libraries.” MAbs 8(4): 726-740.
Walker, M. R., et al. (1989). “Aglycosylation of human IgG1 and IgG3 monoclonal antibodies can eliminate recognition by human cells expressing Fc gamma RI and/or Fc gamma RII receptors.” Biochem J 259(2): 347-353.
Wang, X., et al. (2018). “IgG Fc engineering to modulate antibody effector functions.” Protein Cell 9(1): 63-73.
Wei, X., et al. (2001). “The Sushi domain of soluble IL-15 receptor alpha is essential for binding IL-15 and inhibiting inflammatory and allogenic responses in vitro and in vivo.” J Immunol 167(1): 277-282.
Xu, D., et al. (2000). “In vitro characterization of five humanized OKT3 effector function variant antibodies.” Cell Immunol 200(1): 16-26.
Yu, X., et al. (2017). “Beyond Antibodies as Binding Partners: The Role of Antibody Mimetics in Bioanalysis.” Annu Rev Anal Chem (Palo Alto Calif) 10(1): 293-320.
Zalevsky, J., et al. (2010). “Enhanced antibody half-life improves in vivo activity.” Nat Biotechnol 28(2): 157-159.
Zhang, T., et al. (2018). “The binding of an anti-PD-1 antibody to FcγRI has a profound impact on its biological functions.” Cancer Immunology. Immunotherapy 67(7): 1079-1090.

1. An interleukin-15 (IL-15) variant comprising amino acid substitutions at position G78 and at position N79 of a mature human IL-15.

2. The IL-15 variant of claim 1, wherein the IL-15 variant comprises the amino acid substitutions G78A, G78V, G78L or G78I, and N79Q, N79H or N79M.

3. The IL-15 variant of claim 1, wherein the IL-15 variant has been expressed in a mammalian cell line.

4. The IL-15 variant of claim 1, wherein the amino acid substitutions
(a) reduce deamidation at N77 and glycosylation at N79 of the IL-15 variant compared to mature human IL-15,
(b) result in less than 30% of glycosylated IL-15 variant, and/or,
(c) increase glycosylation at N71 of the IL-15 variant compared to mature human IL-15.

5. The IL-15 variant of claim 1, wherein the amino acid substitutions do not substantially reduce the IL-15 activity of the IL-15 variant on the proliferation induction of Kit225 cells, 32Db cells, human PBMC or in the Promega IL-15-bioassay.

6. The IL-15 variant of claim 1, wherein the IL-15 variant does not have a substitution at position N71 and/or at position N77.

7. The IL-15 variant of claim 1, wherein the IL-15 variant comprises at least one further substitution that reduces the binding to the IL-2/IL-15Rβ and/or to the γc receptor,
wherein
(a) the site for the further substitution reducing binding to the IL-2/IL-15Rβ and/or to the γc receptor is selected from the list consisting of N1, N4, S7, D8, K10, K11, D30, D61, E64, N65, L69, N72, E92, Q101, Q108, and I111;
(b) the further substitution reducing binding to the IL-2/IL-15Rβ and/or to the γc receptor is selected the list consisting of N1D, N1A, N1G, N4D, S7Y, S7A, D8A, D8N, K10A, K11A, D30N, D61A, D61N, E64Q, N65D, N65A, N65E, N65R, N65K, L69R, N72R, Q101D, Q101E, Q108D, Q108A, Q108E and Q108R; or
(c) the further substitution reducing binding to the IL-2/IL-15Rβ and/or to the γc receptor is a combined substitution and is selected form the list consisting of D8N/N65A, D61A/N65A and D61A/N65A/Q101D.

8. A conjugate comprising an IL-15 variant of claim 1, wherein the conjugate further comprises the sushi domain of an IL-15Rα or a derivative thereof.

9. A fusion protein comprising an IL-15 variant of claim 1, wherein in the fusion protein further comprises the sushi domain of an IL-15Rα or a derivative thereof, a targeting moiety, and/or a half-life extending moiety.

10. The fusion protein of claim 9, wherein the fusion protein comprises, the human IL-15Rα sushi domain, a linker and the IL-15 variant of claim 1, wherein the linker has a length of 18 to 22 amino acids and is composed of serines and glycines.

11. The fusion protein of claim 9, wherein the targeting moiety is an antibody or functional variant thereof.

12. A nucleic acid encoding the IL-15 variant of claim 1.

13. A vector comprising the nucleic acid of claim 12.

14. A host cell comprising the nucleic acid of claim 12 or a vector comprising the nucleic acid.

15. A method of treatment of a subject suffering from, at risk of developing and/or being diagnosed for a neoplastic disease or an infectious disease, the method comprising administering the IL-15 variant of claim 1 to said subject.

16. A pharmaceutical composition comprising the IL-15 variant of claim 1 and a pharmaceutically acceptable carrier.

17. A method of treatment of a subject suffering from, at risk of developing and/or being diagnosed for a neoplastic disease or an infectious disease, the method comprising administering the conjugate of claim 8 to said subject.

18. The IL-15 variant of claim 1, wherein the IL-15 variant comprises at least one further substitution that reduces the binding to the IL-15Rα,
wherein
(a) the site for the further substitution reducing binding to the IL-15Rα is selected from the list consisting of L44, L45, E46, L47, V49, I50, S51, E64, L66, I67, I68 and L69,
(b) the further substitution reducing binding to the IL-15Rα is selected from the list consisting of L44D, E46K, E46G, L47D, V49D, V49R, I50D, L66D, L66E, I67D, and I67E, or
(c) the further substitution reducing binding to the IL-15Rα is a combined substitution selected form the list consisting of E46G/V49R, NIA/D30N/E46G/V49R, N1G/D30N/E46G/V49R/E64Q, V49R/E46G/NIA/D30N and V49R/E46G/N1G/E64Q/D30N.

19. A method of treatment of a subject suffering from, at risk of developing and/or being diagnosed for a neoplastic disease or an infectious disease, the method comprising administering the fusion protein of claim 9 to said subject.

20. A method of treatment of a subject suffering from, at risk of developing and/or being diagnosed for a neoplastic disease or an infectious disease, the method comprising administering the nucleic acid of claim 12 to said subject.

21. A method of treatment of a subject suffering from, at risk of developing and/or being diagnosed for a neoplastic disease or an infectious disease, the method comprising administering the vector of claim 13 to said subject.
